SEQUENCE LISTING (39 sequences)

SEQ ID NO: 1 - DNA, 23 bp, synthetic construct
  source 1..23: mol_type = other DNA; note = EC.stx1-F3 primer to detect E. coli O157:H7
tgatttttcacatgttacctttc

SEQ ID NO: 2 - DNA, 18 bp, synthetic construct
  source 1..18: mol_type = other DNA; note = EC.stx1-B3 primer to detect E. coli O157:H7
taacatcgctcttgccac

SEQ ID NO: 3 - DNA, 38 bp, synthetic construct
  source 1..38: mol_type = other DNA; note = EC.stx1-FIP primer to detect E. coli O157:H7
cctgcaacacgctgtaacgtcaggtacaacagcggtta

SEQ ID NO: 4 - DNA, 38 bp, synthetic construct
  source 1..38: mol_type = other DNA; note = EC.stx1-BIP primer to detect E. coli O157:H7
agtcgtacggggatgcagatagtgaggttccactatgc

SEQ ID NO: 5 - DNA, 25 bp, synthetic construct
  source 1..25: mol_type = other DNA; note = EC.stx1-LF primer to detect E. coli O157:H7
gtatagctactgtcaccagacaatg

SEQ ID NO: 6 - DNA, 22 bp, synthetic construct
  source 1..22: mol_type = other DNA; note = EC.stx1-LB primer to detect E. coli O157:H7
aaatcgccattcgttgactact

SEQ ID NO: 7 - DNA, 20 bp, synthetic construct
  source 1..20: mol_type = other DNA; note = Universal.Bacteroidales.16S rRNA.1.F3 primer
tgcgggtatcgaacaggatt

SEQ ID NO: 8 - DNA, 20 bp, synthetic construct
  source 1..20: mol_type = other DNA; note = Universal.Bacteroidales.16S rRNA.1.B3 primer
ggtaaggttcctcgcgtatc

SEQ ID NO: 9 - DNA, 41 bp, synthetic construct
  source 1..41: mol_type = other DNA; note = Universal.Bacteroidales.16S rRNA.1.FIP primer
ttaacgctttcgcttggccacagtagtccgcacggtaaacg

SEQ ID NO: 10 - DNA, 39 bp, synthetic construct
  source 1..39: mol_type = other DNA; note = Universal.Bacteroidales.16S rRNA.1.BIP primer
gtacgccggcaacggtgaaaacatgttcctccgcttgtg

SEQ ID NO: 11 - DNA, 17 bp, synthetic construct
  source 1..17: mol_type = other DNA; note = Universal.Bacteroidales.16S rRNA.1.LF primer
ggccgaacagcgagcat

SEQ ID NO: 12 - DNA, 19 bp, synthetic construct
  source 1..19: mol_type = other DNA; note = Universal.Bacteroidales.16S rRNA.1.LB primer
caaaggaattgacgggggc

SEQ ID NO: 13 - DNA, 19 bp, synthetic construct
  source 1..19: mol_type = other DNA; note = GenBac3.FP primer
ggggttctgagaggaaggt

SEQ ID NO: 14 - DNA, 20 bp, synthetic construct
  source 1..20: mol_type = other DNA; note = GenBac3.RP primer
ccgtcatccttcacgctact

SEQ ID NO: 15 - DNA, 27 bp, synthetic construct
  source 1..27: mol_type = other DNA; note = GenBac3.Probe
  misc_feature 1: note = fluorescent dye label on 5' end
  misc_feature 27: note = /IAbkFQ/ quencher located at 3' end
  misc_feature 9..10: note = /ZEN/ internal quencher
caatattcctcactgctgcctcccgta

SEQ ID NO: 16 - DNA, 19 bp, synthetic construct
  source 1..19: mol_type = other DNA; note = Universal.Bacteroidales.16S rRNA.2.F3 primer
gatggggatgcgttccatt

SEQ ID NO: 17 - DNA, 20 bp, synthetic construct
  source 1..20: mol_type = other DNA; note = Universal.Bacteroidales.16S rRNA.2.B3 primer
ccatagggcagtcatccttc

SEQ ID NO: 18 - DNA, 39 bp, synthetic construct
  source 1..39: mol_type = other DNA; note = Universal.Bacteroidales.16S rRNA.2.FIP primer
ccaatgtgggggaccttcctcggtaacggcccacctagt

SEQ ID NO: 19 - DNA, 40 bp, synthetic construct
  source 1..40: mol_type = other DNA; note = Universal.Bacteroidales.16S rRNA.2.BIP primer
cacggtccaaactcctacggggctacttggctggttcagg

SEQ ID NO: 20 - DNA, 19 bp, synthetic construct
  source 1..19: mol_type = other DNA; note = Universal.Bacteroidales.16S rRNA.2.LF primer
tgctacctatccccaagac

SEQ ID NO: 21 - DNA, 21 bp, synthetic construct
  source 1..21: mol_type = other DNA; note = Universal.Bacteroidales.16S rRNA.2.LB primer
aggaatattggtcaatgggcg

SEQ ID NO: 22 - DNA, 20 bp, synthetic construct
  source 1..20: mol_type = other DNA; note = Universal.Bacteroidales.16S rRNA.3.F3 primer
aggatgaaggtcctacggat

SEQ ID NO: 23 - DNA, 19 bp, synthetic construct
  source 1..19: mol_type = other DNA; note = Universal.Bacteroidales.16S rRNA.3.B3 primer
agccccaaactttcacagc

SEQ ID NO: 24 - DNA, 39 bp, synthetic construct
  source 1..39: mol_type = other DNA; note = Universal.Bacteroidales.16S rRNA.3.FIP primer
ggcacggagttagccgatgcgggaataaaccctcccacg

SEQ ID NO: 25 - DNA, 38 bp, synthetic construct
  source 1..38: mol_type = other DNA; note = Universal.Bacteroidales.16S rRNA.3.BIP primer
gcagccgcggtaatacggagcttaacgacccgtctgcg

SEQ ID NO: 26 - DNA, 25 bp, synthetic construct
  source 1..25: mol_type = other DNA; note = Universal.Bacteroidales.16S rRNA.3.LF primer
tcataaggtacatacaagctcccac

SEQ ID NO: 27 - DNA, 24 bp, synthetic construct
  source 1..24: mol_type = other DNA; note = Universal.Bacteroidales.16S rRNA.3.LB primer
ccggatttattgggtttaaaggga

SEQ ID NO: 28 - DNA, 21 bp, synthetic construct
  source 1..21: mol_type = other DNA; note = cattle specific Bacteroidales CowM2.FP primer
cggccaaatactcctgatcgt

SEQ ID NO: 29 - DNA, 24 bp, synthetic construct
  source 1..24: mol_type = other DNA; note = cattle specific Bacteroidales CowM2.RP primer
gcttgttgcgttccttgagataat

SEQ ID NO: 30 - DNA, 36 bp, synthetic construct
  source 1..36: mol_type = other DNA; note = cattle specific Bacteroidales CowM2.probe
  misc_feature 1: note = /FAM/ fluorescent dye label at 5' end
  misc_feature 9..10: note = /ZEN/ internal quencher
  misc_feature 36: note = /IABkFQ/ quencher at 3' end
aggcacctatgtcctttacctcatcaactacagaca

SEQ ID NO: 31 - DNA, 27 bp, synthetic construct
  source 1..27: mol_type = other DNA; note = pig specific Bacteroidales Pig-2-Bac41F primer
gcatgaatttagcttgctaaatttgat

SEQ ID NO: 32 - DNA, 22 bp, synthetic construct
  source 1..22: mol_type = other DNA; note = pig specific Bacteroidales Pig-2-Bac163Rm primer
acctcatacggtattaatccgc

SEQ ID NO: 33 - DNA, 14 bp, synthetic construct
  source 1..14: mol_type = other DNA; note = pig specific Bacteroidales Pig-2Bac113MGB
  misc_feature 1: note = /FAM/ fluorescent dye label at 5' end
  misc_feature 9..10: note = /ZEN/ internal quencher
  misc_feature 14: note = /IABkFQ/ quencher at 3' end
tccacgggatagcc

SEQ ID NO: 34 - DNA, 20 bp, synthetic construct
  source 1..20: mol_type = other DNA; note = human specific Bacteroidales HF183F primer
atcatgagttcacatgtccg

SEQ ID NO: 35 - DNA, 20 bp, synthetic construct
  source 1..20: mol_type = other DNA; note = human specific Bacteroidales BFDRev primer
cgtaggagtttggaccgtgt

SEQ ID NO: 36 - DNA, 26 bp, synthetic construct
  source 1..26: mol_type = other DNA; note = human specific Bacteroidales BFDFAM
  misc_feature 1: note = /FAM/ fluorescent dye label at 5' end
  misc_feature 9..10: note = /ZEN/ internal quencher
  misc_feature 26: note = /IABkFQ/ quencher at 3' end
ctgagaggaaggtcccccacattgga

SEQ ID NO: 37 - DNA, 22 bp, synthetic construct
  source 1..22: mol_type = other DNA; note = poultry specific Bacteroidales qCD362_F primer
aatattggtcaatgggcgagag

SEQ ID NO: 38 - DNA, 24 bp, synthetic construct
  source 1..24: mol_type = other DNA; note = poultry specific Bacteroidales qCD364_R primer
cacgtagtgtccgttattccctta

SEQ ID NO: 39 - DNA, 17 bp, synthetic construct
  source 1..17: mol_type = other DNA; note = poultry specific Bacteroidales qCD294_P probe
  misc_feature 1: note = /FAM/ fluorescent dye label at 5' end
  misc_feature 9..10: note = /ZEN/ internal quencher
  misc_feature 17: note = /IABkFQ/ quencher at 3' end
tccttcacgctacttgg

(19) United States
(12) Patent Application Publication
(10) Pub. No.: US 2024/0401103 A1
(43) Pub. Date: Dec. 5, 2024

(54) METHODS FOR MICROBIAL SOURCE TRACKING USING MICROFLUIDIC ANALTYICAL DEVICES

(71) Applicant: Purdue Research Foundation, West Lafayette, IN (US)

(72) Inventors: Mohit VERMA, West Lafayette, IN (US); Jiangshan WANG, West Lafayette, IN (US); Bilal AHMED, West Lafayette, IN (US); Ashley Deniz KAYABASI, Goshen, KY (US)

(21) Appl. No.: 18/674,833

(22) Filed: May 25, 2024

Related U.S. Application Data
(60) Provisional application No. 63/542,271, filed on Oct. 3, 2023.
(60) Provisional application No. 63/469,101, filed on May 26, 2023.
(60) Provisional application No. 63/469,078, filed on May 26, 2023.

(51) Int. Cl. C12Q 1/06; C12Q 1/6844

(52) U.S. Cl. CPC C12Q 1/06; C12Q 1/6844; G01N 33/245; C12Q 2600/16

(57) ABSTRACT

Methods for site-specific risk evaluation of foodborne pathogens and/or fecal indicator bacteria contamination. Methods for generating a heat map of a field showing contamination concentrations integrated with environmental data are also provided.

### PRIORITY

This application is related to and claims the priority benefit of: (a) U.S. Provisional Patent Application No. 63/469,078 filed May 26, 2023; (b) U.S. Provisional Patent Application 63/469,101 filed May 26, 2023; and (c) U.S. Provisional Patent Application No. 63/542,271 filed Oct. 3, 2023. The contents of the aforementioned applications are hereby incorporated in their entireties into this disclosure.

### STATEMENT OF GOVERNMENT SUPPORT

This invention was made with government support under: 1) Grant No. USDA-AMS-TM-SCBGP-G-20-0003 awarded by the United States Department of Agriculture; 2) CPS Award No. 2021CPS12 awarded by the Center for Produce Safety; and 3) CDFA Agreement No. 20-0001-054-SF awarded by the California Department of Food and Agriculture. The United States Government has certain rights in the invention.

### TECHNICAL FIELD

The present disclosure relates to methods of assessing risk related to contamination of fresh produce fields and methods for microbial source tracking.

### SEQUENCE LISTINGS

The sequences herein are also provided in computer readable form encoded in a file filed herewith and incorporated herein by reference, which was created on May 25, 2024, named 70268-02_SequenceListing_25MAY2024.xml, and is 49,532 bytes in size. The information recorded in computer readable form is identical to the written Sequence Listings provided below, pursuant to 37 C.F.R. § 1.821 (f).

### BACKGROUND

This section introduces aspects that may help facilitate a better understanding of the disclosure. Accordingly, these statements are to be read in this light and are not to be understood as admissions about what is or is not prior art.

Foodborne pathogens and fecal contamination on fresh produce can lead to serious health issues and are a public health concern due to the risk of foodborne illnesses. The source of these pathogens can be wild animals or animal feeding operations, for example. Current methods for assessing the risk of contamination on fresh produce are costly, time-consuming, and laboratory based.

The majority of foodborne pathogens linked to fresh produce (i.e., diarrheagenic Escherichia coli, Salmonella enterica, and Listeria monocytogenes) are enteric in origin and fecal contamination can occur anywhere along the farm-to-fork chain. Alegbeleye et al., Sources and contamination routes of microbial pathogens to fresh produce during field cultivation: a review, Food Microbiology 73:177-208 (2018); Bozkurt et al., Assessment of microbial risk during Australian industrial practices for Escherichia coli O157:H7 in fresh cut-cos lettuce: a stochastic quantitative approach, Food Microbiology 95:103691 (2021); Hoelzer et al., Emerging needs and opportunities in foodborne disease detection and prevention: from tools to people, Food Microbiology 75:65-71 (2018); Qi et al., Glove-mediated transfer of Listeria monocytogenes on fresh-cut cantaloupe, Food Microbiology 88:103396 (2020). The use of poorly composted animal manures, substandard irrigation waters, wild animal encroachment, and the spread of airborne bacteria (bioaerosols) from nearby livestock operations are all potential points of entry while growing fresh produce. Alegbeleye et al. (2018), supra; Chen et al., Prevalence and methodologies for detection, characterization and subtyping of Listeria monocytogenes and L. ivanovii in foods and environmental sources, Food Science & Human Wellness 6:97-120 (2017); Li et al., Filtration assisted pretreatment for rapid enrichment and accurate detection of Salmonella in vegetables, Food Science & Human Wellness 12:1167-1173 (2023).

As the consumption of fresh produce increases, public health officials and organizations have pushed for improvements in food safety procedures and environmental assessments to reduce the risk of contamination. Visual inspects and the establishment of “buffer zones” between animal feeding operations and producing fields are the current best practices for environmental assessments. However, a generalized distance guideline and visual inspections may not be enough to account for all environmental risk variables.

Many fresh produce organizations have devised and implemented safety practices and protocols to reduce potential sources of contamination. The California Leafy Greens Marketing Agreement (LGMA) Food Safety Standards, for example, specify that the best practice for environmental assessments is to inspect the production field and surrounding area for potential animal hazards or other sources of human pathogens of concern. A “buffer zone” of 400 feet for animal feeding operations (less than 1,000 animals) or 1200 feet for concentrated animal feeding operations (1,000-80,000 animals) around the production field is required to prevent pathogen transmission from animals to crops (LGMA, 2021). However, the fact that each farm has a unique combination of environmental risk variables (e.g., topography, land-use interactions, and weather) makes this generalized distance guideline difficult to justify. Strawn et al., Landscape and Meteorological Factors Affecting Prevalence of Three Food-Borne Pathogens in Fruit and Vegetable Farms, Applied Environmental Microbiology 79:588-600 (2013a); Strawn et al., Risk Factors Associated with Salmonella and Listeria monocytogenes Contamination of Produce Fields, Applied Environmental Microbiology 79:7618-7627 (2013b). Furthermore, while these practices were initially used to limit food safety risks for preharvest production, applying them to all fields without specificity would elevate production costs (for low-risk fields) and raise produce safety concerns (for high-risk fields). Indeed, LGMA acknowledges that there is limited information on which to base this recommendation, and ideally an appropriate “buffer zone” should be customized to each farm. Hoar, Developing buffer zone distances between sheep grazing operations and vegetable crops to maximize food safety, Center for Produce Safety (2011); Strawn et al. (2013b), supra.

In the majority of cases, the concentration of the enteric pathogens is relatively low, which makes it difficult to identify and enumerate their presence. Lemarchand & Lebaron, Occurrence of Salmonella spp. and Cryptosporidium spp. in a French coastal watershed: relationship with fecal indicators, FEMS Microbiology Letters 218 (1): 203-209 (2003); Ferone et al., Microbial detection and identification methods: bench top assays to omics approaches, Comprehensive Review Food Science & Food Safety 19 (6): 3106-3129 (2020). Additionally, due to the high level of heterogeneity in fresh produce products, pretreatments are typically required where the pretreatment process also dilutes the pathogens. U.S. Food & Drug Administration (FDA), Bacteriological Analytical Manual (BAM), FDA (2021); Ferone et al. (2020), supra. Enteric pathogens can enter a viable but non-culturable state (VBNC) and maintain a low level of metabolic activity without growing on typical microbial media, therefore escaping detection using culture-based approaches Martínez-Vaz et al., Enteric pathogen-plant interactions: molecular connections leading to colonization and growth and implications for food safety, Microbiology Environments 29 (2): 123-135 (2014); Oliver, The viable but nonculturable state in bacteria, J Microbiology (Seoul, Korea) 43 Spec No 93-100 (2005).

Nevertheless, the presence of pathogenic enteric microorganisms on fresh produce poses a significant health risk to humans. Since it is difficult to quantify the abundance of all potential pathogens, it is common practice to only quantify the presence/absence of one or a few fecal indicator bacteria (FIB) (such as Escherichia coli, Enterococcus faecalis, and Bacteroidales) which are microorganisms that have been selected as indicators of fecal contamination. Brauwere & Servais, Modeling fecal indicator bacteria concentrations in natural surface waters: a review, Critical Reviews in Environmental Science & Technology 44 (21): 2380-2453 (2014); Denis et al., Prevalence and trends of bacterial contamination in fresh fruits and vegetables sold at retail in Canada, Food Control 67:225-234 (2016); Drozd et al., Evaluating the Occurrence of Host-Specific Bacteroidales, General Fecal Indicators, and Bacterial Pathogens in a Mixed-Use Watershed, J Environmental Quality 42:713-725 (2013); Harris et al., Fecal Contamination on Produce from Wholesale and Retail Food Markets in Dhaka, Bangladesh, Am J Tropical Medicine & Hygiene 98:287-294 (2017); Ordaz et al., Persistence of Bacteroidales and other fecal indicator bacteria on inanimated materials, melon and tomato at various storage conditions, Int'l J Food Microbiology 299:33-38 (2019).

FIB, such as Escherichia coli, Enterococcus faecalis, and Bacteroidales, are commonly used to assess microbial water quality, but have not conventionally been used outside of this scope. Allende et al., Quantitative microbial exposure modelling as a tool to evaluate the impact of contamination level of surface irrigation water and seasonality on fecal hygiene indicator E. coli in leafy green production, Food Microbiology 75:82-89 (2018); Kundu et al., Quantitative microbial risk assessment to estimate the risk of diarrheal diseases from fresh produce consumption in India, Food Microbiology 75:95-102 (2018); Topalcengiz & Danyluk, Assessment of contamination risk from fecal matter presence on fruit and mulch in the tomato fields based on generic Escherichia coli population, Food Microbiology 103:103956 2022 (2022); Truchado et al., Suitability of different Escherichia coli enumeration techniques to assess the microbial quality of different irrigation water sources, Food Microbiology 58:29-35 (2016). Bacteroidales are a common target as they are confined to warm-blooded animals and are a major component of gut microflora. Bernhard & Field, A PCR assay to discriminate human and ruminant feces on the basis of host differences in Bacteroides-prevotella genes encoding 16S rRNA, Applied Environmental Microbiology 66 (10): 4571-4574 (2000).

Conventional laboratory procedures for FIB detection include culture-based methods and DNA-based approaches and usually require an enrichment step that takes several hours. Hoadley & Cheng, The recovery of indicator bacteria on selective media, J Applied Bacteriology 37 (1): 45-57 (1974); Li et al., Formation and Control of the Viable but Non-culturable State of Foodborne Pathogen Escherichia coli 0157-H7, Frontiers in Microbiology 11 (2020); Zhao et al., Current perspectives on viable but non-culturable state in foodborne pathogens, Frontiers in Microbiology 8 (2017).

Culture-dependent approaches, however, have many limitations including, for example, that overnight incubation is required, which can significantly delay findings and prevent early warnings and prompt implementation of contamination control or mitigation steps. To more quickly determine microbial contamination, molecular techniques such as PCR have been used. PCR-based approaches for monitoring FIB depend heavily on access to a laboratory, professional staff, and expensive equipment and, thus, are not conducive to rapid in-field assessment of contamination. Further, due to the low quantity of pathogen typically present, PCR techniques are likely to give a false negative result.

Compared to PCR, loop-mediated isothermal amplification (LAMP) enables simpler detection of microorganisms in environmental samples. Notomi et al., Loop-mediated isothermal amplification of DNA, Nucleic Acids Research 28 (12): e63 (2000). Due to the inherent characteristic of LAMP Bst DNA polymerase, only a single temperature (in the range of 60-65° C.) is required for the reaction to be conducted (as opposed to cycling of temperature, which is required for PCR). The reaction could be carried out in the field using a cost-effective, simple heat source, such as an incubator or a water bath, in contrast to the expensive thermocyclers needed by traditional PCR methods. Furthermore, the Bst polymerase is resistant to common PCR inhibitors found in unpurified environment samples, enabling direct measurements. As a result, LAMP has been widely used as a point-of-care assay for applications in food safety and diagnostics of human and animal health. Incorporating a colorimetric dye (e.g., EBT, phenol red) in LAMP assays enables color changes that are visible to the naked eye.

While LAMP and other isothermal amplification techniques do show promise as effective diagnostic tools, a major limitation has been the occurrence of false positives-either due to poor reagent handling or carryover contamination from previous experiments. Additionally, the accuracy of these techniques can be heavily dependent on the primers. Designing LAMP primers has proven challenging.

Accordingly, there remains a need to provide a cost-effective method to accurately characterize testbeds for distribution of aerosolized contamination with respect to, for example, crop fields. Ideally, the method should be easy to implement, available for in-field use, and provide clear and consistent results that are simple to interpret.

### SUMMARY

Rapid nucleic-acid biosensors are useful for on-farm detection of foodborne pathogens on fresh produce during pre-season and pre-harvest stages. Such tools aim to be user-friendly so that a producer could operate it in a few simple steps and detect multiple targets. Currently, an easy-to-use device for on-farm applications does not exist commercially. One of the bottlenecks is the delivery of a prescribed amount of sample to the reaction sites of the biosensor using a simple and precise approach. Here, drop dispensers using 3D-printing and a hydrophilic surface chemistry treatment to generate precise drops (DNA/bacterial samples) of a few micro-liters (˜20 to ˜33 μL) are provided. Multiple copies of these devices were tested over time of repeated application to estimate their shelf-lives which was, in certain embodiments, about 1 month (such as 1 month). In addition to drop generation tests, these devices were employed in nucleic-acid testing of fresh produce samples. The tests used loop-mediated isothermal amplification (LAMP) to detect DNA or whole cells of shiga-toxin producing Escherichia coli O157:H7 or DNA from Bacteroidales. These tests were performed to simulate the on-farm sample collection (using collection flags that we previously designed) and delivery using the drop dispensers. The results supported that these devices performed similarly to standard commercial pipettors in LAMP assays, providing a limit of detection of 7.8×10⁶ cell/mL for whole-cell detection of E. coli O57:H7 or 3 copies/cm² of Bacteroidales. This drop dispenser can also be used as part of a user-friendly consumable kit that can enable the performance of isothermal amplification assays on-site (e.g., on a fresh produce farm) by non-specialist users.

Also provided herein are methods for evaluating microbial contamination of an area of land. In certain embodiments, the methods for evaluating microbial contamination of an area of land comprise: obtaining a plurality of samples from various locations in an area of land of interest; detecting the presence or absence of a genetic target associated with contamination in each sample and, if the genetic target is present, quantifying a concentration of the contamination present in the sample; generating a heat map of the area of land from the detection and quantification data; collecting environmental data from the area of land over a period of time; and integrating the collected environmental data with the heat map to evaluate site-specific contamination risk in the area of land.

The contamination can comprise a fecal indicator bacteria (FIB). The FIB can be Bacteroidales, Escherichia coli, or Enterococcus faecalis.

In certain embodiments, obtaining a plurality of samples from various locations on an area of land comprises: positioning a plurality of collection flags for the collection of bioaerosol samples at various locations on the area of land, each collection flag comprising a film affixed to a support at a distance away from an end of the support such that, in use, the support can anchor the film a distance above a surface of an area in which the support is positioned; and collecting a sample from the film of each of the collection flags. The plurality of collection flags can comprise 1-200 collection flags per acre of the area of land. The plurality of collection flags can comprise 60 collection flags per acre of the area of land.

Collecting environmental data can comprise collecting weather data from one or more weather stations positioned on the area of land. The weather stations can be any weather stations or other weather data gathering devices known in the art. The environmental data can comprise temperature data, pressure data, humidity data, daily rain data, ultraviolet (UV) and solar radiation data, wind speed data, wind direction data, air quality data, or any combination of two or more of the foregoing.

Detecting the presence or absence of a genetic target associated with contamination in each sample can be performed using a polymerase chain reaction (PCR) assay or an isothermal amplification assay. Quantifying the presence or absence of a genetic target associated with contamination in each sample can further comprise: (1) providing at least one drop dispensing device comprising: a liquid holder that defines an interior, the interior in fluid communication with an inlet of the liquid holder and an outlet of the liquid holder, and a plunger configured to be movable up and down within at least a portion of the interior of the liquid holder such that downward movement of the plunger within the interior causes any fluid contained in the interior to be displaced through the outlet of the liquid holder, wherein the outlet of the liquid holder further comprises tip comprising a capillary tube that defines an inner surface in fluid communication with the interior; (2) providing at least one isothermal amplification assay device comprising two or more paper-based pads positioned in a stacked configuration relative to each other, at least one of the paper-based pads comprising a reaction pad loaded with one or more reagents comprising primer sets for the amplification of a genetic target associated with contamination, and at least one of the paper-based pads comprising a control pad that does not have amplification reagents thereon; (3) suspending a collected sample in a solvent housed within the liquid holder of the drop dispensing device; (4) loading the reaction pad of the assay device with the suspended sample by pressing the plunger of the drop dispensing device down to deliver at least a drop of the combined sample and solvent mixture to the reaction pad; and (5) heating the loaded reaction pad of the assay device to initiate amplification of the genetic target if present within the sample; and (6) detecting a visual result in the heated reaction pad indicative of the presence or absence of the contamination in the sample.

The primer sets of the assay device can be encoded by at least SEQ ID NOS: 7-27 and the genetic target is specific to Bacteroidales, and/or SEQ ID NOS: 1-6 and the genetic target is specific to Escherichia coli.

The heating step of the method can be performed for about 45 to about 120 minutes. Additionally or alternatively, the loaded reaction pad can be heated to a temperature of at or between 60-70° C. In certain embodiments, the loaded reaction pad is heated to a temperature of about 65° C. (such as 65° C.).

In certain embodiments, the method further comprises capturing at least one image of the heated reaction pad of the assay device and quantifying the concentration of the contamination in the sample from the at least one image. The area of land is in proximity to, or close proximity to, an animal operation.

The area of land can be a field, pre-plant or pre-harvest.

If, using the method, contamination is detected, the method can further comprise identifying a source of the contamination from the environmental data. There, the environmental data can be any of the environmental data described herein, for example, weather data (such as without limitation, wind data and/or humidity data).

In certain embodiments of the method, the heat map comprises a plurality of cells, each of the plurality of cells corresponding to an area in the area of land and associated with a value indicative of a concentration of the contamination in the corresponding area.

Methods for generating a heat map for a crop-field of interest are also provided. In certain embodiments, the methods for generating a heat map for a crop-field of interest comprises: obtaining a plurality of samples from various locations in a field of interest; detecting the presence or absence of a genetic target associated with contamination in each sample and, if the genetic target is present, quantifying a concentration of the contamination present in the sample; collecting environmental data from the field of interest over a period of time; and integrating the collected environmental data and the detection and quantification data to generate a heat map showing contamination in the field of interest.

The contamination density data of different regions in the heat map can be presented using color levels, for example. The contamination can be Bacteroidales. In certain embodiments, the contamination is E. coli and/or Bacteroidales. In certain embodiments, if contamination is detected, the method further comprises identifying a source of the contamination from the environmental data.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, figures, and tables.

FIG. 1A are cross-sectional diagrams depicting a drop dispenser device and operation thereof; FIG. 1B are photographs of a drop dispenser device and drop generation using the same; and FIG. 1C is a flow diagram of the post-fabrication surface treatment of a drop dispenser device by plasma and PEG 400 exposure.

FIG. 2A is a diagram of drop dispenser device design for variable volume dispensing capacity, including a description of the conical tip design for generation of micro-drops of different volumes; and FIG. 2B is a dispensing capacity map for drop dispenser devices with variable volumes for different tip angles.

FIG. 3 is a graph of comparison between the performance of some commercial pipettors versus the drop dispenser device (all values are in μL).

FIG. 4 is a calibration curve to estimate the E. coli O157:H7 cell counts from OD₆₀₀ reads.

FIGS. 5A and 5B relate to quantification of the E. coli O157:H7 DNA extract using PicoGreen dye, with FIG. 5A showing data correlating the Lambda DNA concentration with fluorescence intensity and FIG. 5B showing data related to estimating template DNA concentration.

FIGS. 6A and 6B relate to the effects of various surface treatment procedures on the dispensing capacity of the drop dispenser, with FIG. 6A showing photographs that depict the importance of having a hydrophilic interior and hydrophobic exterior surface for the liquid holder of the drop dispenser device on the dispensing performance, and FIG. 6B showing data related to the performance of drop dispensers treated by plasma and PEG 400 after storage. All values are in μL.

FIG. 7 shows data related to the effect of resin types on the performance of the drop dispensers. All values are in μL.

FIG. 8A is a comparison of the loop-mediated isothermal amplification (LAMP) assay results (replicate 1) when using the drop dispenser device versus a conventional Eppendorf 20-200 μL pipettor. A yellow color (lighter) indicates a positive result of the test and the boxes indicate the lowest concentration where all three replicates amplified (i.e., the limit of detection (LoD) for the experiment). The template was Shiga toxin (Stx)-producing E. coli (STEC) O157:H7 DNA extract at various dilutions.

FIG. 8B is a comparison of the LAMP assay results (replicate 1) when using the drop dispenser device versus a conventional Eppendorf 20-200 μL pipettor. A yellow color (lighter) indicates a positive result of the test and the boxes indicate the lowest concentration where all three replicates amplified (i.e., the LoD for the experiment). The template was E. coli O157:H7 DNA extract at various dilutions.

FIG. 8C is a comparison of the LAMP assay results (replicate 1) when using the drop dispenser device versus a standard Eppendorf 20-200 μL pipettor. A yellow (lighter) color indicates a positive result of the test and the boxes indicate the lowest concentration where all three replicates amplified (i.e., the LoD for the experiment). The template was E. coli O157:H7 DNA extract at various dilutions.

FIG. 9 is a comparison of the whole-cell LAMP assay results when using the drop dispenser device versus a standard Eppendorf 20-200 μL pipettor. A yellow (lighter) color indicates a positive result of the test and the boxes indicate the lowest concentration where all three replicates amplified (i.e., the LoD for the experiment). The template was E. coli O157: H7 cells at various dilutions.

FIG. 10 are the results of LOD tests for whole-cell LAMP assays using E. coli O157: H7 cells at various dilutions. A yellow color (lighter) indicates a positive result of the test and the boxes indicate the lowest concentration where all three replicates amplified (i.e., the LoD for the experiment). All liquid handling were performed using a standard Eppendorf 20-200 μL pipettor.

FIG. 11 is a schematic of the drop dispenser plunger 14 assembly (left) and a cross-sectional view (right) of the liquid holders of the drop dispenser device.

FIGS. 12A-12D are schematics representing design and workflow of an integrated paper-based testing system, with FIG. 12A showing a testing platform device comprising a heating unit, temperature control system, and an imaging unit, FIG. 12B showing a drop dispensing device for use with the testing system, FIG. 12C showing a LAMP assay device for use with the testing system, and FIG. 12D showing a workflow of the overall operation of the testing system where a user swabs a collection flag with a sterile swab, followed by resuspension of the swab in a solvent, and dispensation of a drop of the resuspended sample on μPADs (e.g., of an assay device) via a drop dispensing device. The μPADs are incubated at 65° C. for an hour, enabling LAMP amplification and a pH-induced color change from red to yellow in the presence of DNA synthesis. Post-reaction, the algorithm of the testing platform device can analyze the color of each μPAD to provide a quantitative readout (e.g., sample concentration in copies/μL).

FIG. 13 lists the sequences for primers and probes used in a LAMP reaction mix of Example 9 (Table 9), and additional LAMP primer sets.

FIGS. 14A-14D show data related to limit of detection (LoD) and quantitative analysis studies of LAMP assays. FIG. 14A is a photograph of endpoint (at 60 minutes) results of colorimetric LoD on paper using quantified swine stool DNA at the indicated concentrations (copies/reaction). The NTC replicates are reactions using nuclease-free water in lieu of DNA. FIG. 14B is a graph of quantitative analysis results of the LoD experiment of FIG. 14A using the image analysis algorithm of the platform device. The shaded area represents the standard deviations. FIG. 14C is a summary table of time to peak for the second derivative calculated from the results shown in FIG. 14B. The average of the three replicates was reported as x̅±relative standard deviations. FIG. 14D is a linear fit analysis generated by comparing the time-to-peak for the second derivative (FIG. 14C) and the concentration in logarithmic scaling. A regression line was generated to quantify the concentration of the reaction using sample's time-to-peak value.

FIG. 15 shows graphs of the second derivative (labeled R) of the positive percentage (labeled B) throughout the test run and plotted against time. The time-to-peak value for the second derivative was adopted as the indicator for sample DNA concentration.

FIGS. 16A-16C show mapping of risk of fecal contamination in a testing field. FIG. 16A is a satellite image of a sampled area. FIG. 16B shows risk of fecal contamination generated using paper LAMP assays as described in Example 11. Semi-quantitative analysis was performed on the paper LAMP assay result and the quantified target concentration was then converted into log₁₀ (copies/cm²). FIG. 16C shows risk of fecal contamination mapping using qPCR analysis. The Ct value of each qPCR reaction was converted to log₁₀ (copies/cm²) via a linear fit to log-transformed concentrations.

FIGS. 17A-17D are image analysis results from the field experiment of Example 11. FIG. 17A is a graph of the positivity percentage results over time for each field sample. The curves were smoothed using a moving average filter and plotted to display a qualitative analysis of the color change. FIG. 17B is a graph of the first derivative of the positive percentage in FIG. 17A. FIG. 17C is a graph of the positivity percentage result over time for each control sample. The curves were smoothed using a moving average filter and plotted to display a qualitative analysis of the color change. FIG. 17D is a graph of the first derivative of the positive percentage in FIG. 17C.

FIGS. 18A-18C show mapping results of the ASREC field with respect to fecal contamination studies. FIG. 18A is a satellite image of the sampled area. FIG. 18B shows the risk of fecal contamination generated using paper LAMP assay devices hereof form the results taken from the field shown in FIG. 18A. Semi-quantitative analysis was performed on the paper LAMP results, and the quantified target concentration was converted into log₁₀ (copies/cm²). FIG. 18C shows the risk of fecal contamination mapping using qPCR of the same field in FIG. 18A. The Ct value of each qPCR reaction was converted to log₁₀ (copies/cm²) via a linear fit to log-transformed concentrations.

FIGS. 19A and 19B are images of the same pad of an assay device hereof in adjacent timepoints with and without CLAHE. FIG. 19A is an image of a no-primer control pad at 22 minutes. FIG. 19B is an image of the same pad shown in FIG. 19A at 23 minutes. A significant color change can be seen between the two time points and is attributed to environmental factors (e.g., sunset) rather than the assay 200, which introduced a yellow filter that could interfere with image color analysis. The application of CLAHE effectively corrected the paper pad's color, resolving the interference of external environmental conditions.

FIGS. 20-25 show code that can be executed by a processor for analysis of images captured using the testing systems hereof and related protocols, with FIG. 20 showing code for [insert]; FIG. 21 showing code for a program that controls a heating element of the testing system to maintain a constant target temperature of 65° C. which is read by the temperature sensor of the temperature control unit of the system; FIG. 22 showing code for a program that graphs a specified number of equally spaced values to a sigmoid curve with a specific steepness of the curve parameter ‘k″; FIG. 23 showing code for a program for image preprocessing that was used on the ASREC field experiment before grabcut_rectangle was run (Example 11); FIG. 24 showing code for a program for use with grabcut_rectangle to quantify the precent positivity of each sample pad; and FIG. 25 showing a program code for use with filter_function to mask out the sample pads and quantify their percent positivity.

FIG. 26 illustrates the fabrication process of collection flags, with subpart A showing the starting materials; subpart B showing the cutting of transparent film to 5 cm×30 cm strips; subpart C showing four pieces of film being stapled together at the edge to form a loop; subpart D showing a bamboo skewer (support) being inserted through the loop to make a collection flag; and subpart E showing a completed collection flag ready for deployment.

FIGS. 27A and 27B show results of qPCR assays using lettuce leaf swab (FIG. 27A) and flag swab (FIG. 27B) resuspension solution. 1 μL of resuspension was added to the reaction mix in each case.

FIG. 28 shows images and heat maps related to background levels of fecal contamination around animal operations, with subparts A, B and C showing satellite images of the sampled area, and subpart D showing a heat map of the fecal contamination level in the sampled area using qPCR assays.

FIG. 29 shows risk of fecal contamination mapping using qPCR (May 2021). The Ct value of each qPCR reaction was converted to log₁₀ (copies/cm²) via a linear fit to log-transformed concentrations.

FIG. 30 shows risk of fecal contamination mapping using qPCR (August 2021). The Ct value of each qPCR reaction was converted to log₁₀ (copies/cm²) via a linear fit to log-transformed concentrations.

FIG. 31 is a scatter plot of microbial source tracking results. Each of the 17 samples with a concentration of greater than 1 copy/reaction was selected for microbial source tracking. The experiment was carried out in a dPCR method. Each sample was tested with four different host-specific qPCR primer-probe sets (cattle-specific Bacteroidales, swine-specific Bacteroidales, human-specific Bacteroidales, and poultry-specific Bacteroidales). On the scatter plot, the fluorescence intensity of each partition was displayed. Based on the NTC, the fluorescence threshold was determined to be 20 RFU.

FIGS. 32A and 32B are plots of quantified background levels of fecal contamination with FIG. 32A for fresh pack lettuce and FIG. 32B for processed lettuce harvester samples, using the lab run qPCR data.

FIG. 33 are graphs of data obtained from two different weather stations to compare the PM2.5 (top) and wind speed (bottom) measurements.

FIGS. 34A-34C are related to preliminary qpCR analysis of collection flags in a stone fruit farm. FIG. 34A is a map of sites for the installation of flags, with points in the panel representing the locations where flags were located. FIGS. 34B and 34C are qPCR heat maps from small flag and large flag samples, respectively.

FIG. 35 are a satellite image (left) and a schematic (right) of a swine operation at ASREC, where each dot labeled “WS-[number]” represents a weather station and each unlabeled dot represents 10 flags (with a total of 120 flags placed). 12 flags were collected weekly over the course of a 10-week study.

FIG. 36 are heat maps with wind data taken over a 10-week study (panel A), with PM2.5 data (panel B), and with PM10 data (panel C). The size of the oval shape symbol represents the amount of PM2.5 while the size of the triangle symbol represents the amount of PM10. The heat map of Bacteroidales concentration shows bioaerosol distribution of fecal matter. The length of the arrow and direction of point denote wind speed and direction, respectively.

FIG. 37 is wind data with a heat map of the distance extending from the swine operation to the field. The darkest boxes on the heat map denote missing collection flags. The map integrates the PM2.5 and PM10 data.

FIGS. 38A and 38B show a simplified map and an integrated heat map, respectively, of Bacteroidales concentrations across a farm in California integrated with weather data.

FIG. 39 is a simplified map of an Arizona farm location, along with heat maps showing log₁₀ (copies/cm²) levels for the samples collected. Wind and PM2.5 and PM10 data are integrated.

FIG. 40 shows simplified maps of a lettuce field in Arizona along with heat maps showing log₁₀(copies/cm²) levels for the samples collected.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purpose of promoting an understanding of the principles of the present disclosure, reference will now be made to the embodiments illustrated in the drawings, tables, and figures and specific language will be used to describe the same. It will nevertheless be understood that no limitation of the scope of this disclosure is thereby intended.

The present disclosure provides methods for determining or characterizing the distribution of microbial (e.g., fecal) contamination in a field used, or to be used, to grow fresh produce. As used herein, the term “fresh produce” includes both cut and whole fresh fungi, fruits, and vegetables including, for example and without limitation, greens, celery, berries, and the like. The term “fresh” means that the food is in its raw state and has not been frozen or subjected to any form of thermal processing or any other form of preservation (other than potentially post-harvest pesticides, the application of a mild chlorine wash or mild acid wash, or treatment with ionizing radiation). The methods integrate novel devices, systems, and methods of detecting the presence or absence of contamination in a fresh produce crop and/or field containing the same, and data (including, for example, metadata) collected from weather stations to generate a risk heat map for fresh produce operations. A heat map can be, for example, a method for visualization of 2D contamination density.

These methods can be particularly advantageous for fresh produce operations that are in close proximity with animal operations (i.e., those that are “high risk” for fecal contamination). “High-risk” as used herein means a target that measures as having a high concentration of fecal indicator bacteria (FIB) (e.g., at or about 4 orders of magnitude higher than a “low-risk” threshold) and, thus, is contaminated with fecal matter and foodborne pathogens. “Low-risk” as used herein means a target that measures as having a low concentration of FIB and, thus, is not likely contaminated with feces and/or foodborne pathogens to the extent fresh produce grown therein would result in consumer illness. In certain embodiments, a “low-risk” threshold is the targeted FIB being present in at or less than 2 copies/cm² of surface area on the collection surface. In certain embodiments, a “low-risk” threshold is the targeted FIB being present in at or less than 10 copies/cm² of surface area on the collection surface. In certain embodiments, the “low-risk” threshold is the FIB being present in less than 17 copies/cm² of surface area on the collection surface (e.g., 16 copies/cm², 15 copies/cm², 15 copies/cm², 14 copies/cm², 13 copies/cm², 12 copies/cm², 11 copies/cm², 10 copies/cm², 9 copies/cm², 8 copies/cm², 7 copies/cm², 6 copies/cm², 5 copies/cm², 4 copies/cm², 3 copies/cm², 2 copies/cm², 1 copies/cm², or less than 1 copies/cm²).

To assess the level of fecal contamination in water, it is common practice to quantify the abundance of one or more FIBs. Brauwere & Servais (2014), supra. FIBs, such as Escherichia coli, Enterococcus faecalis, and Bacteroidales, have been selected as potential indicators of fecal contamination. Allende et al. (2018), supra; Denis et al. (2016), supra; Tambalo et al., Persistence of host-associated Bacteroidales gene markers and their quantitative detection in an urban and agricultural mixed prairie watershed, Water Research 46 (9): 2891-2904 (2012). Bacteroidales are particularly useful because they are restricted to warm-blooded animals and are a key component of gut microflora. Bernhard & Field (2000), supra. Further, as obligate anaerobes, Bacteroidales are unable to proliferate in standard atmospheric conditions, making it less likely they would be overrepresented in assessments of fecal contamination.

The methods hereof use a combination of in-field FIB measurements and weather data to assess the risk of general fecal contamination and for microbial source tracking. In certain embodiments, a risk rating can be calculated for specific fresh produce operations and/or fields, and such risk can be tracked over time.

These methods hereof leverage devices and systems (and methods of detecting and/or treatment using such devices and systems) that are portable, user-friendly, disposable, and capable of providing fast and accurate results in the field without the need for a laboratory and other complex equipment. Such devices, systems, kits, and methods can be operated/performed in a few simple steps by a user with minimal training and can detect multiple targets with a high degree of accuracy. In certain embodiments, such methods can utilize a drop dispensing device, one or more assay devices, and/or systems comprising the foregoing. Each of these components will be described in detail below, followed by a detailed description of the methods.

### Drop Dispensing Devices

The drop dispenser device can comprise a hydrophilic surface chemistry treatment to facilitate the generation of precise drops (e.g., of DNA/bacterial samples) of a few micro-liters (for example, about 20 μL to about 33 μL). Such devices can be used in a system (e.g., with the assay device) that leverages LAMP to detect a genetic target associated with contamination (e.g., DNA or whole cells of bacteria or other pathogens). Such tests can be performed on-farm following sample collection (e.g., using collection flags or otherwise) and samples can be delivered to the assay using the drop dispenser devices hereof.

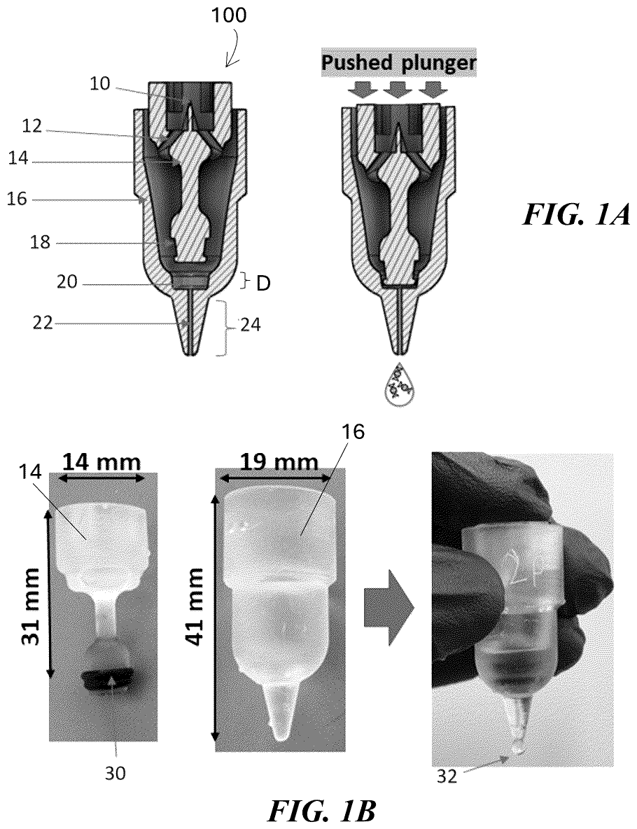

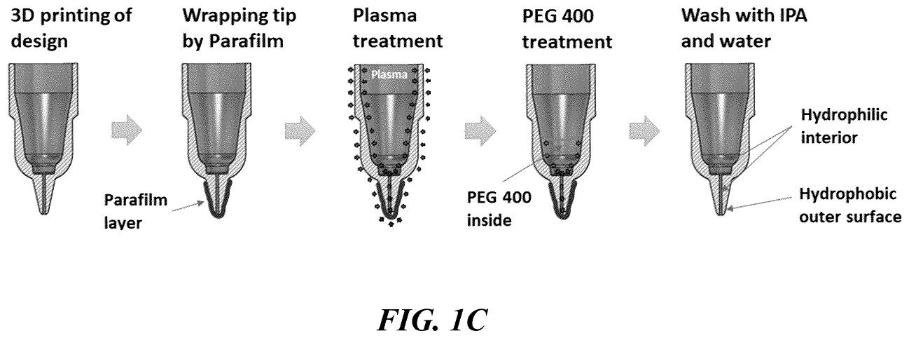

Now referring to FIG. 1A, a drop dispensing device 100 is shown. The drop dispensing device 100 can comprise two main portions: a plunger 14 and a liquid holder 16. At least a portion of the drop dispensing device 100 (e.g., the plunger 14, the liquid holder 16, or both the plunger 14 and liquid holder 16) can be formed from a resin and/or manufactured via 3D printing. In certain embodiments, the resin can be or comprise High Temp V2, Rigid 4000 V1, BioMed Clear V1, or Clear V4. It will be understood that this is not an exhausted list of resins that may be employed for the manufacture of all or a portion of the device 100 and, similarly, many other resins and/or combinations of resins can be used. In certain embodiments, one or more of the portions of the drop dispensing device 100 can be formed by injection molding. Fabrication of injection molded drop dispensing devices 100 can include sonic welding or ultraviolet (UV)-curing glues for assembly of parts, for example.

The liquid holder 16 of the device 100 defines an interior and can comprise a liquid inlet 12 and/or an outlet 20, both in fluid communication with the interior of the liquid holder 16. The device 100 can further comprise an opening on an exterior surface thereof (not shown) that is in communication with the interior of the liquid holder 16. The opening can be for receipt of a sample therethrough and may be sealed to prevent contamination or leakage of any solvent contained within the interior of the liquid holder 16. In certain embodiments, the opening comprises a one-way valve configured to receive a swab capture device therethrough. Swab capture devices are a means for sanitary sample capture. Accordingly, where the opening comprises a one-way valve, the swab to be analyzed is inserted into the interior of the liquid holder 16 of the device 100 through the opening and the handle of the swab can then be broken off so that the swab becomes sealed inside the device 100. Many one-way valves, caps, and other opening designs are well known in the art and can be applied in the present context. The interior of the liquid holder 16 can be preloaded with a solvent such as water, a buffer, or the like that is suitable to resuspend a collected sample deposited within the liquid holder 16.

The diameter of the interior can be, at its widest point, at or between 10 mm-25 mm, at or between 11 mm-24 mm, at or between 12 mm-23 mm, at or between 13 mm-22 mm, at or between 14 mm-21 mm, at or between 15 mm-20 mm, at or between 16 mm-19 mm, or at or between 17 mm-18 mm (wherein the stated ranges include the stated end points all 1 mm increments encompassed thereby).

The drop dispensing device 100 can be single entry. “Single entry” devices are disposable, and intended for single use. Generally, one sample per device is applied, the device is then optionally sealed, and the drop functionality performed.

The device 100 can further comprise a discharge insert positioned within the interior of the liquid holder 16 and in fluid communication with the outlet 20. The discharge insert can comprise a liquid inlet 12 configured to receive fluid into an interior of the discharge insert, for example, when the plunger 14 is pushed down. The discharge insert can comprise a depth D, the parameters of which can be adjusted to achieve a particular drop volume. In certain embodiments, the discharge insert comprises at least a portion having a cylindrical shape (i.e., comprises a discharge cylinder).

The liquid inlet 12 can optionally comprise a membrane tearing tip 10. The membrane tearing tip 10 can comprise a shaped surface at or surrounding the inlet 12 and a film, membrane, or other barrier) that extends across the liquid inlet 12 and seals the same. In operation, when the plunger 14 is depressed, the plunger 14 seats against the membrane tearing tip 10 thereby puncturing or tearing the film, membrane or other barrier and allowing flow through the inlet 12 and into the discharge portion (FIG. 1A). In this manner the membrane tearing tip 10 can facilitate separation between the interior of the discharge portion of the device 100 and the interior of the liquid holder 16 until drop generation is desired. This can further facilitate resuspension or storage of a sample within a solvent contained in the interior of the liquid holder 16 without the risk of leakage through the outlet 20.

The outlet 20 can be an opening within the liquid holder 16 that is fluid communication with the interior of the liquid holder 16 via the interior of the discharge insert.

In certain embodiments, the drop dispensing device 100 further comprises at least one O-ring seat 18 formed or defined within a wall of the discharge insert. Each O-ring seat 18 can be formed to receive an O-ring 50 therein. In operation, when the plunger 14 is pushed down/depressed, the O-ring(s) can seal the interior of the discharge insert from the interior of the liquid holder 16.

In certain embodiments, the outlet 20 further comprises a tip 24 extending from the discharge insert. The tip 24 can comprise an inner surface (e.g., in fluid communication with the interior of the liquid holder 16) and an outer surface. The tip 24 can comprise a capillary tube 22, wherein the capillary tube 22 defines the inner surface. The inner surface of the tip 24 can be hydrophilic. The outer surface of the tip 24 can be hydrophobic. In certain embodiments, the inner surface of the tip 24 is hydrophilic and the outer surface of the tip 24 is hydrophobic. The tip 24 can define a length and a diameter. The parameters of the tip 24 length and/or diameter can be modified to achieve a desired drop volume.

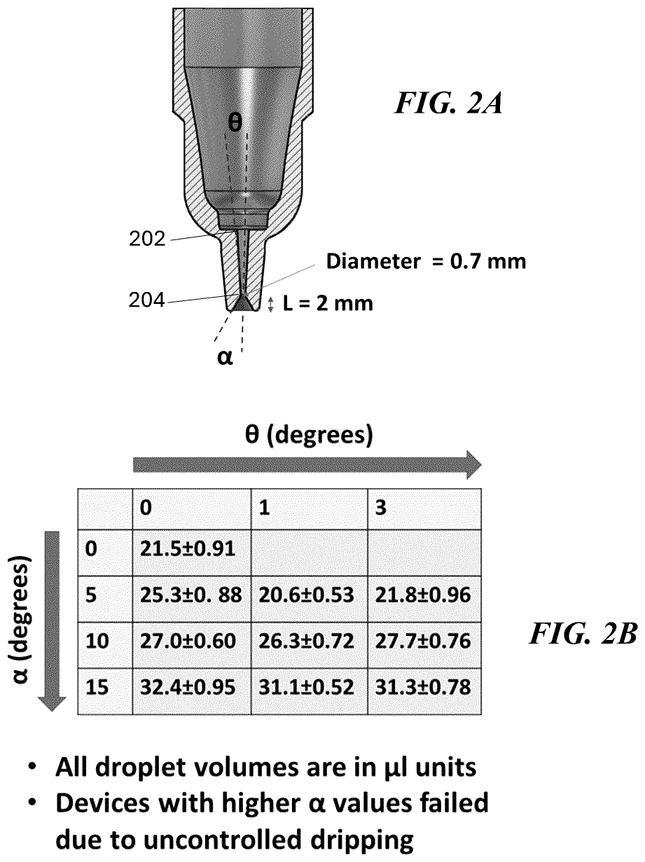

As shown in FIG. 2A, the tip 24 can further define a first angle θ along a length of the tip and/or a second angle α at a distal end of the tip 24. Where the tip 24 defines a first angle θ, the diameter of the tip 24 can narrow from a wider proximal tip portion 202 to a narrower section of the distal tip portion 204. The first angle θ can be at or between 0-1 degrees, at or between 1-2 degrees, at or between 2-3 degrees, or at or between 2-5 or 2-10 degrees (all ranges inclusive of the stated endpoints and all 0.1 degree increments encompassed thereby). In certain embodiments, the first angle θ is 1 degree. In certain embodiments, the first angle θ is 2 degrees. In certain embodiments, the first angle θ is 3 degrees.

Where the tip 24 defines a second angle α, the distal tip portion 204 can further comprise an angled outlet as shown in FIG. 2A. Such angled outlet may comprise a wider diameter than the narrower section of the distal tip portion 204 and/or can define a length L. The second angle α can be at or between 0-25 degrees, at or between 1-24 degrees, at or between 2-23 degrees, at or between 3-22 degrees, at or between 4-22 degrees, at or between 5-21 degrees, at or between 6-20 degrees, at or between 7-19 degrees, at or between 8-18 degrees, at or between 9-17 degrees, at or between 10-16 degrees, at or between 11-15 degrees, or at or between 12-14 degrees (all ranges inclusive of the stated endpoints and all 0.1 degree increments encompassed thereby). In certain embodiments, the second angle α is 0 degrees (i.e., straight). In certain embodiments, the second angle α is 5 degrees. In certain embodiments, the second angle α is 10 degrees. In certain embodiments, the second angle α is 15 degrees. In certain embodiments, the second angle α is 20 degrees.

In certain embodiments, the plunger 14 is configured to move up and down within at least a portion of the interior of the liquid holder 16. When seated within at least a portion of the interior of the liquid holder 16, the plunger 14 can displace any fluid held therein through the outlet 20 of the device 100.

In certain embodiments, the plunger 14 can seat against at least one O-ring 50 positioned within the seat 18. For example, in operation, when the plunger 14 is pushed down, the O-ring(s) 50 can seal the discharge insert/outlet 20 from the liquid holder 16. Further downward movement of the plunger 14 can cause liquid within the outlet 20 (e.g., discharge insert and tip 24) to be dispensed out. At this stage, the device 100 can act as a positive displacement pump.

The drop dispensing device 100 can optionally further comprise a compression spring installed at a neck of the plunger 14 to facilitate the back movement of the plunger 14 after its release. In certain embodiments, the compression spring can comprise a 0.6 mm×9.5 mm×20 mm spring.

The drop dispenser device 100 can be subjected to one or more surface treatments to, for example, enhance the reliability of the drop dispenser device 100 (as compared to an untreated device). Such surface treatments can include, without limitation, exposure to plasma for a specified duration (e.g., 1 minute, 2 minutes, 3 minutes, 4 minutes, 5 minutes, 6 minutes, or less than 10 minutes). In certain embodiments, the surface treatment comprises exposure to plasma for a specified duration, followed by exposure to polyethylene glycol (PEG) 400 for 24 hours after plasma treatment by either dipping the device 100 in the PEG 400 or pouring 600 μL of PEG 400 into the interior of the device 100. FIG. 1C illustrates an embodiment of the latter approach, where the tip 24 is wrapped with multiple layers of Parafilm wrapping film before exposure to plasma and PEG 400. The plasma treatment can be performed using an air plasma generator, for example, which can be advantageous to keep the outer tip surface hydrophobic, but can result in the inner surface of the tip 24 being hydrophilic. After surface treatment, the assembled drop dispenser device 100—including the plunger 14, O-rings 50, and the liquid holder 16, for example—can be thoroughly washed with isopropyl alcohol (IPA) and/or rinsed with ultra-pure water to remove any residue that could interfere with LAMP reactions.

The liquid holder 16 of the device 100 can be pre-filled with media (or solvent) for preserving and/or suspending a sample. In use, a user can insert a sample (e.g., using a swab or other collection device) into the opening of the liquid holder 16 as described above.

The range of drop volume that could be generated using our devices, while maintaining an acceptable precision, was between about 20 μL to about 33 μL (such as about 20 μL to 33 μL, 20 μL to about 33 μL, or 20 μL to 33 μL). In certain embodiments, the drop volume is between about 21 μL to about 32 μL (such as about 21 μL to 32 μL, 21 μL to about 32 μL, or 21 μL to 32 μL). In certain embodiments, the drop volume is between about 22 μL to about 31 μL (such as about 22 μL to 31 μL, 22 μL to about 31 μL, or 22 μL to 31 μL). In certain embodiments, the drop volume is between about 23 μL to about 30 μL (such as about 23 μL to 30 μL, 23 μL to about 30 μL, or 23 μL to 30 μL). In certain embodiments, the drop volume is between about 24 μL to about 29 μL (such as about 24 μL to 29 μL, 24 μL to about 29 μL, or 24 μL to 29 μL). In certain embodiments, the drop volume is between about 25 μL to about 28 μL (such as about 25 μL to 28 μL, 25 μL to about 28 μL, or 25 μL to 28 μL). In certain embodiments, the drop volume is between about 26 μL to about 27 μL (such as about 26 μL to 27 μL, 26 μL to about 27 μL, or 26 μL to 27 μL). In certain embodiments, the device 100 can deliver drops with consistent 27 μL volumes.

Methods of manufacturing and/or fabricating the drop dispensing device 100 are also provided. In at least one embodiment, a method of manufacturing the drop dispensing device 100 comprises 1) forming a plunger 14, and 2) forming a liquid holder 16 configured to couple with (or receive within an interior defined by the liquid holder 16) the plunger 14.

In certain embodiments, the method comprises forming, using 3D printing, injection molding, or extrusion molding, a liquid holder 16 (e.g., any of the liquid holders of the devices described herein). The liquid holder 16 can, for example, comprise an inlet 12, an outlet 20, and an interior in fluid communication with the inlet 12 and outlet 20. The outlet 20 of the liquid holder 16 can further comprise a tip 24 comprising a capillary tube 22 that defines an inner surface in fluid communication with the interior of the liquid holder 16. The method can further comprise forming, using 3D printing, injection molding, or extrusion molding, a plunger 14 configured to be seated within, and movable up and down within, at least a portion of the interior of the liquid holder 16; and exposing at least the liquid holder 16 (and, optionally, the plunger 14) to one or more surface treatments. The liquid holder 16 and/or plunger 14 can be formed from High Temp V2, Rigid 4000 V1, BioMed Clear V1 or Clear V4 resins. The one or more surface treatments can result in at least the interior walls of the liquid holder and inner surface of the capillary tube 22 being hydrophilic and an outer surface of the tip 24 being hydrophobic. In certain embodiments, the one or more surface treatments result in the plunger 14 comprising a hydrophobic outer surface.

The one or more surface treatments can comprise exposure of the liquid holder 16 (and/or plunger 14) to plasma for curing, during or after the forming process. The one or more surface treatments can comprise exposure of the liquid holder 16 (and/or plunger 14) to a chemical treatment, during or after the forming process. The one or more surface treatments can comprise exposure of the liquid holder 16 (and/or plunger 14) to PEG, during or after the forming process. In certain embodiments, the one or more surface treatments comprises exposing at least the liquid holder 16 (and/or plunger 14) to plasma for curing, during or after the forming process; and exposing the plasma cured liquid holder 16 (and/or plunger 14) to PEG.

The plasma treatment can be performed using an air plasma generator, for example. Exposure to the plasma can be for a specified duration. The specified duration can be for 1 minute, 2 minutes, 3 minutes, 4 minutes, 5 minutes, 6 minutes, or less than 10 minutes, or between about 30 seconds and 10 minutes, between about 1 minute and 9 minutes, between about 2 minutes and 8 minutes, between about 3 minutes and 8 minutes, between about 4 minutes and 7 minutes, or between about 5 minutes and 6 minutes (the stated ranges include all specified endpoints and 10 second increments encompassed thereby). In certain embodiments, the exposure to plasma is for 1 minute. In certain embodiments, the exposure to plasma is for 2 minutes. In certain embodiments, the exposure to plasma is for 3 minutes.

Exposure to PEG can be achieved by dipping the liquid holder 16 (and/or plunger 14) into a PEG solution or pouring a PEG solution into the interior of the liquid holder 16 (and/or over the plunger 14). In certain embodiments, 600 μL of PEG 400 is poured into the interior of the liquid holder 16 or over the plunger 14. The PEG can be any suitable PEG. The PEG can be PEG 400. The exposure to PEG can be for a specified duration. For example, and without limitation, the exposure to PEG can be for at or between 1-36 hours, at or between 2-36 hours, at or between 3-35 hours, at or between 4-34 hours, at or between 5-33 hours, at or between 6-32 hours, at or between 7-31 hours, at or between 8-30 hours, at or between 9-29 hours, at or between 10-28 hours, at or between 11-27 hours, at or between 12-26 hours, at or between 13-25 hours, at or between 14-24 hours, at or between 15-23 hours, at or between 16-22 hours, at or between 16-21 hours, at or between 17-20 hours, or at or between 18-19 hours (wherein the specified ranges are inclusive of the stated endpoints and all 10 minute increments encompassed thereby). In certain embodiments, the exposure to PEG is for 20 hours. In certain embodiments, the exposure to PEG is for 22 hours. In certain embodiments, the exposure to PEG is for 24 hours. In certain embodiments, the exposure to PEG is for 26 hours.

In certain embodiments, the one or more surface treatments comprise exposure to plasma for a specified duration, followed by exposure (e.g., immediate or near-immediate exposure) to PEG 400 for 24 hours by either dipping the device 100 in the PEG 400 or pouring a PEG solution into the interior of the device 100.

The method can further comprise wrapping the tip 24 of the liquid holder 16 in wrapping film prior to exposing the liquid holder 16 to the one or more surface treatments. In certain embodiments, for example, the tip 24 can be wrapped with one or more layers of Parafilm wrapping film before exposure to plasma and/or PEG.

After surface treatment(s), the assembled drop dispenser device 100—including the plunger 14, O-rings 50, and the liquid holder 16, for example—can be thoroughly washed with isopropyl alcohol (IPA) and/or rinsed with ultra-pure water to remove any residue that could interfere with LAMP reactions.

### Assay Devices

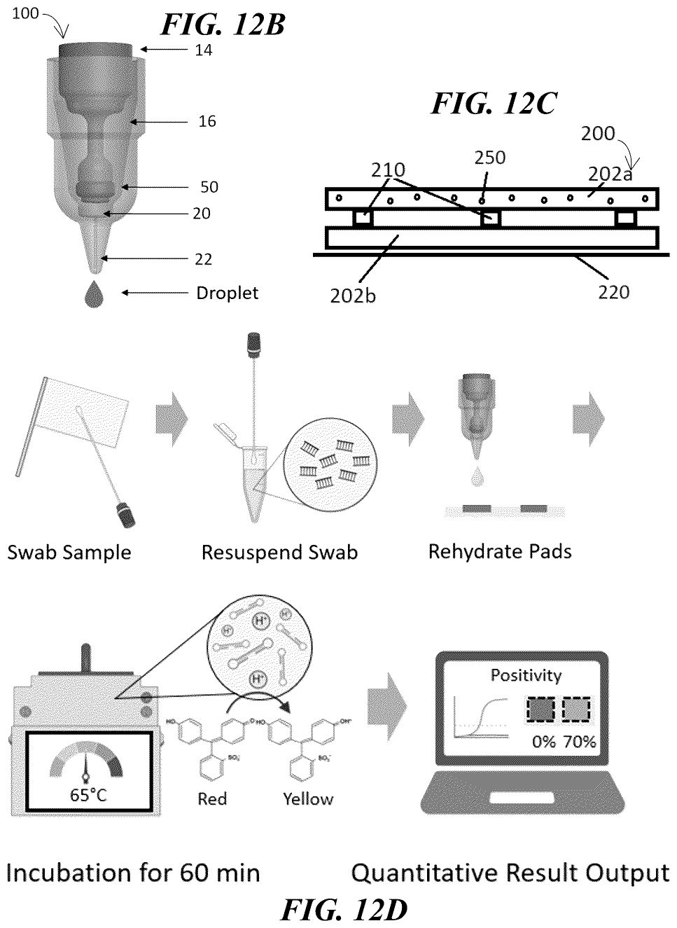

As noted above, assay devices 200 are also provided. FIG. 12C shows a schematic of an embodiment of a assay device 200. The assay device 200 can be paper-based and/or microfluidic and comprise a specialized colorimetric isothermal reaction mix of reagents 250 (e.g., a LAMP reaction mix). The assay device 200 can be configured to allow for a simple fabrication process.

As used herein, a “microfluidic device” is a hydraulic device, cartridge, or card with at least one internal channel, void or other structure that has at least one dimension smaller than about 500 microns, but in some cases as much as twice that if desired. The assay devices 200 can be hybrids of microfluidic and microscale fluid structures, but generally require small sample volumes of less than 1 mL, such between about 10 μL-about 200 μL, between about 20 μL-about 180 μL, between about 30 μL-about 160 μL, between about 40 μL-about 160 μL, between about 60 μL-about 140 μL, or between about 80 μL-about 120 μL (wherein the specified ranges are inclusive of the stated endpoints and all 1 μL increments encompassed thereby). In certain embodiments, the sample volume is at or about 25 μL, 26 μL, 27 μL, 28 μL, 29 μL, or 30 μL. Microscale is taken to indicate an internal dimension of less than about 5 mm, but it can be less than about 2 mm. On-board processing means for fluidic operations of pumping, diluting, concentrating, dissolving, diffusing, mixing, reacting, precipitating, adsorbing, filtering, lysing, separating, metering, heating, cooling, and condensing as are known in the art can be incorporated into the assay device 200.

The assay devices 200 can be fabricated from various materials using techniques such as laser stenciling, embossing, stamping, injection molding, extrusion molding, masking, etching, and three-dimensional soft lithography.

In certain embodiments, the assay device 200 is paper-based and can comprise two or more paper-based pads 202 coupled together via an adhesive or other non-adhesive binding. A pad 202 can comprise chromatographic paper, filter paper, cotton, nitrocellulose, and/or cellulose acetate. A pad 202 can be composed of a bibulous material, i.e., those a material that absorbs aqueous solutions by capillary action, as are well known in the art. One or more of the pads 202 can be microfluidic and/or comprise one or more microfluidic channels.

“Microfluidic channel,” or a “microchannel” means a fluid channel having variable length, but cross-sectional area often less than 500 μm, in some cases twice that. Microfluidic fluid flow behavior in a microfluidic channel is highly non-ideal and laminar, as in Poiseuille flow, and can be more dependent on wall wetting properties and diameter than on pressure drop.

The pad 202 can be self-priming. As used herein, “self-priming” means a channel (e.g., a microfluidic channel) that is fabricated from a material or is treated so that the channel is wettable and capillary flow begins generally without the need to prime the channel.

In certain embodiments, the assay device 200 comprises a first pad 202a and a second pad 202b positioned in a stacked configuration relative to each other. Each of the pads 202 can comprise 5 mm×6 mm chromatography attached together via a double-sided adhesive, for example. At least one of the pads 202 comprises a reaction pad that is seeded or pre-loaded with one or more reagents 250, and at least one of the pads is a non-primer, control pad. The control pad can serve as a negative control that, in use, can be used to confirm that any positive result obtained from the reaction pad is indicative of the presence of one or more targets and not influenced by factors such as changes in sample pH or issues related to reagent storage. Davidson et al., A paper-based colorimetric molecular test for SARS-COV-2 in saliva, Biosensors & Bioelectronics: X 9:100076 (2021).

The reagents 250 preloaded on the reaction pad 202 comprise one or more amplification reagents that, when used with the isothermal amplification methods such as LAMP, enable single-temperature operations that can be performed with simple equipment such as a water bath or incubator.

In certain embodiments, the reagents 250 comprise one or more of the primer sets described in International Patent Application Publication No. WO 2023/225573, the contents of which are incorporated herein by reference in their entirety. In certain embodiments, the reagents 250 comprise a LAMP reaction mix reported in Wang et al., Fabrication of a paper-based colorimetric molecular test for SARS-COV-2, MethodsX 8:101586 (2021), which is a mix of colorimetric reagents that can provide immediate or near-immediate feedback on the detection of specific analytes by exhibiting a color change.

The reagents 250 can be selected to target different genetic targets associated with contamination (e.g., pathogenic agents, FIB, or other targeted organisms) in a sample. In certain embodiments, the reagents 250 are a LAMP reaction mix that comprises a LAMP primer set based on the conserved region of the 16S ribosomal RNA (16S rRNA) gene that targets Bacteroidales. Accordingly, the reagents 250 can comprise a LAMP primer set that targets DNA fragments specific to Bacteroidales. In certain embodiments, the LAMP reaction mix comprises primers encoded by SEQ ID NOS: 7-27. The reagents 250 can comprise a LAMP primer set that targets DNA fragments specific to E. coli. In certain embodiments, the LAMP primer set that targets DNA fragments specific to E. coli and comprises primers encoded by SEQ ID NOS: 1-6.

The reagents 250 can further comprise an indicator associated with each primer set thereof. More specifically, the primer sets of the reagents 250 can be configured to include the indicator (e.g., a fluorescent indicator coupled with an end of each primer) or the indicator can be added to any media housed within the liquid holder 16 of the drop dispensing device 100 or to the reaction pad 202 itself. The indicator can be any indicator suitable for use with the primer sets.

In certain embodiments, the reagents 250 comprise both colorimetric and fluorometric dual-signal reagents. In certain embodiments, the reagents 250 comprise fluorescent reporters. In certain embodiments, the reagents 250 comprise colorimetric reporters.

The device 200 can comprise a landing area into which a sample to be tested can be deposited. The landing area is in fluid communication with a testing site (i.e., location of the LAMP reaction mix) of the reaction pad 202. The reagents 250 can be localized in a testing site location of the reaction pad 202.

The reagents 250 can be integrated into (e.g., pre-loaded or seeded onto) the reaction pad 202 using methods known in the art. For example, prior to assembly, the reagents 250 can be applied in or on the reagent pad 202 (e.g., in or on microfluidic channels thereof and/or in the test field) in a variety of ways. Various “printing” techniques are suitable for application of liquid reagents to layers of the device 200 (e.g., micro-syringes, pens using metered pumps, direct printing, ink-jet printing, air-brush, contact (or filament) methods, and the pads 202 and other components are then assembled into the completed assay device 200. In certain embodiments, the reaction pad 202 contains on-boarded reagents 250 sufficient for the analysis of a single test specimen.

An optical window can also be provided with a view of the testing site such that a user can visualize the results of a reaction with the reagents 250. Masking can be used, as desired, to define boundaries within which the reagents 250 are loaded. Masking to mark out a test site can aid in visual recognition of a positive assay and also in machine-aided image analysis of automated test results. Plastic surfaces can be passivated outside the defined boundaries of the mask, or in negative masking techniques, the plastic surface will be activated, such as by low pressure gas plasma treatment, where unmasked.

The assay device 200 can further comprise one or more spacers 210 positioned between two or more of the pads 202 to provide space therebetween. A spacer 210 can comprise a solid phase substrate such as a plastic. A spacer 210 can comprise a material selected from the group consisting of polystyrene, polycarbonate, polypropylene, polyethylene terephthalate (PET), and polyamide. The spacer 210 can comprise any suitable dimensions. The spacer 200 can comprise a 5 mm spacer. The spacer 200 can comprise a 3 mm spacer. The spacer 200 can comprise a 7 mm spacer. The spacer 200 can comprise a 10 mm polystyrene spacer. The spacer 200 can comprise a 5 mm polystyrene spacer.

Optionally, the stack of pads 202 can be coupled with a support 220 (e.g., a backer) to provide support to the overall assay device 200. A support 220 can be composed of one or more polymers such as polyethersulfones, polyesters, poly(vinyl chloride), vinyl chloride-propylene copolymer, latex, vinyl chloride-vinyl acetate copolymer, PET, polyproplyene, polystyrene, polycarbonate, polyacrylamides, polyacrylates, polyamides, nylons for example, wettable polyvinylidene fluoride (PVDF), either used as supplied or in composites with other materials; or glass, silica, ceramic materials or exploded metals. The support 220 can be rigid or semi-rigid to act as a backing for the assay device 200. The pads 202 can be coupled with the support 220 via an adhesive or other bonding technique. In certain embodiments, at least one of the pads 202 is attached via a double-sided tape to the support 220.

In certain instances, the assay device 200 can be laminated and fabricated with one or more adhesive interlayers or by thermal adhesive-less bonding techniques, such as by pressure treatment of oriented polypropylene. Fabrication of injection molded assay devices 200 can include sonic welding or UV-curing glues for assembly of parts (e.g., pads 202 and/or support 220).

For operation of the fully assembled assay device 200, a sample can be pipetted or otherwise deposited (e.g., using the drop dispensing device 100) into the device 200 and, optionally, sealed with a sanitary closure such as a cap, lid, or tape. The sample is introduced to the reagent pad 202, either directly when deposited thereon or via flow through the pad(s) 202 to the test site of the device 200. The sample loaded on the assay device 200 then interacts with the reagents 250 and detection of results (related to the presence or absence of one or more targeted pathogens or FIB within the sample) can be evaluated by an observer visually in a test field. Additionally or alternative, the results can be captured and evaluated by a machine equipped with an image capture device (e.g., a camera, spectrophotometer, a fluorometer, a luminometer, a photomultiplier tube, a photodiode, or the like). Following image capture via the image capture device, for example, a series of image processing techniques can be employed to ascertain the sample's result, either positive or negative, and the concentration of the target pathogen or FIB can be determined if a positive result is indicated.

In certain embodiments, the assay device 200 is intended for single use followed by disposal. Indeed, the assay device 200 can be single entry.

The assay device 200 can achieve a sensitivity of approximately 3 copies of Bacteroidales per cm² of a collection site's surface area (corresponding to 100 copies/reaction). In comparison to liquid assays (LoD 50 copies/reaction), the assay devices 200 exhibit a slightly worse LoD; however, due to a higher sample volume being added to the assay device 200 (adding 27 μL for assay device 200 versus 1 μL for liquid LAMP studies), its final LoD in copies of Bacteroidales per cm² of a collection site's surface is marginally better than the liquid assay (13.5x more sensitive). In addition, the multiple liquid handling steps and low volume pipetting (0.5-2.2 μL) necessary for the liquid LAMP assay renders it impractical for non-specialist users. In contrast, the assay devices 200 hereof have comparable sensitivity and represent a more viable option for on-field testing scenarios.

### Testing Systems & Kits

Systems and kits for testing one or more samples are also provided. Such testing systems and kits can be configured for use in a field such as, for example, on-site at a fresh produce farm. Accordingly, the systems and kits can be portable and capable of use in a non-laboratory setting.

In certain embodiments, a portable testing system for detecting the presence or absence of contamination in a field is provided comprising: at least one drop dispensing device 100 and a portable instrument and/or kits for performing an isothermal amplification, such as LAMP, at the point of need (PON).

A number of commercial portable instruments to perform isothermal amplification at the PON are available. Examples of these devices include, without limitation, the 3M Molecular Detection System (3M, St. Paul, MN) for the detection of food-borne pathogens such as Salmonella spp. and Listeria monocytogenes, HumaLoop T and HumaLoop M (HUMAN Diagnostics Worldwide, Wiesbaden, Germany) for detection of Mycobacterium tuberculosis complex, Plasmodium spp., Genie II (OptiGene Ltd., West Sussex, UK) for detection of plant pathogens, and CapitalBio RTisochip (CapitalBio Technology, Beijing, China) for applications such as food safety testing, clinical diagnosis, and the like. The majority of the available tools are challenging to use in the field by non-specialist personnel and require multiple handling of the reagents using pipettors. If the PON is fast and straightforward to use, it can be advantageous to industrial stakeholders for decision-making during pre-season or pre-harvest of fresh produce.

In certain embodiments, the portable instrument to perform isothermal amplification at the PON comprises at least one assay device 200. In certain embodiments, the system is configured to run off a portable power bank such that it can easily be operated from the back of a vehicle or otherwise in the field.

In certain embodiments, a testing system for detecting the presence or absence of contamination in a field comprises at least one drop dispensing device 100, at least one assay device 200, and a heating unit. In certain embodiments, the testing system can further comprise a temperature control system. Additionally or alternatively, the testing system can further comprise an imaging unit.

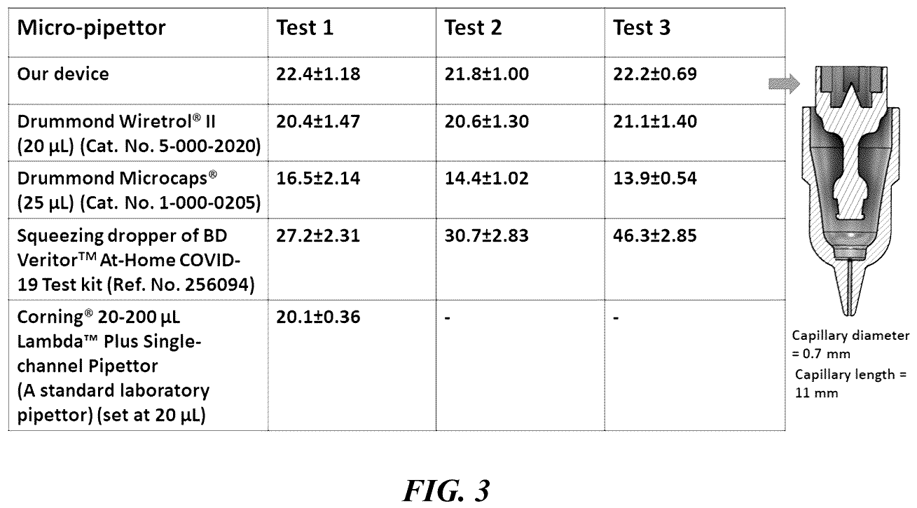

The at least one drop dispensing device 100 can be any of the devices 100 described herein. As compared with the evaluated commercial micro-pipettors (see Examples), systems comprising the drop dispensing device 100 can provide higher precision in dispensing a fixed drop volume (FIG. 3). The majority of the available commercial micro-pipettors use sharp glass capillary tubes or sharp tips that can be hazardous for on-farm applications by non-specialist users. Also, because both the interior and exterior surfaces of the glass capillary tubes are hydrophilic, there is always a chance of the drop remaining stuck to the exhaust tip of the capillary tube; reducing the dispensing precision. On the contrary, certain embodiments of the drop dispensing devices 100 comprise specific surface chemistries to create a hydrophilic interior surface of the capillary tube and a hydrophobic exterior surface of the tip, which can enhance dispensing precision as compared to conventional pipettes. Further, the drop dispensing devices 100 hereof utilize a positive displacement dispensing mechanism that allows for good control of the sample volume.

In certain embodiments, the tip 24 of the drop dispensing device 100 defines a first angle θ along a length of the tip at or between 0-3 degrees, and/or a second angle α of at or between 0-20 degrees at a distal end of the tip; and comprises an outer tip surface. The inner surface of the capillary tube can be hydrophilic, and the outer tip surface is hydrophobic, for example. Additionally or alternatively, an outer surface of the plunger 14 can be hydrophobic.

The assay device 200 can be any of the isothermal amplification assay devices 200 described herein or other isothermal amplification assays known in the art. The heating unit can be coupled with a temperature control system and/or an imaging unit. In certain embodiments, the isothermal amplification assay device comprises two or more paper-based pads 202 positioned in a stacked configuration relative to each other, wherein at least one of the paper-based pads 202 is loaded with one or more reagents 250 (the “reaction pad 202”). The reagent(s) 250 can comprise primer sets for the amplification of a genetic target associated with contamination. The primer sets can be, for example, encoded by at least SEQ ID NOS: 7-27, wherein the genetic target is specific to Bacteroidales. The primer sets can be, for example, encoded by at least SEQ ID NOS: 1-6, wherein the genetic target is specific to E. coli. Additionally, at least one of the paper-based pads 202 does not have any reagents 250 thereon (the “control pad 202”).

As noted above, the system can comprise a heating unit. The heating unit is for applying heat to initiate amplification of the genetic target associated with contamination when the reagents 250 and the sample are combined, for example, on the reaction pad 202 of the assay device 200. In certain embodiments, the heating element is or comprises a water bath or an incubator. The incubator can be any incubator now known or hereinafter developed that would be suitable for field-based testing.

The heating unit can comprise at least one heating element coupled, or otherwise capable of heat transfer, with a tank (e.g., for holding and heating, using the heating element(s), a fluid therein). The at least one heating element can comprise any suitable heating element. In certain embodiments, the heating element comprises a cartridge heater, an immersion heater, a water bath heater element, a ceramic heater, a strip heater, a thermostatic and/or electric heater, a hot plate, or a Bunsen burner. In certain embodiments, the heating element of the system is an 80W, 120V immersion cartridge heater. In certain embodiments, the heating element is a PTC heater. In certain embodiments, the h heating element eater can hold a fixed temperature, for example at or between about 60° C. to about 70° C. (such as at or about 60° C. to 70° C., at or 60° C. to about 70° C., or at or 60° C. to 70° C.). In certain embodiments, the heating element can hold a set temperature, for example at or between about 61° C. to about 69° C. (such as at or about 61° C. to 69° C., at or 61° C. to about 69° C., or at or 61° C. to 69° C.). In certain embodiments, the heating element can hold a set temperature, for example at or between about 62° C. to about 68° C. (such as at or about 62° C. to 68° C., at or 62° C. to about 68° C., or at or 62° C. to 68° C.). In certain embodiments, the heating element can hold a set temperature, for example at or between about 63° C. to about 67° C. (such as at or about 63° C. to 67° C., at or 63° C. to about 67° C., or at or 63° C. to 67° C.). In certain embodiments, the heating element can hold a set temperature, for example at or between about 64° C. to about 66° C. (such as at or about 64° C. to 66° C., at or 64° C. to about 66° C., or at or 64° C. to 66° C.). In certain embodiments, the heating element can hold a set temperature at or about 65° C. (such as at 65° C.).

The heating unit can further comprise one or more submersible pumps to circulate fluid (e.g., water) contained within the tank and facilitate temperature uniformity throughout the fluid.

In certain embodiments, the heating unit comprises a transparent observation window (e.g., formed of a transparent or semi-transparent acrylic sheet or the like) such that results and/or reaction pad 202 of the assay 200 can be visually observed when the assay device 200 is positioned within and/or heated by the heating unit.

The heating unit can further comprise a temperature sensor such as a digital thermometer or the like. The temperature sensor can be positioned on the unit in a location suitable to monitor the temperature of the at least one heating element and/or fluid being heated. For example, the temperature sensor can monitor the temperature of a water bath or interior of an incubator when in use. The temperature sensor can be any suitable temperature sensor known in the art. For example, the temperature sensor can be a waterproof digital temperature sensor. In certain embodiments, the temperature sensor is in electrical communication with and, optionally, controlled by, the temperature control system.

The temperature control system can be any suitable temperature control system. The temperature control system can be a Proportional, Integral, and Derivative (PID) temperature controller or regulating system as is known in the art. In certain embodiments, the temperature control system further comprises a microprocessor and utilizes at least one algorithm to calculate the difference between the desired temperature setpoint and current process temperature and predict how much power to use in subsequent process cycles to ensure the process temperature remains as close as possible to the setpoint irrespective of environmental changes. The microprocessor can comprise a Raspberry Pi 4B. The temperature control system can be in electrical communication with the temperature sensor of the at least one heating unit and, via operation of a PID control algorithm, control operation of (or adjust) the at least one heating unit to achieve a consistent and desired temperature based on the temperature data received from the temperature sensor. In certain embodiments, the temperature control system is integral with (or part of) the heating element or heating unit.

The imaging unit can comprise a fluorescent reader (e.g., a fluorometer), a UV light reader, a camera, or any other device that is suitable to capture and/or analyze colorimetric data and/or changes in a visual result shown in the reaction pads 202 of a assay device 200. The imaging unit can comprise, for example, an autofocus camera mounted adjacent to the heating unit such that the camera can focus on/visualize/capture the reaction pad 202 of a LAMP assay test 200 positioned within the heating unit. The imaging unit can further comprise a platform for housing the imaging components. In certain embodiments, the imaging unit comprises one or more cooling fans positioned to reduce the heat of the camera or other imaging components thereof. In certain embodiments, the imaging unit comprises a high-resolution, autofocus camera. In certain embodiments, the camera of the imaging unit is configured to capture time-lapse images periodically to visualize and track a reaction on the assay device 200.

Within a pre-season period, this system could be used for risk assessment of pathogen contamination from nearby animal operations. For pre-harvest, the testing systems hereof can be used to facilitate determining if the harvested produce is safe to be sent to the market.

Based on Table 1, running the testing systems using the drop dispensing devices 100 and LAMP assay devices 200 hereof can be cost-efficient at a level that is reasonable for future industrial applications. In certain embodiments, more than 50% of the total price of the drop dispensing device 100 corresponds to the resin cost.

TABLE 1
List of materials for colorimetric LAMP using drop dispensers.

| | | | | |
|---|---|---|---|---|
| | | | Amount of use | |
| Item | Source | Unit price | per test | Price per test |

Droplet dispenser

| | | | | | | |
|---|---|---|---|---|---|---|
| High Temp | Formlabs, RS- | $199/1 | L | 10 | mL | $1.99 |
| V2 resin | F2-HTAM-02 | | | | | |
| O-ring | Helipal, Airy- | $2.9/40 | pieces | 2 | pieces | $0.15 |
| | Acc-Oring- | | | | | |
| | 2.5 × 6 mm | | | | | |
| Sub-total | | | | | | $2.14 (55%) |

LAMP assay

| | | | | | | |
|---|---|---|---|---|---|---|
| Magnesium | Sigma-Aldrich, | $68.20/500 | g | 0.0002 | g | $0.00 |
| Sulfate | M2773 | | | | | |
| Potassium | Sigma-Aldrich, | $47.80/500 | g | 0.0004 | g | $0.00 |
| Chloride | P9541 | | | | | |
| Antarctic | New England | $80.00/100 | U | 0.0875 | U | $0.07 |
| Thermolabile | Biolabs, | | | | | |
| UDG | M0372S | | | | | |
| dNTPs | Fisher Scientific, | $634.50/4 | mL | 0.0014 | mL | $0.22 |
| | FERR0182 | | | | | |
| dUTP | Fisher Scientific, | $80.70/250 | μL | 0.0875 | μL | $0.03 |
| | FERR0133 | | | | | |
| Phenol Red | Sigma-Aldrich, | $105.00/25 | g | 0.0094 | g | $0.04 |
| | P3532 | | | | | |
| Betaine | Sigma-Aldrich, | $103.00/7.5 | mL | 0.0001 | mL | $0.00 |
| | B0300-5VL | | | | | |
| EC.stx1 | Life | $68.44/12500 | reactions | 1 | reaction | $0.01 |
| Primer mix | Technologies, | | | | | |
| | N/A | | | | | |
| Warmstart Bst | New England | $296.00 /0.067 | mL | 0.0001 | mL | $0.44 |
| 2.0 DNA | Biolabs, | | | | | |
| Polymerase | M0537M | | | | | |
| Sub-total | | | | | | $0.81 (21%) |

Sample collection

| | | | | | | |
|---|---|---|---|---|---|---|
| BD BBL Dacron | BD, 263000 | $189.5/500 | swabs | 1 | swab | $0.85 |
| Polyester-Tipped | | | | | | |
| Swabs | | | | | | |
| Transparency | Apollo, 617993 | $32.99/100 | sheets | 0.3 | sheet | $0.10 |

| | |
|---|---|
| film | |
| Sub-total | $0.95 (24%) |
| Total | $3.9 |

Kits are also provided for use in connection with on-farm risk assessment of fresh produce. In at least one embodiment, the diagnostic kits comprise: (1) at least one swab for obtaining a sample from a leaf or flag; (2) a molecular diagnostic assay comprising one or more of the isothermal amplification assay devices 200; and (3) one or more drop dispensing devices 100. The drop dispensing devices 100 can be pre-loaded with a solvent within the interior of the liquid holder 16. In certain embodiments, such assays comprise a paper-based biosensor (e.g., assay device 200).

Kits for detecting contamination in a field of interest are also provided. In certain embodiments, such kits comprise: at least one swab for obtaining a sample; at least one drop dispensing device 100; and at least one isothermal amplification assay device 200. The drop dispensing device(s) 100 and the assay device(s) 200 can be any of such devices described herein.

In certain embodiments, the papers (e.g., paper pads 202) of the assay device 200 can be placed inside a cartridge which is coupled with the drop dispenser device 100. The paper pads 202 of the assay device 200 can comprise all needed reagents 250 in a dry state to run the assay. Such combination of the drop dispenser device 100 and the assay device 200 can be a disposable unit. In certain embodiments, the primer sets of the assay device 200 are encoded by eat least SEQ ID NOS: 7-27 and the genetic target is specific to Bacteroidales, and/or SEQ ID NOS: 1-6 and the genetic target is specific to E. coli.

The kits can optionally further comprise a heating element to initiate amplification of the genetic target when the one or more reagents 250 of the assay device 200 and the sample are combined. The heating element can be any described herein in connection with a heating unit of the system.

The kits can further comprise a plurality of collection flags for placement in a field of interest. The collection flags can be or comprise those described in International Patent Application Publication No. WO 2023/225573 or those otherwise described herein. In certain embodiments, the collection flags each comprise a film affixed to a support at a distance away from an end of the support such that, in use, the support can anchor the film a distance above a surface (e.g., the ground) of an area in which the support is positioned (i.e., the field of interest).

In certain embodiments, the kits further comprise a control for comparison with a reacted reaction pad to determine a baseline against which visual results of the reacted reaction pad can be measured. For example, the control can comprise a continuum of colors, each color associated with a concentration of detected contaminant.

The kits hereof can further comprise a vial or other container containing media (e.g., a solvent such as water, a buffer or any other suitable solvent). In at least one exemplary embodiment, the media within the container can be used to pre-wet a leaf or flag prior to swabbing or otherwise collecting a sample therefrom. The container or vial can be sealable.

In operation, the user need only add a sample (e.g., a swabbed sample and/or DNA sample) to the liquid holder 16 of the device 100 and press the plunger 14 to deliver the sample to the paper pads within the cartridge. The cartridge can then be heated using the heating unit of the system to perform the LAMP assay and, using the imaging unit, visualize the results thereof.

Enabling nucleic-acid testing on-farm for pre-season and pre-harvest microbial risk assessments can require tackling several challenges to create an easy-to-use biosensor that can be used by a non-specialist user. The drop dispenser device 100, which can be part of such a user-friendly biosensor, can provide precise and reproducible drops by pushing only one button without adversely affecting the downstream reactions on the cartilage.

### Methods

Methods for identifying a genetic target associated with contamination in a fresh produce sample (and treating the same) are also provided. In at least one embodiment, a method for identification of a pathogen associated with contamination in a sample comprises: providing at least one drop dispensing device 100, providing at least one assay device 200 comprising a reaction pad 202 pre-loaded with a primer set specific to a genetic target associated with contamination in a sample; obtaining a sample from a leaf or flag positioned in a targeted field; resuspending the sample with a solvent housed within the fluid holder 16 of a drop dispensing device 100 (e.g., by inserting the sample into the interior of the fluid holder 16); activating the plunger 14 of the drop dispensing device 100 to deliver at least a drop of the sample/solvent mixture to a reaction pad 202 (pre-loaded with reagents 250) of a assay device 200; heating the assay device 200 to initiate amplification of a genetic target associated with contamination in the sample; and detecting a visual result in the heated assay device 200 indicative of the presence or absence of the targeted pathogen or FIB in the sample. In certain embodiments, if the visual result indicates the presence of at least one pathogen or FIB present in the sample, the method further comprises identifying the type of pathogen or FIB present. In at least one embodiment, if the visual result is indicative of the presence of the targeted pathogen or FIB in the sample, the method further comprises treating the targeted field for the pathogen or FIB, or delaying harvesting the crop from the field.

In certain embodiments, the method for identifying a genetic target associated with contamination of a fresh produce sample comprises: (1) providing at least one drop dispensing device comprising: a liquid holder that defines an interior, the interior in fluid communication with an inlet of the liquid holder and an outlet of the liquid holder, and a plunger configured to be movable up and down within at least a portion of the interior of the liquid holder such that downward movement of the plunger within the interior causes any fluid contained in the interior to be displaced through the outlet of the liquid holder, wherein the outlet of the liquid holder further comprises tip comprising a capillary tube that defines an inner surface in fluid communication with the interior; (2) providing at least one isothermal amplification assay device comprising two or more paper-based pads positioned in a stacked configuration relative to each other, at least one of the paper-based pads comprising a reaction pad loaded with one or more reagents comprising primer sets for the amplification of a genetic target associated with contamination, and at least one of the paper-based pads comprising a control pad that does not have amplification reagents thereon; (3) suspending a sample from a targeted field with a solvent housed within the liquid holder of the drop dispensing device; (4) loading the reaction pad of the assay device with the suspended sample by pressing the plunger of the drop dispensing device down to deliver at least a drop of the combined sample and solvent mixture to the reaction pad; (5) heating the loaded reaction pad of the assay device to initiate amplification of the genetic target if present within the sample; and (6) detecting a visual result in the heated reaction pad indicative of the presence or absence of the contamination in the sample.

The drop can comprise a volume of between about 22 μL-about 35 μL. In certain embodiments, the drop volume comprises a volume of between about 23 μL-about 34 μL. In certain embodiments, the drop volume comprises a volume of between about 24 μL-about 33 μL. In certain embodiments, the drop volume comprises a volume of between about 25 μL-about 32 μL. In certain embodiments, the drop volume comprises a volume of between about 26 μL-about 31 μL. In certain embodiments, the drop volume comprises a volume of between about 27 μL-about 30 μL. In certain embodiments, the drop volume comprises a volume of between about 28 μL-about 29 μL. In certain embodiments, the drop volume comprises a volume of at or about 27 μL (such as 27 μL). All ranges set forth in this paragraph are inclusive of the stated endpoints and encompass all 0.5 μL encompassed by the stated ranges.

At least one primer set of the reagents 250 on the reaction pad 202 can be any of the primer sets described herein. In at least one embodiment, for example, the at least one primer set can be one or more primer sets encoded by SEQ ID NOS: 1-6 and/or SEQ ID NOS: 7-27. In at least one embodiment, the reagents 250 comprise one or more primer sets described in International Patent Application Publication No. WO 2023/225573.

The heating step can be performed between for about 45 to about 120 minutes (such as between about 45 minutes to 120 minutes, 45 minutes to about 120 minutes, or 45-120 minutes). The heating step can be performed for at or between about 60 to about 105 minutes (such as between about 60 minutes to 105 minutes, 60 minutes to about 105 minutes, or 60-105 minutes). The heating step can be performed for at or between about 75 to about 80 minutes (such as between about 75 minutes to 80 minutes, 75 minutes to about 80 minutes, or 75-80 minutes). The heating step can be performed for at or about 60 minutes. Additionally or alternatively, the loaded reaction pad can be heated to a temperature of at or between about 60-70° C. (such as at or between about 60° C. to 70° C., 60° C. to about 70° C., or 60° C. to 70° C.). Additionally or alternatively, the loaded reaction pad can be heated to a temperature of at or between about 61-69° C. (such as at or between about 61° C. to 69° C., 61° C. to about 69° C., or 61° C. to 69° C.). Additionally or alternatively, the loaded reaction pad can be heated to a temperature of at or between about 62-68° C. (such as at or between about 62° C. to 68° C., 62° C. to about 68° C., or 62° C. to 68° C.). Additionally or alternatively, the loaded reaction pad can be heated to a temperature of at or between about 63-67° C. (such as at or between about 63° C. to 67° C., 63° C. to about 67° C., or 63° C. to 67° C.). Additionally or alternatively, the loaded reaction pad can be heated to a temperature of at or between about 64-66° C. (such as at or between about 64° C. to 66° C., 64° C. to about 66° C., or 64° C. to 66° C.). Additionally or alternatively, the loaded reaction pad can be heated to a temperature of at or about 65° C. (such as at or about 65° C.).

In certain embodiments, the heating step is performed by an integrated heating and imaging unit and the method further comprises capturing at least one image of the reaction pad of the assay device.

In at least one illustrative embodiment, the sample comprises a swab sample taken from a leaf or fruit of produce growing in a field or from a flag positioned in a field of interest; however, any sample capable of providing a medium sufficient to detect the targeted pathogen(s) or FIB(s) using the methods hereof can be used. The collection flags can be or comprise those described in International Patent Application Publication No. WO 2023/225573.

In certain embodiments, collection flags for the collection of bioaerosol samples can be used. A collection flag can comprise a film affixed to a support at a distance away from an end of the support such that, in use, the support can anchor the film a distance above a surface of an area in which the support is positioned.

Collection flags can be positioned at various locations on the area of land and allowed to collect bioaerosol contaminates thereon over a period of time. The flags can be positioned any distance apart as desired. In certain embodiments, the flags are positioned at about 10-200 meters intervals in the field of interest. In certain embodiments, the flags are positioned at about 20-180 meters intervals in the field of interest. In certain embodiments, the flags are positioned at about 40-160 meters intervals in the field of interest. In certain embodiments, the flags are positioned at about 60-140 meters intervals in the field of interest. In certain embodiments, the flags are positioned at about 80-120 meters intervals in the field of interest. In certain embodiments, the flags are positioned at about 100-meter intervals in the field of interest.

Any number of collection flags can be employed. In certain embodiments, about 60 collection flags are used per acre in the field of interest. In certain embodiments, about 20-200 collection flags are used per acre in the field of interest. In certain embodiments, about 30-190 collection flags are used per acre in the field of interest. In certain embodiments, about 40-180 collection flags are used per acre in the field of interest. In certain embodiments, about 60-160 collection flags are used per acre in the field of interest. In certain embodiments, about 80-140 collection flags are used per acre in the field of interest. In certain embodiments, about 100-120 collection flags are used per acre in the field of interest. It will be appreciated that any number of flags can be used as desired in a particular setting.

Samples can be collected directly from the film of each of the collection flags after a desired period of time has elapsed.

The contamination can comprise a pathogen or an FIB. The FIB can be E. coli, E. faecalis, or Bacteroidales.

Detecting the visual result produced by the method can be performed using any of the modalities described above (e.g., the imaging unit of the system). In certain embodiments, the visual results can be seen with the naked eye (without the use of additional instruments). In other embodiments, the assay device 200 further comprises one or more indicators associated with each set of loop primers such that detection of a particular indicator is indicative of the associated contamination being present within the sample. Accordingly, the methods can additionally comprise using a fluorescent reader (e.g., a fluorometer), an ultraviolet light reader, or camera to analyze colorimetric data in the visual result.

The method can further comprise quantifying a concentration of the contamination present using the methodologies described herein and/or the imaging unit.

In certain embodiments, if the presence of contamination in the sample is indicated, the method further comprises performing an intervention. The intervention can be crop dependent and/or dependent upon the point of growth. In certain embodiments, an intervention can comprise destroying the fresh produce crop. In certain embodiments, an intervention can comprise additional washing of the fresh produce crop (e.g., post-harvest). In certain embodiments, an intervention can comprise delaying harvest of the fresh produce crop to ameliorate or eliminate the contamination. In certain embodiments, an intervention can comprise not planting a fresh produce crop in a particular field having contamination.

These methods are advantageous over conventional methods because accurate results are more quickly produced, and the methods hereof can be performed on-site in the field (i.e., they do not require a laboratory). In certain embodiments, the methods hereof can provide a visual result indicative of the presence or absence of the genetic target within 45-120 minutes of initiating the heating step (e.g., initiating reaction of the primers with the sample). Further, such reactions can be conducted between about 60-70° C., and optionally at or about 65° C., which is well outside of ambient field temperatures. In certain embodiments, such reactions can be conducted at a temperature of at or between about 61-69° C. (such as at or between about 61° C. to 69° C., 61° C. to about 69° C., or 61° C. to 69° C.). In certain embodiments, such reactions can be conducted at a temperature of at or between about 62-68° C. (such as at or between about 62° C. to 68° C., 62° C. to about 68° C., or 62° C. to 68° C.). In certain embodiments, such reactions can be conducted at a temperature of at or between about 63-67° C. (such as at or between about 63° C. to 67° C., 63° C. to about 67° C., or 63° C. to 67° C.). In certain embodiments, such reactions can be conducted at a temperature of at or between about 64-66° C. (such as at or between about 64° C. to 66° C., 64° C. to about 66° C., or 64° C. to 66° C.). In certain embodiments, such reactions can be conducted at a temperature of at or about 65° C. (such as at or about 65° C.).

In certain embodiments of the method, quantifying the presence or absence of a genetic target associated with contamination in each sample further comprises: providing at least one drop dispensing device 100; providing at least one isothermal amplification assay device 200; suspending a collected sample in a solvent housed within a liquid holder 16 of the drop dispensing device 100; loading a reaction pad 202 of the assay device 200 with the suspended sample by pressing the plunger 14 of the drop dispensing device 100 down to deliver at least a drop of the combined sample and solvent mixture to the reaction pad 202; heating the loaded reaction pad 202 of the assay device 200 to initiate amplification of the genetic target if present within the sample; and detecting a visual result in the heated reaction pad 202 indicative of the presence or absence of the contamination in the sample.

The drop dispensing device 100 and/or the assay device 200 can be any described herein. In certain embodiments, the drop dispensing device 100 comprises a liquid holder 16 that defines an interior, the interior in fluid communication with an inlet 12 of the liquid holder 16 and an outlet 20 of the liquid holder 16, and a plunger 14 configured to be movable up and down within at least a portion of the interior of the liquid holder 16 such that downward movement of the plunger 14 within the interior causes any fluid contained in the interior to be displaced through the outlet 20 of the liquid holder 16. The outlet 20 of the liquid holder 16 can further comprises a tip 24 comprising a capillary tube 22 that defines an inner surface in fluid communication with the interior.

In certain embodiments, the assay device 200 comprises two or more paper-based pads 202 positioned in a stacked configuration relative to each other, at least one of the paper-based pads 202 comprising a reaction pad 202 loaded with one or more reagents 250 comprising primer sets for the amplification of a genetic target associated with contamination, and at least one of the paper-based pads 202 comprising a control pad that does not have amplification reagents thereon. The primer sets of the assay device 200 can be encoded by at least: SEQ ID NOS: 7-27 and the genetic target is specific to Bacteroidales, and/or SEQ ID NOS: 1-6 and the genetic target is specific to E. coli.

In certain embodiments, the heating step is performed for about 45 to about 120 minutes (such as between about 45 minutes to 120 minutes, 45 minutes to about 120 minutes, or 45-120 minutes). The heating step can be performed for at or between about 60 to about 105 minutes (such as between about 60 minutes to 105 minutes, 60 minutes to about 105 minutes, or 60-105 minutes). The heating step can be performed for at or between about 75 to about 80 minutes (such as between about 75 minutes to 80 minutes, 75 minutes to about 80 minutes, or 75-80 minutes). The heating step can be performed for at or about 60 minutes.

Additionally or alternatively, the loaded reaction pad can be heated to a temperature of at or between about 60-70° C. (such as at or between about 60° C. to 70° C., 60° C. to about 70° C., or 60° C. to 70° C.). Additionally or alternatively, the loaded reaction pad can be heated to a temperature of at or between about 61-69° C. (such as at or between about 61° C. to 69° C., 61° C. to about 69° C., or 61° C. to 69° C.). Additionally or alternatively, the loaded reaction pad can be heated to a temperature of at or between about 62-68° C. (such as at or between about 62° C. to 68° C., 62° C. to about 68° C., or 62° C. to 68° C.). Additionally or alternatively, the loaded reaction pad can be heated to a temperature of at or between about 63-67° C. (such as at or between about 63° C. to 67° C., 63° C. to about 67° C., or 63° C. to 67° C.). Additionally or alternatively, the loaded reaction pad can be heated to a temperature of at or between about 64-66° C. (such as at or between about 64° C. to 66° C., 64° C. to about 66° C., or 64° C. to 66° C.). Additionally or alternatively, the loaded reaction pad can be heated to a temperature of at or about 65° C. (such as at or about 65° C.).

Methods for evaluating microbial contamination in an area of land are also provided. This can be especially advantageous if, for example, the area of land is in proximity to, or close proximity to, or adjacent to an animal operation. As used in this context, “proximity” means within one mile from the animal operations (closest border to closest border measurement), “close proximity” means within a 1000 feet distance from the animal operation, and “adjacent” means the animal operation shares one or more property line with the produce operation or test area. The method can also be advantageous to use when the area of land is pre-plant or pre-harvest such that, if contamination is detected, interventions can be performed.

The method for evaluating microbial contamination can employ any of the devices 100, 200 and/or systems described herein. In certain embodiments, a method for evaluating microbial contamination in an area of land comprises: obtaining a plurality of samples from various locations in an area of land of interest (e.g., a fresh produce field); detecting the presence or absence of a genetic target associated with contamination in each sample and, if the genetic target is present, quantifying a concentration of the contamination present in the sample; generating a heat map of the area of land from the detection and quantification data; collecting environmental data from the area of land over a period of time; and integrating the collected environmental data with the heat map to evaluate site-specific contamination risk in the area of land. Integrating molecular identification of contaminates with collected environmental data (e.g., data collected from weather stations) can rapidly provide site-specific risk assessments with respect to pathogens and/or FIBs. This can be especially advantageous when the field at issue is in proximity to an animal operation.

In certain embodiments, the contamination comprises a FIB. The FIB can be any FIB. In certain embodiments, the FIB is Bacteroidales, E. coli, or E. faecalis. As described in detail herein, FIB can be a good indicator of fecal contamination and, due to their obligate anaerobicity (preventing their growth and multiplication in the ambient environment), low natural abundance from non-fecal sources, and high host-specificity (various sequences of the 16S rRNA gene have been designed to detect fecal pollution from specific hosts), can be a valuable indicator of contamination in a fresh produce crop or field.

Obtaining a plurality of samples from various locations on an area of land can be performed using the swabbing techniques described above. In certain embodiments, collection flags for the collection of bioaerosol samples can be used. A collection flag can comprise a film affixed to a support at a distance away from an end of the support such that, in use, the support can anchor the film a distance above a surface of an area in which the support is positioned.

Collection flags can be positioned at various locations on the area of land and allowed to collect bioaerosol contaminates thereon over a period of time. The flags can be positioned any distance apart as desired. In certain embodiments, the flags are positioned at about 10-200 meters intervals on the targeted land. In certain embodiments, the flags are positioned at about 20-180 meters intervals on the targeted land. In certain embodiments, the flags are positioned at about 40-160 meters intervals on the targeted land. In certain embodiments, the flags are positioned at about 60-140 meters intervals on the targeted land. In certain embodiments, the flags are positioned at about 80-120 meters intervals on the targeted land. In certain embodiments, the flags are positioned at about 100-meter intervals on the targeted land.

Any number of collection flags can be employed. In certain embodiments, about 60 collection flags are used per acre of targeted land. In certain embodiments, about 1-200 collection flags are used per acre of targeted land. In certain embodiments, about 10-190 collection flags are used per acre of targeted land. In certain embodiments, about 20-180 collection flags are used per acre of targeted land. In certain embodiments, about 30-160 collection flags are used per acre of targeted land. In certain embodiments, about 40-140 collection flags are used per acre of targeted land. In certain embodiments, about 50-120 collection flags are used per acre of targeted land. In certain embodiments, about 60-100 collection flags are used per acre of targeted land. In certain embodiments, about 70-80 collection flags are used per acre of targeted land. It will be appreciated that any number of flags can be used as desired in a particular setting.

Samples can be collected directly from the film of each of the collection flags after a desired period of time has elapsed.

The heat map can comprise a plurality of cells, each of the plurality of cells corresponding to an area in the area of land and associated with a value indicative of a concentration of the contamination in the corresponding area. The step of generating the heat map can be performed by transforming the highest concentrations of contamination detected in a given geographic area (e.g., the field of interest) into red values and displaying the lowest concentration values in white values, and values in between represented as a proportional spectrum between the two colors. Different colors can be used for high and low values as desired and is known in the art. The relative values and colors can be consistently applied across the full time period measured (e.g., a day or week, for example) so that a single concentration, for example, is the same color across the full time range.

In certain embodiments, the heat map can include an aerial/satellite image of the given geographic area (e.g., the field of interest) with an overlay of colored regions, where different colors of the colored regions correspond to different value ranges of a given parameter (e.g., as described). In certain embodiments, the heat map can be generated through the use of interpolation algorithms (e.g., as described herein or as otherwise known in the art) to facilitate generation of the heat maps for a given geographic area based on a given input data set for the given geographic area.

The heat map can be combined in a mashup so that changes in environmental data collected can be compared with contamination concentration as the plots are aligned in the field of interest. In certain embodiments, the environmental data collected is overlaid with the contamination concentration in the field of interest to evaluate environmental effects on different contamination sources (e.g., a proximally located animal operation). In certain embodiments, this allows for contamination information to be evaluated across different temperatures, wind speeds and directions, etc.

The environmental data can comprise weather data collected from one or more weather stations positioned on the area of land to collect such data. In certain embodiments, the environmental data comprises temperature data, pressure data, humidity data, daily rain data, ultraviolet (UV) and solar radiation data, wind speed data, wind direction data, air quality data, or any combination of two or more of the foregoing. The weather stations can be any weather stations known in the art. The weather station can comprise an all-in-one weather station. The weather station can be solar powered, or battery powered. In certain embodiments, the weather station can measure at least temperature, rainfall, relative humidity, and wind direction. The weather station can be in wired or wireless communication (e.g., via the Internet or Bluetooth) with a computer.

As noted above, the method comprises detecting the presence or absence of a genetic target associated with contamination in the sample. This detection step can be performed using a PCR assay or an isothermal amplification assay, for example. In certain embodiments, this detection step is performed using any of the assay devices 200 described herein. In certain embodiments, the detection step is performed using a combination of any of the drop dispensing devices 100 and any of the assay devices 200 described herein. It will be appreciated that both PCR and isothermal amplification assays can also quantify the concentration of a contamination identified. The primer sets of the assay device 200 can be, for example, encoded by at least SEQ ID NOS: 7-27 and the genetic target is specific to Bacteroidales, and/or SEQ ID NOS: 1-6 and the genetic target is specific to Escherichia coli.

The method hereof can be performed by the systems described herein. For example, and without limitation, the heating step can be performed using the heating unit of the system.

In certain embodiments, the heating step of the method for evaluating microbial contamination can be performed for about 45 to about 120 minutes (inclusive of the stated endpoints and all 1-minute increments encompassed by the stated range). In certain embodiments, the loaded reaction pad is heated for 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, or 120 minutes.

In certain embodiments, the loaded reaction pad is heated to a temperature of at or between 60-70° C. to facilitate the isothermal reaction. In certain embodiments, the loaded reaction pad is heated to a temperature of 65° C. to facilitate the isothermal reaction.

The method can further comprise capturing at least one image of the heated reaction pad of the assay device (e.g., the assay device 200) and quantifying the concentration of the contamination in the sample from the image. This step can be performed using the imaging unit of the system or any other camera or imaging device. Quantifying the concentration of the contamination from the at least one image can, for example, utilizing one of more algorithms known in the art or otherwise provided herein. In certain embodiments, the quantifying step is performed by the naked eye.

The area of land can be in proximity to, or close proximity to, an animal operation. The animal operation can be a swine operation. The animal operation can be a cattle operation or feedlot, the animal operation can be a chicken operation. The animal operation can be any mammalian operation comprising numerous mammalian animals.

The method can be performed at different stages of growth on the area of land. For example, the method can be performed pre-plant (e.g., before the area is seeded or otherwise planted). The method can be performed pre-harvest (e.g., before a fresh produce crop is harvested). In certain embodiments, the method is performed at any stage pre-plant or of growth of a fresh produce crop.

If contamination is detected, the method can further comprise identifying a source of the contamination from the environmental data. Identifying the source of contamination from the environmental data can comprise assessing the environmental data for indications of its origin and method of transmission to the area of interest (e.g., the wind, waterflow, etc.). For example, wind data displayed in conjunction with the heat map can indicate that contamination from an adjacent feedlot consistently transmits bioaerosol contaminates into a certain area of a field of interest.

Identifying site-specific risk of contamination can be useful in determining what to plant, when to harvest a crop, and if any interventions may be required or beneficial.

Methods for generating a heat map for a crop-field of interest are also provided. In heat map technologies, values are determined by using continuous or discrete color levels. In this way, distribution and a change rule of a variable in two-dimensional space can be demonstrated vividly and naturally. Therefore, a heat map can be applied to various scenarios to present density data and reflect a region feature. For example, when density data of different regions is presented by using color levels in a heat map in an electronic map, a “hot region” in which data is dense and a “cold region” in which data is sparse can be clearly presented on the map to demonstrate important statistics such as contamination distribution.

In certain embodiments, the method of generating a heat map comprises: obtaining a plurality of samples from various locations in a field of interest; detecting the presence or absence of a genetic target associated with contamination in each sample and, if the genetic target is present, quantifying a concentration of the contamination present in the sample; collecting environmental data from the field of interest over a period of time; and integrating the collected environmental data and the detection and quantification data to generate a heat map showing contamination in the field of interest. The contamination density data of different regions in the heat map can be presented using color levels. In certain embodiments, the contamination is FIB. In certain embodiments, the contamination is Bacteroidales. In certain embodiments, if contamination is detected, the method further comprises identifying a source of the contamination from the environmental data.

### General

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise.

When ranges are used herein, all combinations and sub-combinations of ranges and specific embodiments therein are intended to be included.

Additionally, the term “about” or “approximately” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Additionally, in describing representative embodiments, a method and/or process may have been presented as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass all modifications and changes apparent to those of ordinary skill in the art based on this disclosure.

### EXAMPLES

The following examples illustrate certain specific embodiments of the present disclosure and are not meant to limit the scope of the claimed invention in any way.

### Example 1

### Design and Fabrication of the Drop Dispenser Device

All computer-aided designs were performed using Fusion 360 software (Autodesk, Inc., San Fransisco, CA). The device consisted of two main parts: a plunger that moves up and down when operated by the user, and a liquid holder with a capillary tube at the discharge (FIG. 1A). After designing the device in Fusion 360, the Standard Triangle Language (.stl) files were exported and transferred to a Form 3B stereolithography 3D printer (Formlabs, Somerville, MA) to fabricate the 3D-printed devices.

Different resin types were used and tested during 3D printing including High Temp V2 (Formlabs, Somerville, MA; RS-F2-HTAM-02), Rigid 4000 V1 (Formlabs, Somerville, MA; RS-F2-RGWH-01), BioMed Clear V1 (Formlabs, Somerville, MA; RS-F2-BMCL-01), or Clear V4 resins (Formlabs, Somerville, MA; RS-F2-GPCL-04).

To develop devices of different drop volumes, various design parameters were modified. Devices with different tip lengths, capillary tube diameters, discharge cylinder depths, and tip diameters and angles were fabricated using the selected resin and tested. The fabricated devices were then rinsed using isopropyl alcohol (IPA) for 30 minutes and cured by exposure to ultraviolet (UV) light at 55° C. for 30 minutes. FIG. 1B shows two 3D-printed parts as well as two O-rings (Helipal, Airy-Acc-Oring-2.5×6 mm) that were used to provide sealing.

### Example 2

### Surface Treatment of the Drop Dispenser Device

A number of surface treatment approaches to enhance the reliability of the drop dispenser were studied. These approaches included exposure to plasma for the different duration (2 or 4 minutes), and exposure to polyethylene glycol (PEG) 400 (ThermoFisher Scientific International LLC, Waltham, MA; P167-1) for 24 hours after plasma treatment by either dipping the device in the PEG 400 or only pouring 600 μL of PEG 400 into the interior of the device.

To perform the latter approach (FIG. 1C), the tip of the liquid holder was wrapped with multiple layers of Parafilm wrapping film (ThermoFisher Scientific International LLC, Waltham, MA; S37440) before exposing it to plasma and PEG 400. The plasma treatment was performed using an air plasma generator (Plasma Etch, Inc., Carson City, NV; PE-25) at about 0.2 Torr (26.7 Pa). This treatment approach kept the outer tip surface hydrophobic but made the inner capillary tube hydrophilic.

After surface treatment, the assembled drop dispenser-including the plunger, O-rings, and the liquid holder—was thoroughly washed with isopropyl alcohol (IPA) for 10 minutes and rinsed with ultra-pure water to remove any residue that could interfere with LAMP reactions. The clean devices were dried using an air gun and stored in separate re-closable Polypropylene bags (Uline, Pleasant Prairie, WI; S-17954) for future use.

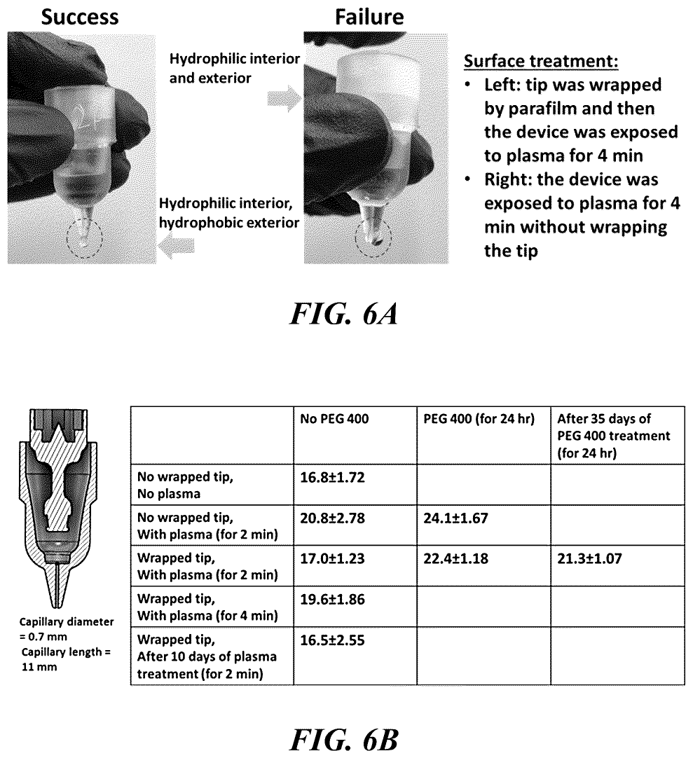

Several drop dispensers were initially fabricated out of High Temp V2 resin and tested to determine the best surface treatment approach. Devices without any surface treatment could not hold the liquid within the liquid holder. Generally, plasma treatment was effective to improve holding the liquid within the device. However, drop generation was only possible for those devices whose tips were already wrapped with a Parafilm wrapping film layer during plasma treatment (FIG. 6A).

Devices treated with a 2-minute plasma exposure better-dispensed drops than those with a 4-minute exposure and provided less variability (FIG. 6B). The 2-minute plasma-treated devices were tested twice with 10-day intervals and it was observed that the precision of the drop dispensers decreased over time (FIG. 6B). Therefore, immediately after plasma treatment, the devices were also exposed to PEG 400. The most satisfactory performance (i.e., less variability) was observed when the PEG 400 was poured within the liquid holder while the tip was wrapped with Parafilm wrapping film (FIG. 6B). Such an approach provided a liquid holder with a hydrophilic capillary tube and a hydrophobic outer tip surface. This feature helped the device to 1) hold the liquid within the capillary tube when the dispenser was not operating, 2) quickly refill the capillary tube after each dispense, and 3) prevent the drop from sticking to the tip outer surface during dispensing. After one week, there was no notable change in the dispensing performance. Therefore, this surface treatment approach was used for additional testing.

### Example 3

### Precision Tests of the Drop Dispenser Device

The efficacy of the drop dispenser device was tested at various stages of development. Devices made from multiple resins (e.g., High Temp V2 (Formlabs, Somerville, MA; RS-F2-HTAM-02), Rigid 4000 V1 (Formlabs, Somerville, MA; RS-F2-RGWH-01), BioMed Clear V1 (Formlabs, Somerville, MA; RS-F2-BMCL-01), or Clear V4 resins (Formlabs, Somerville, MA; RS-F2-GPCL-04)), were initially tested and compared to identify the most appropriate resin that allowed for the highest precision of drop generation using the device.

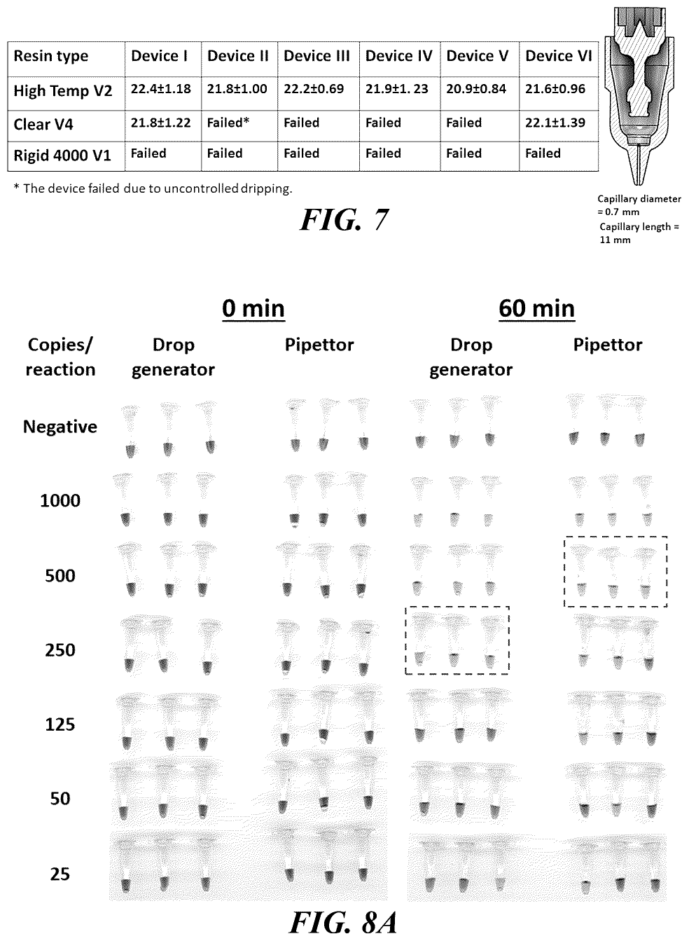

Four to six devices from each of the above-listed resins were fabricated and tested for the generation of 30 drops. A comparison among the results of drop dispensing for all other resins supported that the High Temp V2 resin was the best choice in terms of precision of drop volume and the least number of failed devices (FIG. 7). Generally, plasma and PEG 400 treatments were not effective on devices made from Rigid 4000 V1 resin and all such devices failed due to uncontrolled dripping. Most of the devices made from BioMed Clear V1 resins had blocked capillary tubes after fabrication or surface treatment. Some of the devices made from Clear V4 resin provided similar dispensing performance as the High Temp V2 resin, however, most failed due to uncontrolled dripping (FIG. 7).

Several geometric factors in the device were also evaluated to determine their effects on the drop volumes. These factors were the tip length, tip point diameter, the volume of the dispensing cylinder, the diameter of the capillary tube, and the angle of the capillary tube and the tip point. Among these factors, the angle of the tip (i.e., a in FIG. 2) was the most effective. As shown in FIGS. 2A-2B, changing a from 0 to 15 degrees changed the capacity of the drop dispenser device from 21.5 μL to 32.4 μL with reasonable precision. Increasing a to higher than 15 degrees led to uncontrolled dripping. All the upcoming experiments were performed using devices with α=0 since it helped to keep the LAMP reaction volume at 30 μL.

Next, 6 devices were fabricated from High Temp V2 resin and used for over-time precision tests (α=0; Device I, Device II, Device III, Device IV, Device V, and Device VI). The means of the measured drop volumes were tested for each device after 0, 3, 7, 10, 14, 17, 21, 28, and 35 days from PEG 400 treatment (Table 2). Between the tests, the devices were stored at room temperature in specific boxes. For each test, the device was used to generate 30 drops. The mass of each drop was measured using a Mettler Toledo mass balance (ThermoFisher Scientific International LLC, Waltham, MA; 01-912-402)—with an accuracy of ±0.0001g—and converted to volume units, assuming a water density of 1000 kg/m³. After each test, the device was dried using an air gun and stored until the next experiment.

TABLE 2
Performance of the drop dispensers fabricated from High Temp V2 resin for several days after surface
treatment with plasma and PEG 400.

| Device | | | | | | | | | |
|---|---|---|---|---|---|---|---|---|---|
| No. | Day 0 | Day 3 | Day 7 | Day 10 | Day 14 | Day 17 | Day 21 | Day 28 | Day 35 |
| Device | 22.4 ± | 21.4 ± | 21.2 ± | 21.5 ± | 22.4 ± | 22.2 ± | 21.3 ± | 20.7 ± | 21.3 ± |
| I | 1.18 | 1.22 | 1.14 | 1.21 | 0.80 | 0.88 | 1.40 | 0.82 | 1.07 |
| Device | 21.8 ± | 21.5 ± | 21.6 ± | 21.1 ± | 22.3 ± | 21.3 ± | 22.3 ± | 21.5 ± | Failed^{#} |
| II | 1.00 | 0.96 | 0.85 | 1.30 | 0.89 | 1.17 | 0.91 | 1.08 | |
| Device | 22.2 ± | 22.4 ± | 21.5 ± | 21.0 ± | 21.4 ± | 21.4 ± | 22.2 ± | 22.0 ± | Failed^{#} |
| III | 0.69 | 1.14 | 1.12 | 1.22 | 1.06 | 1.50 | 0.91 | 1.09 | |
| Device | 21.9 ± | 22.0 ± | 21.09 ± | 22.1 ± | 22.5 ± | 22.6 ± | 22.6 ± | 22.2 ± | Failed^{#} |
| IV | 1.23 | 1.12 | 1.04 | 0.97 | 0.85 | 1.08 | 1.16 | 1.14 | |
| Device | 20.9 ± | 20.9 ± | 20.8 ± | 21.3 ± | 22.3 ± | 21.4 ± | 22.3 ± | 22.2 ± | Failed^{#} |
| V | 0.84 | 0.88 | 1.11 | 1.22 | 0.88 | 0.98 | 1.13 | 1.12 | |
| Device | 21.6 ± | 21.4 ± | 22.2 ± | 22.7 ± | 23.0 ± | 22.3 ± | 23.1 ± | 22.0 ± | Failed^{#} |
| VI | 0.96 | 1.08 | 0.96 | 0.89 | 0.76 | 1.03 | 0.76 | 1.07 | |

^{#}Devices failed due to uncontrolled dripping after several usage.

The precision tests were continued until at least one of the devices failed by showing uncontrolled dripping after multiple applications. After 35 days of repeated use and storage at room temperature, some of the devices could no longer keep the liquid inside the liquid holder. Therefore, a shelf life for the devices at room temperature was considered to be 28 days.

To understand how multiple devices and multiple days of repeated usage affect the drop dispensing performance, a two-way analysis of variance (ANOVA) was conducted on the obtained data set. As shown in Table 3, for a significance level of α=0.001, the effects of multiple devices, multiple days of application, and their interactions are significant.

TABLE 3
Results of the two-way ANOVA on the performance data
obtained from multiple testing of the drop dispenser.

| Source of | | | | | |
|---|---|---|---|---|---|
| variation | SS | df | MS | F | P-value |
| Devices | 124.4395 | 5 | 24.88789 | 22.45187 | 1.04E−21 |
| Days of | 113.1684 | 7 | 16.16692 | 16.16692 | 2E−18 |
| application | | | | | |
| Interaction | 256.9596 | 35 | 7.341704 | 6.623098 | 4.14E−28 |
| Within | 1543.032 | 1392 | 1.1085 | | |
| Total | 2037.6 | 1439 | | | |

To further determine for which devices and on what days the performances were significantly different, a Tukey test was conducted to compare the means on various days. The results are shown in Table 4.

TABLE 4
Results of the Tukey test on the performance of the drop dispensers over
several days of application after PEG 400 treatment .*

| Compared | Device | Device | Device | Device | Device | Device |
|---|---|---|---|---|---|---|
| days | I | II | III | IV | V | VI |
| 0-3 | — | — | — | — | — | — |
| 0-7 | * | — | — | — | — | — |
| 0-10 | — | — | * | — | — | — |
| 0-14 | — | — | — | — | * | * |
| 0-17 | — | — | — | — | — | — |
| 0-21 | * | — | — | — | * | * |
| 0-28 | * | — | — | — | * | — |
| 3-7 | — | — | — | — | — | — |
| 3-10 | — | — | * | — | — | * |
| 3-14 | — | — | — | — | * | * |
| 3-17 | — | — | — | — | — | — |
| 3-21 | — | — | — | — | * | * |
| 3-28 | — | — | — | — | * | — |
| 7-10 | — | — | — | — | — | — |
| 7-14 | * | — | — | — | * | — |
| 7-17 | — | — | — | — | — | — |
| 7-21 | — | — | — | — | * | — |
| 7-28 | — | — | — | — | * | — |
| 10-14 | — | * | — | — | — | — |
| 10-17 | — | — | — | — | — | — |
| 10-21 | — | * | * | — | — | — |
| 10-28 | — | — | — | — | — | — |
| 14-17 | — | — | — | — | — | — |
| 14-21 | * | — | — | — | — | — |
| 14-28 | * | — | — | — | — | — |
| 17-21 | — | — | — | — | — | — |
| 17-28 | * | — | — | — | — | — |
| 21-28 | — | — | — | — | — | — |

*The absolute difference of the means were calculated from the ANOVA results shown in Table 3. Critical value = Q *sqrt(MS/n.obs), Q: from Studentized Range q Table, MS: From two-way ANOVA results, n.obs: number of observation for each test = 30. The critical value at alpha = 0.001 was 1.11931935.
— means there is no significant difference;
* means there was a significant difference.

Comparing the data from day 0 with day 28, two devices (Devices I and V) showed significant differences in the drop volumes. For Device IV, no significant differences were observed among all days of usage. Also, two other devices (Devices II and III) only showed significant differences among two or three compared cases. These observations highlight the potential of this design to generate statistically similar drop sizes for future applications. Some of this variability may be due to the use of resins for prototyping. For future fabrication in commercial use, these devices could be made using mold injection that can provide a better surface property, and therefore, a better drop dispensing performance, than resins.

Finally, the precision and reproducibility of drop generation was tested at multiple days for a number of commercial micro-pipettors to compare with the devices hereof. As shown in FIG. 3, the devices hereof provided comparable or better precision and reproducibility in drop generation as compared to several commercial micro-pipettors that are meant for point-of-care applications.

### Example 4

### Bacterial Strains, Culturing, and Quantification

The bacterial strain, Escherichia coli (Migula) Castellani and Chalmers (ATCC® 35150™) was cultured overnight in 3 mL of brain heart infusion (BHI) growth medium (VWR International, LLC, Radnor, PA; 95021-488) using a shaker-incubator at 37° C. To provide a subculture, 3 μL of the culture was again transferred to a 3-mL fresh BHI medium and incubated for another 16 hours. Then, the bacteria were either used for DNA extraction, or the dilutions were used to artificially contaminate the collection flags (in accordance with the methodologies described in Example 8 below).

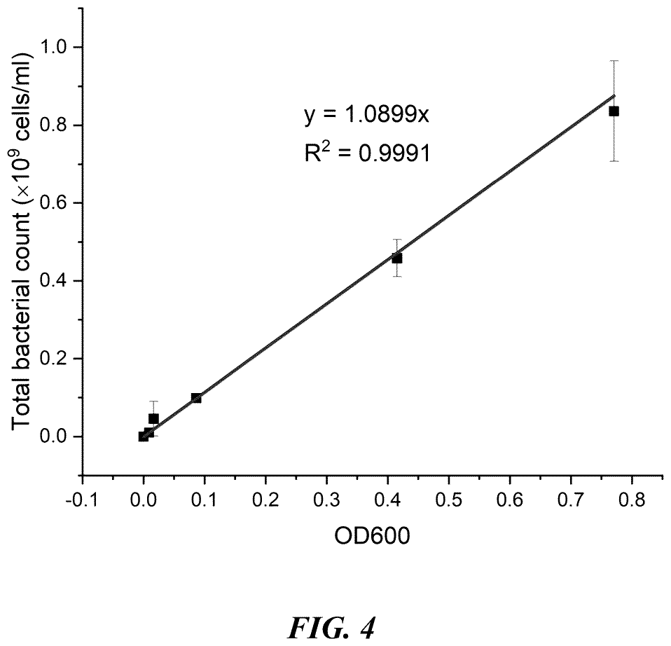

To enable bacterial counting using optical density spectroscopy at 600 nm (OD₆₀₀), a calibration curve was prepared. For this purpose, 1 mL of the initial bacterial culture in the BHI broth was centrifuged at 9000 rpm for 1 minute. After removing the supernatant liquid, the cells were resuspended and well-mixed in the same amount of water and centrifuged again. After a second resuspension in the same amount of water, several serial dilutions with dilution factors of 1, 2, 10, 50, and 100 were prepared in three replicates using molecular biology-grade water. For each dilution, the OD₆₀₀ was read using a CLARIOstar microplate reader (BMG Labtech Inc., Ortenberg, Germany). The same dilutions were immediately used to count the cells using a QUANTOM Tx microbial cell counter (Logos Biosystems, Anyang-si, South Korea), following the manufacturer's protocols for total bacterial counting (QUANTOM Total Cell Staining Kit, Q13501). The calibration curve and corresponding data are shown in FIG. 4.

### Example 5

### DNA Extraction, Purification, and Quantification

DNA extraction was performed using the Invitrogen PureLink Genomic DNA Mini Kit (ThermoFisher Scientific International LLC, Waltham, MA; K182001). 1 mL of overnight bacterial culture was centrifuged to harvest the cell pellet. The cell pellet was re-suspended in 180 μL PureLink Genomic Digestion Buffer. Then, 20 μL of Proteinase K was added to lyse the cells.

After vortexing briefly (for about 1 minute), the tube was incubated at 55° C. to complete cell lysis. A homogeneous mix of the lysate was obtained after adding and vortexing 20 μL RNase A, 200 μL PureLink Genomic Lysis/Binding Buffer, and 200 μL 96-100% ethanol. 640 μL of the lysate was added to a PureLink Spin Column and centrifuged at 10000×g for 1 minute at room temperature. Then the spin column was placed into a clean PureLink collection tube. 500 μL of Wash Buffer 1 was added to the column and the column was centrifuged at 10000×g for 1 minute at room temperature. Then, the spin column was placed into a clean PureLink collection tube. 500 μL of Wash Buffer 2 prepared with ethanol was added to the column and the column was centrifuged for 3 minutes at room temperature.

The DNA was eluted by placing the spin column in a sterile 1.5-mL microcentrifuge tube. Then 30 μL of PureLink Genomic Elution Buffer was added to the column and the column was incubated for 1 minute and centrifuged at maximum speed (14000 rpm) for 1 minute. This elution step was repeated twice, and the DNA was gathered in a 1.5-mL microcentrifuge tube and stored at −20° C. until used for experiments.

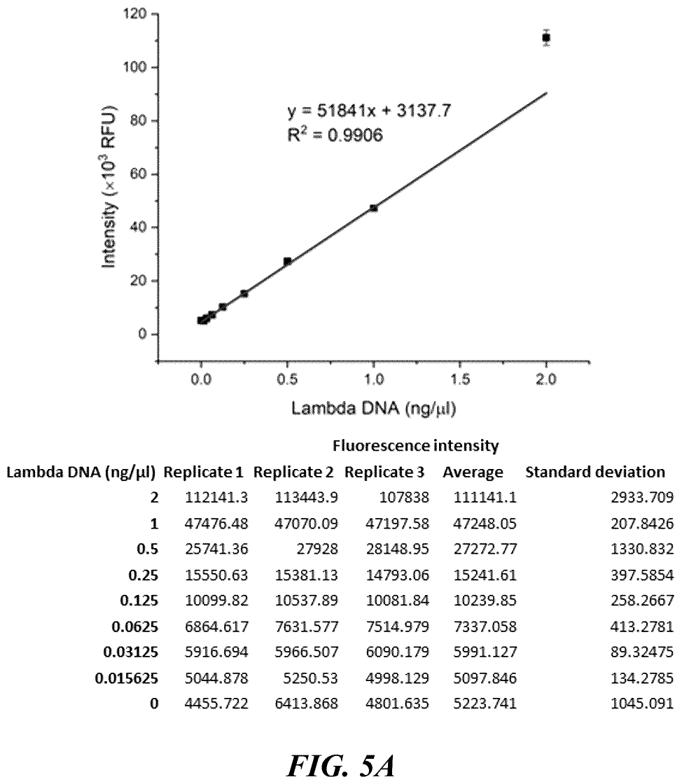

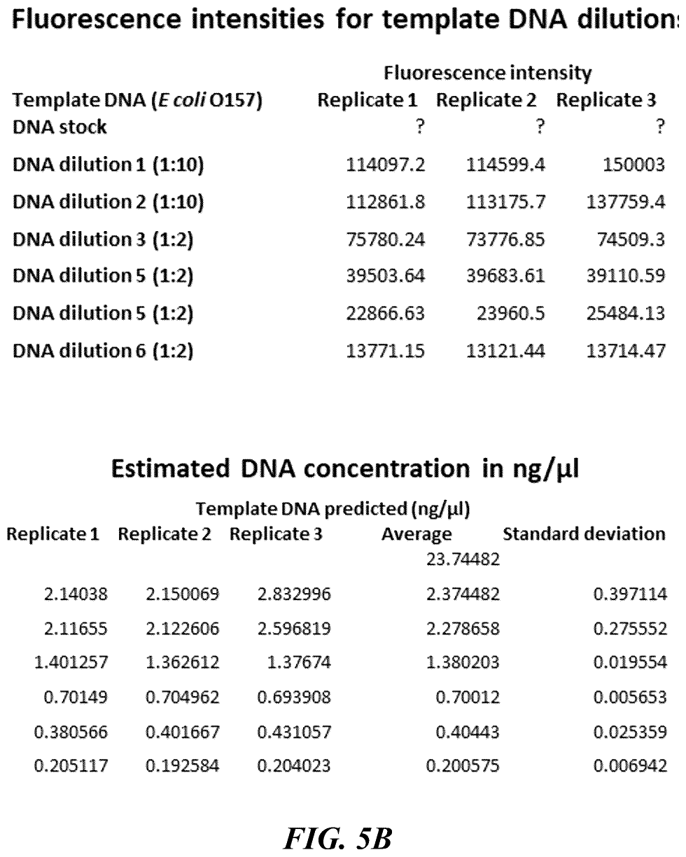

To quantify DNA concentration, 50 L of serial dilutions of synthetic DNA (i.e., Lambda DNA; ThermoFisher Scientific International LLC, Waltham, MA; SD0011) were prepared using Invitrogen 1× TE buffer (ThermoFisher Scientific International LLC, Waltham, MA; 12-090-015). These dilutions are shown in FIG. 5A. Also, using 5 μL of the genomic DNA, 50 μL serial dilutions were prepared (FIG. 5B). 50 μL of diluted Invitrogen PicoGreen dye (Fisher Scientific, P11496), which was a mix of 6.5 μL dye in 1293.5 μL water, was added to all samples and incubated for 5 minutes at room temperature.

Using a 96-well PCR plate, 25 μL of each DNA sample was added to 3 wells. After sealing the plate using a PCR film, the plate was incubated in a thermocycler at 25° C. After 3 thermal “cycles” at a constant temperature, the PicoGreen fluorescent intensity data were retrieved. Using linear regression, a trendline was obtained for the intensity versus the concentration range of the Lambda DNA (FIG. 5A). Then, the equation of the trendline was used to estimate the concentration of the extracted genomic DNA which was 23.7 ng/μL (FIG. 5B).

### Example 6

### LAMP Reaction Mix Preparation

By revising the available protocols for LAMP mix preparation described in Davidson et al., A paper-based colorimetric molecular test for SARS-COV-2 in saliva, Biosensors & Bioelectronics: X 9:100076 (2021) and Wang et al. (2021), supra, a homemade 4× LAMP mix was developed, which consisted of 100 μL KCl (1000 mM; Sigma-Aldrich Corporation, St. Louis, MO; P9541), 160 μL MgSO₄ (100 mM; Sigma-Aldrich Corporation, St. Louis, MO; M2773), 112 μL Deoxynucleotide triphosphate (dNTP) (25 mM; Fisher Scientific International LLC, Waltham MA; FERR0182), 2.8 μL Deoxyuridine Triphosphate (dUTP) (100 mM; Fisher Scientific International LLC, Waltham MA; FERR0133), 0.4 μL Antarctic Thermolabile UDG (1 U/μL; New England Biolabs, Ipswich, MA; M0372S), 5.4 μL Bst2.0 DNA Polymerase (120 U/μL; New England Biolabs, Ipswich, MA; M0537M), 20 μL phenol red solution (25 mM; Sigma-Aldrich Corporation, St. Louis, MO; P3532), and 99.4 μL nuclease-free water (Fisher Scientific International LLC, Waltham MA; 43-879-36).

After mixing, the pH was adjusted to about 7.8-7.9 using potassium hydroxide (KOH) (0.1 M or 1 M) leading to a red, but not pink, solution. The pH measurements were performed by a micro-pH electrode (Fisher Scientific International LLC, Waltham MA; 11-747-328). To prepare the 20×LAMP primer mix, the primer sets shown in Table 5 were mixed by adding 80 μL FIP, 80 μL BIP, 20 μL LF, 20 μL LB, 10 μL F3, 10 μL B3, and 30 μL nuclease-free water. After mixing, the primer mix was heated at 95° C. for 10 minutes prior to usage.

TABLE 5
LAMP primer set used to detect E. coli
O157:H7 by targeting stxI gene

| | | SEQ |
|---|---|---|
| | | ID |
| Primer name | Sequence (5′-3′) | NO. |
| EC.stx1-F3 | TGATTTTTCACATGTTACCTTTC | 1 |
| EC.stx1-B3 | TAACATCGCTCTTGCCAC | 2 |
| EC.stx1-FIP | CCTGCAACACGCTGTAACGTCAGGTACAAC | 3 |
| | AGCGGTTA | |
| EC.stx1-BIP | AGTCGTACGGGGATGCAGATAGTGAGGTTC | 4 |
| | CACTATGC | |
| EC.stx1-LF | GTATAGCTACTGTCACCAGACAATG | 5 |
| EC.stx1-LB | AAATCGCCATTCGTTGACTACT | 6 |

The final LAMP master mix (about 9 μL per reaction) was prepared by using 7.5 μL of the 4× mix, 1.5 μL of 20× primer mix, and 0.12 of betaine (5 M; Sigma-Aldrich Corporation, St. Louis, MO; B0300-5VL). Then, about 21 μL of template-either extracted DNA dilutions (see Example 7) or resuspended bacterial culture (see Example 8)—was added using either an Eppendorf 20-200 μL pipettor or our drop dispenser.

### Example 7

### LAMP Assays on Extracted Bacterial DNA Using Drop Dispenser Device

To investigate the efficacy of the drop dispenser device in performing LAMP assays, as compared with commercial lab-based pipettors, studies were conducted to measure the limit of detection (LoD) in each condition. Six levels of E. coli O157:H7 DNA concentrations were assessed, including 1000, 500, 250, 125, 50, and 25 copies per reaction, as well as negative controls (0 DNA concentration). For each level, three replicates were considered, and the LoD tests were conducted three times on different days to ensure the results were repeatable. Each assay was performed in a 0.2-mL PCR tube. Therefore, a total of 42 reactions per LoD test were performed.

After preparation of all reaction mixes and adding the DNA templates (following the protocol described in Example 6 above), they were placed in 3D-printed tube holders-made from a Rigid 4000 V1 resin (Formlabs, Somerville, MA; RS-F2-RGWH-01) with white color—and submerged in a water bath for 60 minutes at 65° C. The temperature of the water bath had been verified previously using a Hti HT-04 Thermal Imaging Camera (see Pascual-Garrigos et al., On-farm colorimetric detection of Pasteurella multocida, Mannheimia haemolytica, and Histophilus somni in crude bovine nasal samples, Veterinary Research 52 (1): 126 (2021)).

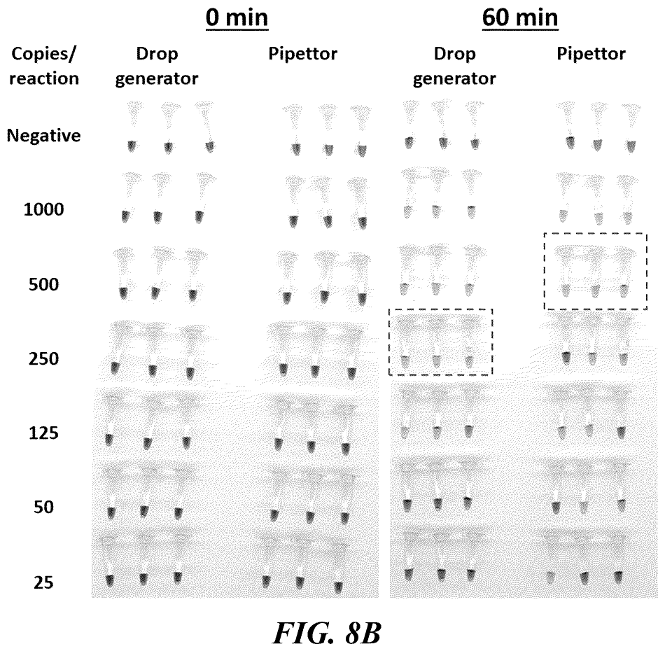

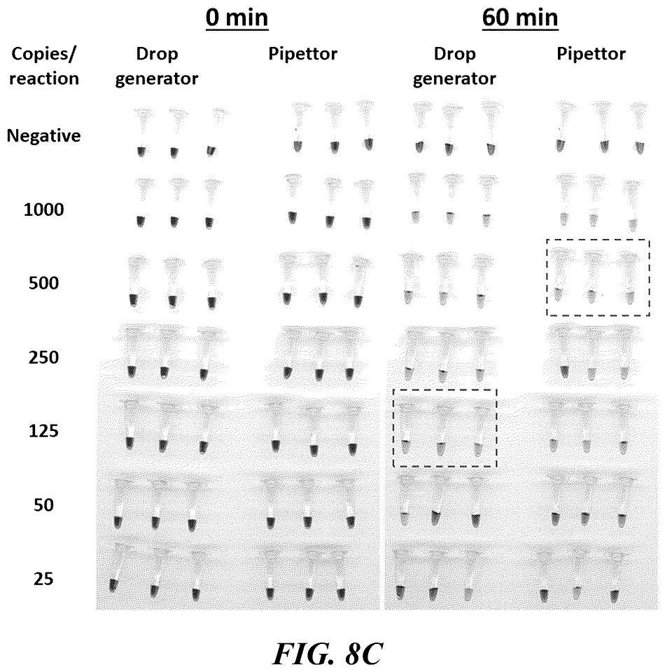

Table 6 and FIGS. 8A-8C show the representative LAMP results when using various concentrations of extracted DNA of E. coli O157:H7 as the template, and the EC.stx1.1 primer set (Table 5).

TABLE 6
Results of the LAMP assays using the drop dispenser vs. a
standard pipettor.

| DNA | | | | | | |
|---|---|---|---|---|---|---|
| concentration | Assay 1 | | Assay 2 | | Assay3 | |
| (copies/reaction) | Device | Pipettor | Device | Pipettor | Device | Pipettor |
| 1000 | +, +, + | +, +, + | +, +, + | +, +, + | +, +, + | +, +, + |
| 500 | +, +, + | +, +, + | +, +, + | +, +, + | +, +, + | +, +, + |
| 250 | +, +, + | +, +, − | +, +, + | −, +, + | +, +, + | −, +++ |
| 125 | −, −, − | +, −, − | +, +, − | +, +, − | +, +, + | +, +, + |
| 50 | −, −, − | −, +, − | −, −, − | −, +, + | +, −, − | −, −, − |
| 25 | −, −, + | +, −, − | −, −, − | +, −, − | −, −, + | −, +, − |
| 0 | −, −, − | −, −, − | −, −, − | −, −, − | −, −, − | −, −, − |

− means that there was no color change in the reaction mix after addition of the DNA template and heating for 1 hour at 65° C.;
+ means that there was a color change in the reaction mix, from red to yellow, after addition of the DNA template and heating for 1 hour at 65° C.

In FIGS. 8A-8C, a yellow color (lighter) of the reaction mix after 60 minutes of heating at 65° C. indicates a positive detection, while a red color (darker) indicates a negative detection. This experiment was repeated three times and in all of them, the LoD associated with the application of standard pipettors was consistently 500 copies per reaction. When using the drop dispenser devices hereof, however, there was an LoD of 250 copies per reaction in two replicates (FIGS. 8A and 8B), and 125 copies per reaction in another replicate (FIG. 8C). The better LoD of the devices hereof as compared to a standard pipettor could be due to a lower adhesion of the DNA in the liquid holder of the drop dispenser device (as compared to a pipette tip) before adding it to the LAMP tubes. There were no false positives in all replicates.

These results support that the drop dispenser devices hereof can be successfully applied in the detection of bacterial DNA.

### Example 8

LAMP Assays on Samples from Artificially Contaminated Collection Flags Using the Drop Dispenser Device

A number of experiments were conducted to examine the capability of the drop dispenser devices for use with whole-cell LAMP analysis to evaluate if such devices can be a part of a larger kit intended for running on-farm LAMP assays using crude samples. Therefore, the efficacy of the drop dispenser device was evaluated using bacterial cells-instead of just extracted DNA—as the template.

In Wang et al. (2023), supra, it was demonstrated that the use of collection flags (made from plastic sheets) can be better for the collection of bioaerosols as compared to lettuce leaves. These collection flags provided more consistent and reproducible results most likely due to the ease of swabbing flat plastic compared to leaves with grooves and various microstructures. Thus, collection flags were fabricated and manually contaminated with various dilutions of E. coli O157:H7 culture, and then used the swabbed and resuspended cells for the LAMP assays. This approach was a simulation of on-farm assays. Since pathogens were used, the tests with live cells could not be performed outside the laboratory.

Briefly, each collection flag consisted of one piece (5 cm×30 cm) of a transparency film (Apollo Plain Paper Copier Transparency Film, 617993) attached to a wooden stick. For the current in-lab experiments, the flags were not attached to sticks.

Five dilution levels of the bacterial culture (as described in Example 4 above)—with dilution factors of 1, 10, 100, 1000, and 10000—were prepared. For this purpose, 2 mL of the initial bacterial culture in the BHI broth was centrifuged at 9000 rpm for 1 minute. After removing the supernatant liquid, the cells were resuspended and well-mixed in the same amount of water and centrifuged again. After a second resuspension in the same amount of water, the OD₆₀₀ was read using a CLARIOstar microplate reader (BMG Labtech Inc., Ortenberg, Germany) and the resuspended bacteria were used to generate further dilutions. The OD₆₀₀ was translated into bacterial count using a calibration curve (FIG. 4). In addition to the bacterial dilutions, water and bacterial DNA extract (1×10⁴ copies/μL) were also used as negative and positive control templates, respectively.

200 μL of each template (bacterial dilutions, bacterial DNA, and water) was spot inoculated on one side of a collection flag. For each template, three clean flags laid inside a biosafety cabinet were used. Using separate sterile inoculating loops, the inoculums were gently spread over the entire surface of each flag and left to dry out for 60 minutes. Then, using a wet polyester-tipped swab (BD BBL, Franklin Lakes, NJ; 263000), the templates were collected from each flag surface. To do that, a sterile swab was first dipped in molecular biology-grade water. Then, the wet swab was rubbed over the entire surface of the flag and finally resuspended in 200 μL molecular biology-grade water. The obtained samples were used for running LAMP assays in 0.2-mL polymerase chain reaction (PCR) tubes.

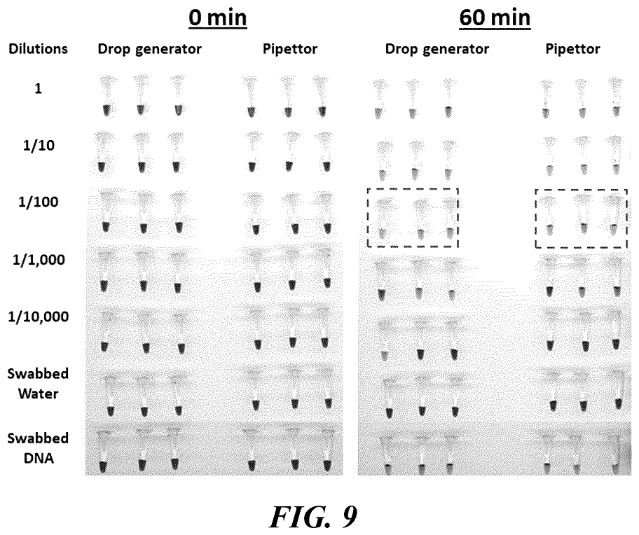

In general, the performance of the drop dispenser device was similar to that of the pipettor (Table 7 and FIG. 9).

TABLE 7
Results of the whole-cell LAMP assays using the
drop dispenser device vs. a standard pipettor.

| Swabbed samples (dilution factor) | Device | Pipettor |
|---|---|---|
| Bacteria^{a} (1) | +, +, + | +, +, + |
| Bacteria (10) | +, +, + | +, +, + |
| Bacteria (100) | +, +, + | +, +, + |
| Bacteria (1000) | +, +, − | −, +, − |
| Bacteria (10000) | −, −, + | −, −, − |
| Water | −, −, − | −, −, − |
| Bacterial DNA extract^{b} | +, +, + | +, +, + |

− means that there was no color change in the reaction mix after addition of the swabbed sample and heating for 1 hour at 65° C.; + means that there was a color change in the reaction mix, from red to yellow, after addition of the swabbed sample and heating for 1 hour at 65° C.
^{a}The initial concentration of the bacteria, Shiga toxin producing E. coli O157 (ATCC 8739) was 7.8 × 10⁸ cells/mL.
^{b}The concentration of bacterial DNA extract was 1 × 10⁴ copies/μL.

The LoD for bacterial detection was 7.8×10⁶ cells/mL (i.e., 1.6×10⁵ DNA copies/reaction).

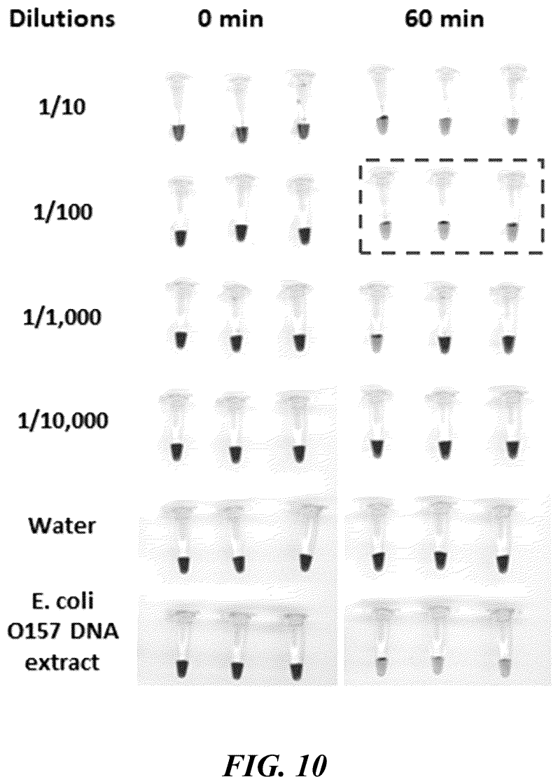

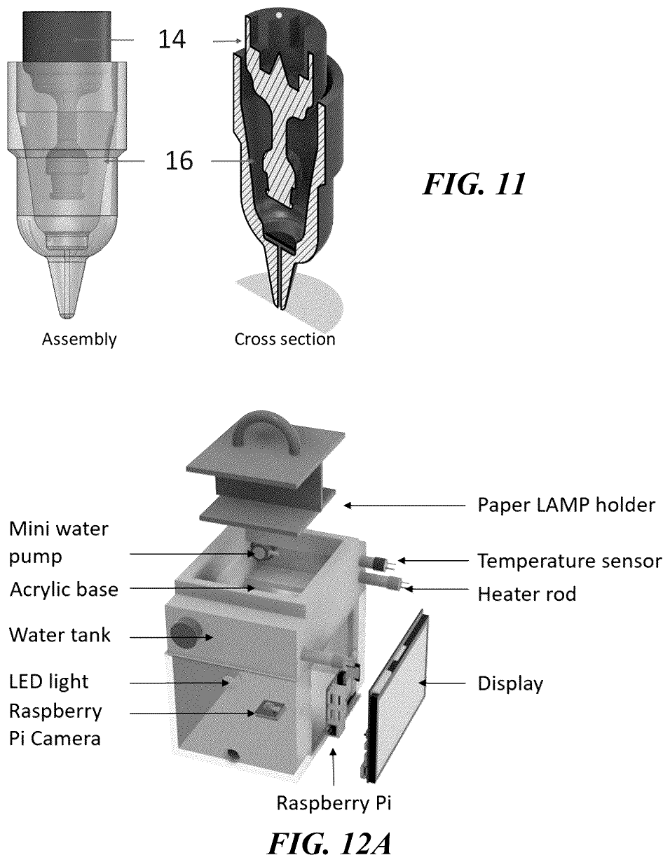

The process of swabbing for microbial collection from artificially contaminated plastic sheets may lose some of the cells as compared to the direct addition of the bacteria from a culture. To investigate this, separate LoD tests were run using bacterial cells (from the same dilutions) directly added to the reaction sites. However, as shown in FIG. 10, the results of this assay were similar to those when swabbing the flags, with a similar LoD of 7.8×10⁶ cells/mL (i.e., 1.6×10⁵ DNA copies/reaction). This highlights the performance of the swabbing technique for on-farm microbial collection using collection flags.

A comparison of the results in Table 7 with those in Table 6 shows that the LoD for LAMP using whole-cell samples was about 3 orders of magnitude worse than when using extracted DNA samples. This might be because in whole-cell LAMP assays, the bacterial genome is not readily available for amplification until the cells are lysed and their membranes are ruptured during the heating process.

### Example 9

### Testing System and LAMP Assay Device Design and Fabrication

A fully integrated LAMP testing system was developed for rapid field detection that integrated heating, imaging, fluid delivery, and a paper-based LAMP assay device. This system facilitated a streamlined “sample-to-result” process for detecting fecal contamination, and delivered quantitative results within one hour of sample collection.

Heating LAMP reactions under water facilitates the homogenous heat transmission required for the reactions to occur. Arumugam et al. (2020); Pascual-Garrigos et al. (2021); Wang et al. (2023), supra; Wong et al. (2018). Based on this, the LAMP testing platform was designed and fabricated with a water bath heater for conducting LAMP assays on farms, with a goal being the creation of a portable platform that can be used in low-resource settings, such as fields where power outlets are not available. The system needed to have a small footprint and require a relatively low power output. Further design considerations were for the system to be compact (in certain iterations, having total dimensions of approximately 164×135×193 mm) and a working power consumption of approximately 20W.

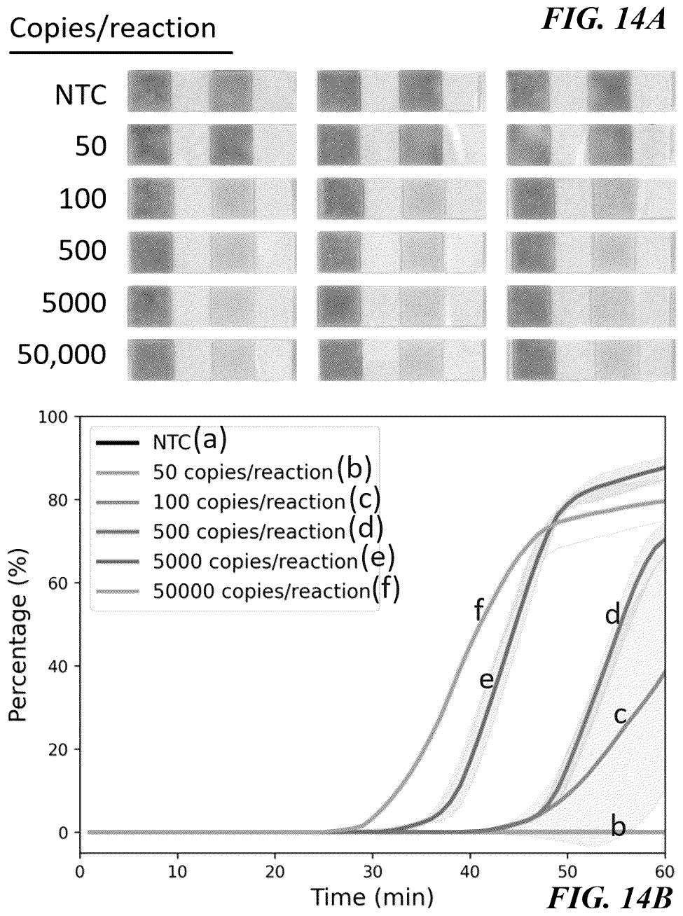

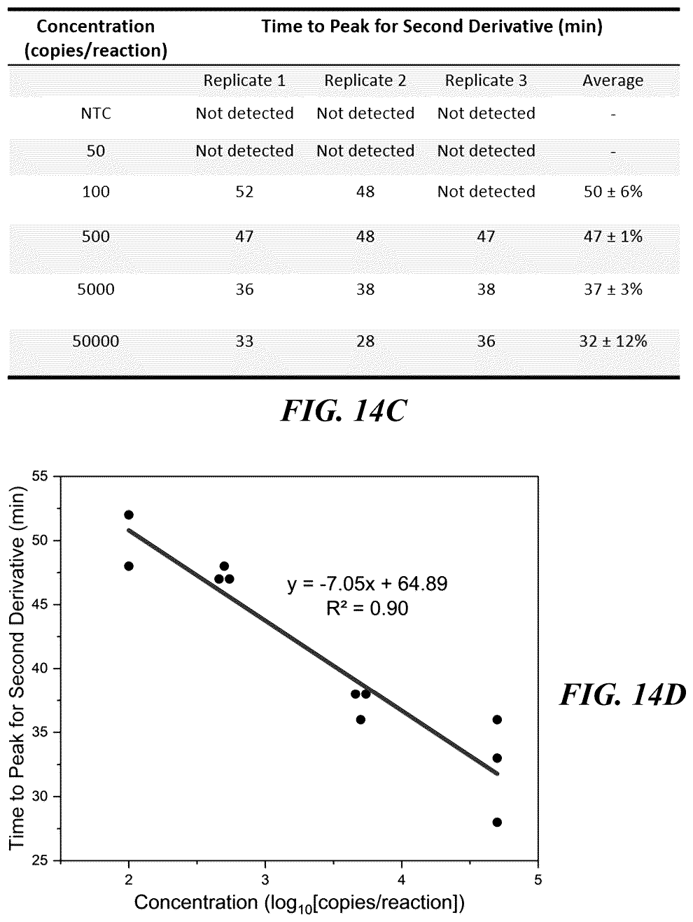

Key features of the system comprise a heating unit (e.g., a water bath) with a transparent observation window, a temperature control system, and an imaging unit for tracking reaction progress (FIG. 12A). Low power consumption allowed for the system to be powered with a portable power bank and the low footprint allowed it to be operated easily from the back of a vehicle in the field. To enable quantification of DNA concentration (rather than just end-point determination of positivity), a camera of the imaging unit was configured to capture time-lapse images periodically (e.g., every minute) and the color change timepoint was used as input for a linear regression equation (FIG. 14D) to quantity DNA concentration for unknown samples.

All designs were prepared using SolidWorks software (SolidWorks, MA). The heating unit consisted of a 3D printed cavity made of Dental LT Clear V2 resin (Formlabs, Somerville, MA; RS-F2-DLCL-02) for the first testing platform device and Biomed Clear VI resin (Formlabs, Somerville, MA; RS-F2-BMCL-01) for the second first testing platform device, with a 3 mm transparent acrylic sheet (Amazon, B099J2XVRW) attached at its bottom.

A platform for holding the imaging components was printed with Grey Pro V1 resin (Formlabs, Somerville, MA; RS-F2-PRGR-01) for a first testing platform device and Raise 3D Premium PLA filament in art white (Raise3D, CA) for a second testing platform device. The first and second testing platform devices had very similar designs, except the second device had a small cooling fan (Amazon, B06XQDMMJ5) installed on the bottom of the platform.

Different printing materials were used due to availability at the time of manufacturing. All resin-based 3D printing was performed using a Form 3B stereolithography 3D printer (Formlabs, Somerville, MA), while filament-based 3D printing was performed with a Raise3D Pro2 Plus 3D printer (Raise3D, CA). The transparent window under the heating cavity allowed for coupling the heating unit with the imaging unit to track the color changes in the reaction pads of the LAMP assay device.

For the heating units, each testing platform device used two 80W, 120 V hot rod Dernord heating elements (Amazon, B08LK9HCWW) to heat the water, and further comprised two 12 V submersible mini water pumps (Amazon, B08RWP6GJF) to circulate water in the tank and improve the temperature uniformity. The water temperature was monitored using a waterproof digital temperature sensor (Gikfun, DS18B20), controlled by a PID control algorithm, and ran on a Raspberry Pi 4B (Amazon, B07TD42S27) minicomputer. The testing platform devices provided fast operation, reaching 65° C. in under 20 minutes.

The imaging unit of each testing platform device was a high-resolution autofocus camera with 16-megapixel imaging resolution (Amazon, B09STL7S88) capable of capturing a shot of the paper pads of the LAMP assay device every minute.

For precise sample handling in field conditions, the drop generator device described in Examples 1-8 above that was capable of delivering consistent 27 μL volumes was used. Surface treatments were employed to the drop dispenser device as described herein to ensure reliability and efficiency. Briefly, the drop dispenser device comprised a liquid holder, a plunger, and two O-rings (Helipal, Airy-Acc-Oring-2.5×6 mm) (FIG. 12B). The liquid holders and plungers were 3D printed using High Temp V2 resin (Formlabs, Somerville, MA; RS-F2-HTAM-02). After printing, the parts were washed with IPA for 30 minutes and cured under UV light at 55° C. for 30 minutes. The tip of each liquid holders was wrapped in Parafilm™ wrapping film prior to surface treatment (Fisher Scientific, S37440). The liquid holders were surface-treated with oxygen plasma for 2 minutes at 0.2 Torr using a plasma generator (Plasma Etch, Inc., PE-25) and PEG 400 (Fisher Scientific, P167-1) for 24 hours. Following surface treatment, all components were washed with IPA, then with ultrapure water (PURELAB flex, ELGA), dried with an air gun, and stored in separate resealable polypropylene bags (Uline, S-17954) for future use.

As part of the testing system field-based detection, microfluidic, paper-based LAMP assay devices (μPADs) (FIG. 12C) comprising a specialized colorimetric LAMP reaction mix were employed. The μPADs were fabricated using chromatography paper with polystyrene spacers on an optically clear polyester backer for support.

Each LAMP assay device consisted of two paper pads of 5 mm×6 mm chromatography paper (Ahlstrom-Munksjo, Grade 222) attached on a double-sided adhesive (Adhesives Research, 90178) and a MELINEX (Tekra MELINEX® 454 Polyester (PET)) for support. The paper pads were separated by 5 mm polystyrene spacers (Tekra, 40047020).

The LAMP reaction mix reported by Wang et al. was employed (Wang et al., 2021) in each LAMP assay device. Briefly, to prepare 1000 μL 2× LAMP mix, the solution consisted of 100 μL KCl (1000 mM; Sigma-Aldrich, P9541), 160 μL MgSO₄ (100 mM; Sigma-Aldrich, M2773), 280 μL deoxynucleotide triphosphate (dNTP) (10 mM; Fisher Scientific, FERR0182), 2.8 μL deoxyuridine triphosphate (dUTP) (100 mM; Fisher Scientific, FERR0133), 0.4 μL Antarctic Thermolabile UDG (1 U/μL; New England Biolabs, M0372S), 5.4 μL Bst 2.0 DNA Polymerase (120 U/μL; New England Biolabs, M0537M), 20 μL phenol red solution (25 mM; Sigma-Aldrich, P3532), 100 μL tween 20 (20%; Sigma-Aldrich, P9416), and 331.4 μL nuclease-free water (Fisher Scientific, 43-879-36). After mixing, using a micro-pH electrode (Fisher Scientific, 11-747-328), the pH was adjusted to 7.8-7.9 using KOH (0.1 M or 1 M), to get a red solution.

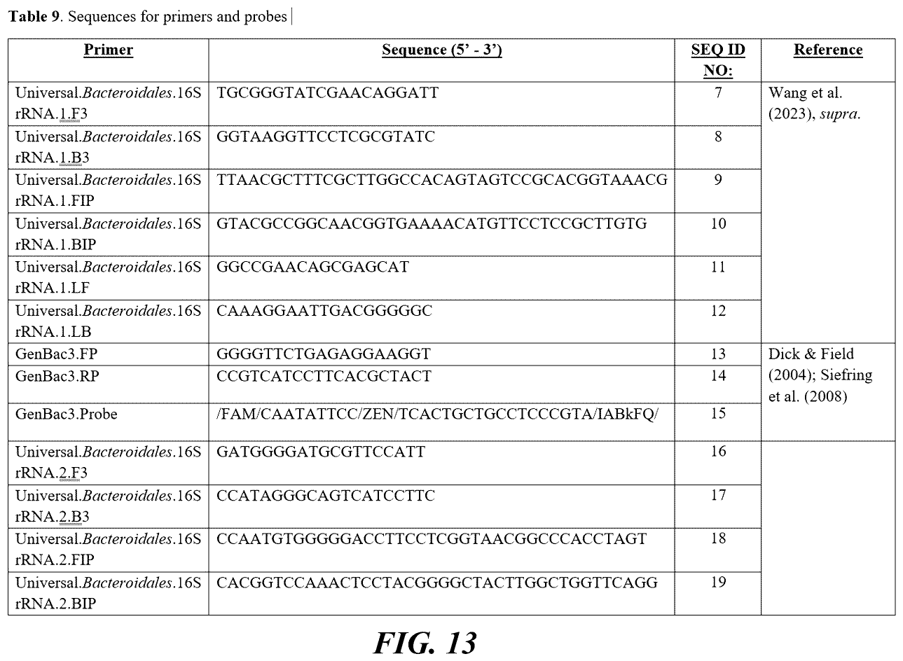

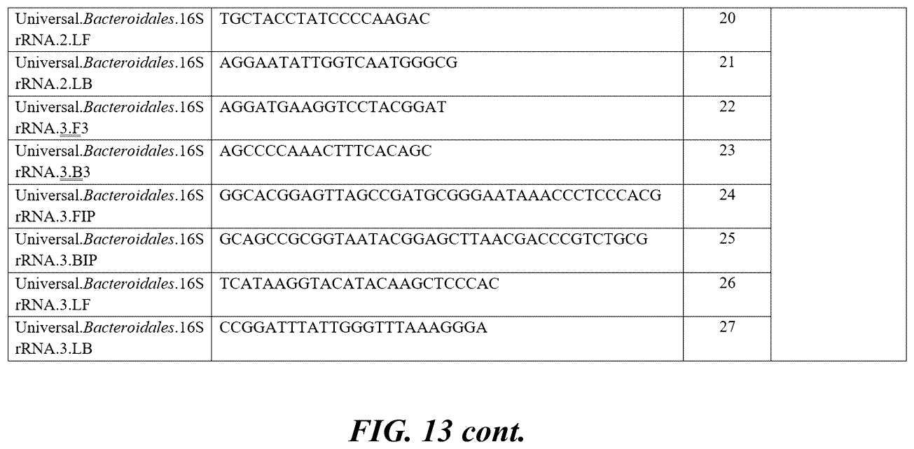

To prepare 200 μL LAMP master mix, 125 μL of the 2× LAMP mix, 25 μL of 10× primer mix (16 μM FIP/BIP, 2 μM F3/B3, 4 μM LF/LB) (final concentration 1.6 μM FIP/BIP, 0.2 μM F3/B3, 0.4 μM LF/LB) (Table 9 in FIG. 13), 0.67 μL Bst 2.0 DNA Polymerase, and 1 μL of betaine (5 M; Sigma-Aldrich, B0300-5VL), 3.13 μL bovine serum albumin (BSA) (40 mg/ml; Sigma-Aldrich, A2153), 36.0 μL trehalose (50% (w/v); Thermo Scientific Chemicals, 182550250), and 9.2 μL nuclease-free water were combined and mixed. The pH of the master mix was adjusted to about 7.8-7.9 using KOH (0.1 M), to get a red solution. A 30 μL of the final master mix was added to each paper pad and left inside a PCR workstation (Mystaire, MY-PCR32) to dry for 2 hours (temperature: 20° C.; relative humidity: 39%).

For each μPAD, one paper pad was loaded with the LAMP primer mix (i.e., the reaction pad) and the other one without the primer mix (no-primer control pad). When no primer mix was used, the same volume of nuclease-free water was added to the mix instead. The dried μPADs were packed in separate reclosable polypropylene bags (Uline, S-17954) and stored at −18° C. until usage. μPADs used for the field test were shipped to the field (Salinas, CA) and kept in the freezer of a household refrigerator (−18° C.) until usage.

After fabrication, the μPADs were stored in resealable polypropylene bags (Uline, S-17954) for future use.

### Example 10

### LoD of LAMP Assay Devices

LoD experiments were performed on μPADs to evaluate the sensitivity of the assay (FIG. 14). 5 levels of swine stool DNA extract concentrations were used: 50,000, 5,000, 500, 100, and 50 copies per reaction, stock quantified using dPCR, as well as no template controls (NTC) (FIG. 14A). Briefly, the dPCR reactions were performed in a total volume of 40 μL, containing 10 μL 4× Probe PCR Master Mix (250102; Qiagen, USA) (final concentration 1×), 4 μL of 10× primer-probe mix (final concentration 1×, 0.8 μM forward primer, 0.8 μM reverse primer, 0.4 μM FAM probe) (Table 5), 0.5 μL EcoRI-HF restriction enzyme (NEB, R3101S), 20.5 μL nuclease-free water, and 5 μL of the template. The dPCR reactions were performed in a 26K 24-well Nanoplate (Qiagen, 250001) on a 5-plex QIAcuity One digital PCR instrument (Qiagen, 911021). The thermal cycling conditions were implemented using the following program: initial denaturation at 95° C. for 2 minutes, followed by 40 cycles of 95° C. for 15 seconds, 55° C. for 15 seconds, and 60° C. for 30 seconds. The absolute quantification method was used to calculate the DNA copy number with the QIAcuity Software Suite. The DNA sample was stored at −80° C. until usage. All reactions were done in triplicates.

For the LAMP assays, 27 μL of the template (stool DNA or nuclease-free water for NTC) was added to both paper pads (control pad and reaction pad). The μPADs were separately sealed inside resealable polypropylene bags (Uline, S-17954) and heated at 65° C. for 60 minutes in a water bath (Anova, ANTC01).

Time-lapse video of the μPADs was taken from 0 to 60 minutes using a HERO8 Black digital camera (GoPro, SPJB1). Pascual-Garrigos et al. (2021), supra; Wang et al. (2023), supra. The μPADs were scanned at 60 minutes using a flatbed scanner (Epson, B11B223201) to determine the end-point result. The LoD of the μPADs was 100 copies/reaction (FIGS. 14A-14D).

The time-lapse video images were then processed to quantify the colorimetric result. Briefly, using the OpenCV library, each pixel was classified into weighted color bins to calculate the sample positivity percentages over time. Derivative analysis was used to identify rate changes in positivity over time to calculate DNA concentration in samples.

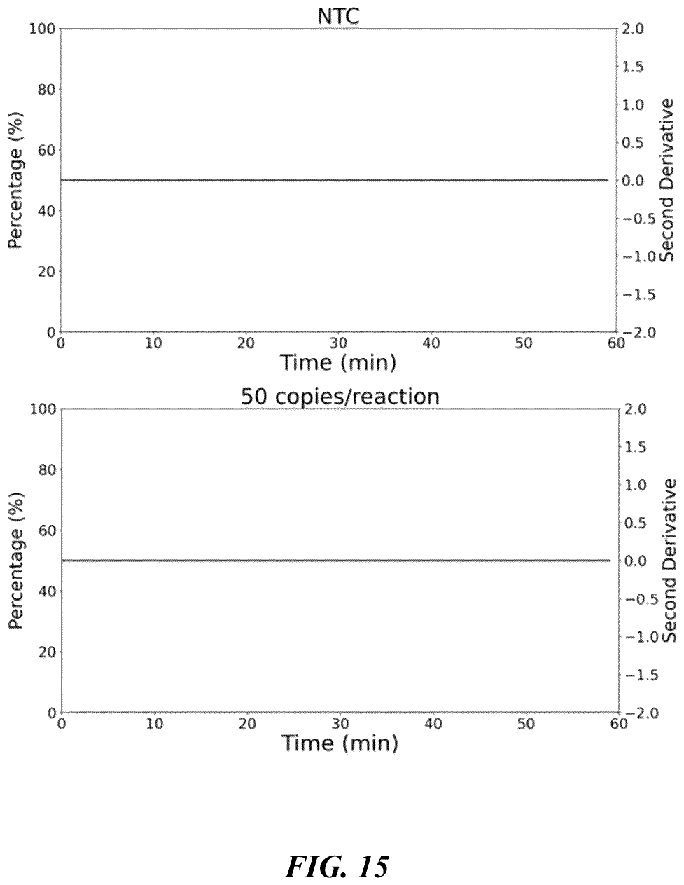

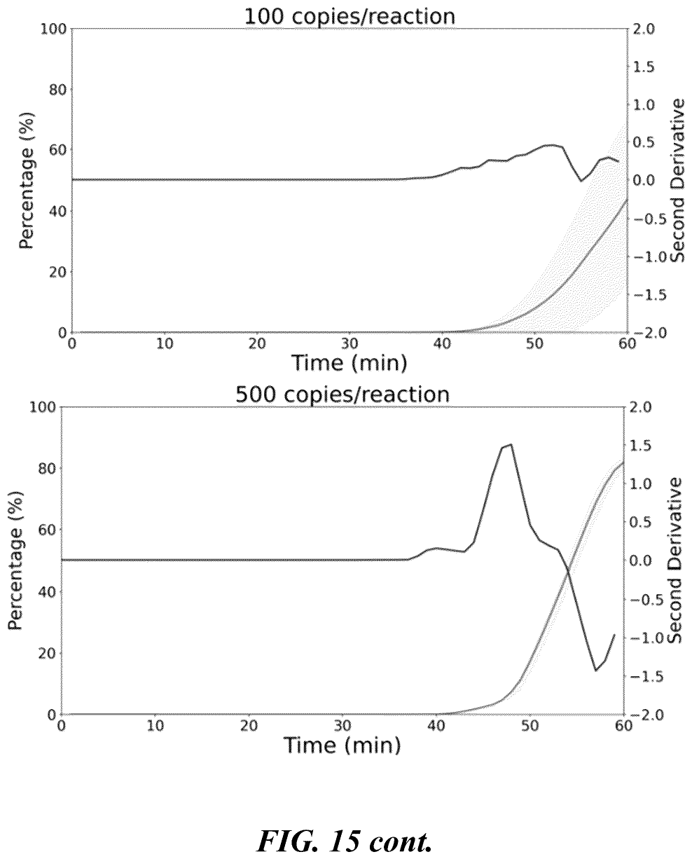

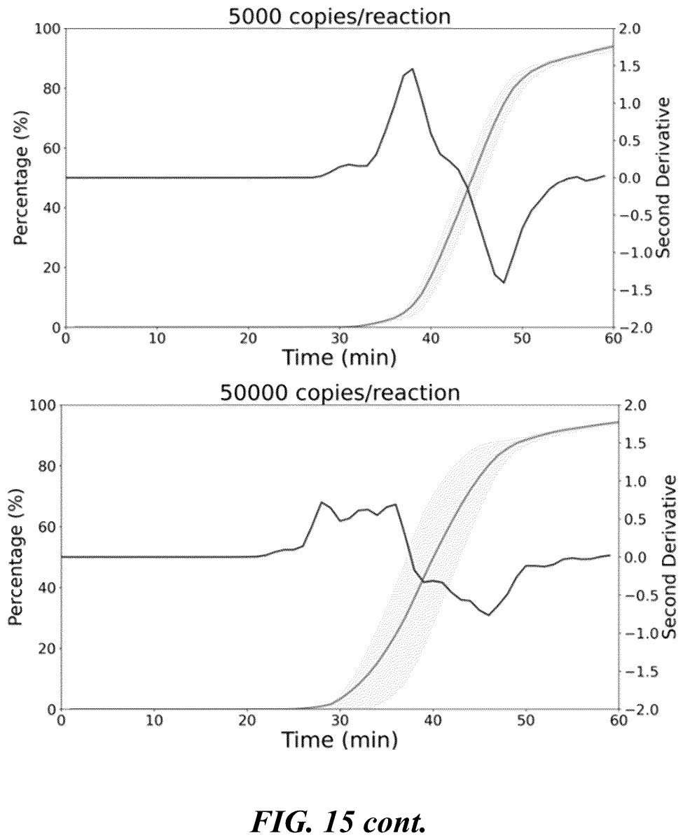

The algorithm calculated the percentage of positivity for each sample as well as the change in positivity over the course of 60 minutes. Following the calculation, a quantitative analysis displaying the positive percentage throughout the test run was plotted (FIG. 14B). The second derivative of FIG. 14B was calculated and the time-to-peak value was adopted as the indicator for sample DNA concentration (FIG. 15). In instances where the mean y-values did not surpass the lowest concentration (100 copies/reaction) signal (0.75% positivity/minute), the amplification curve was considered flat, therefore, the sample was considered negative. Subsequently, the peak values were subjected to a linear regression analysis and the calibration curve was used to measure the concentration of DNA for field samples (FIGS. 14C and 14D).

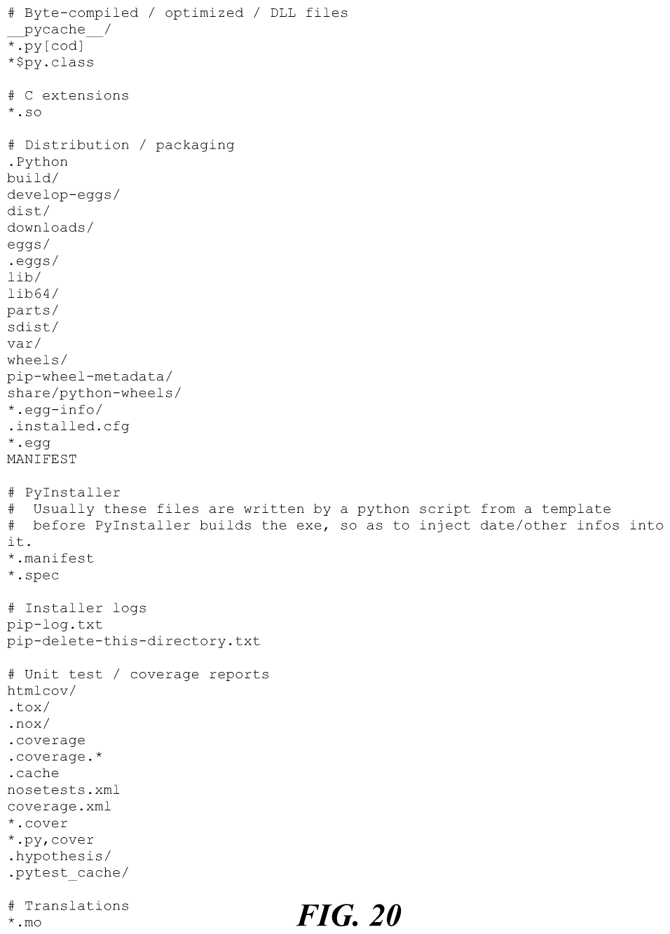

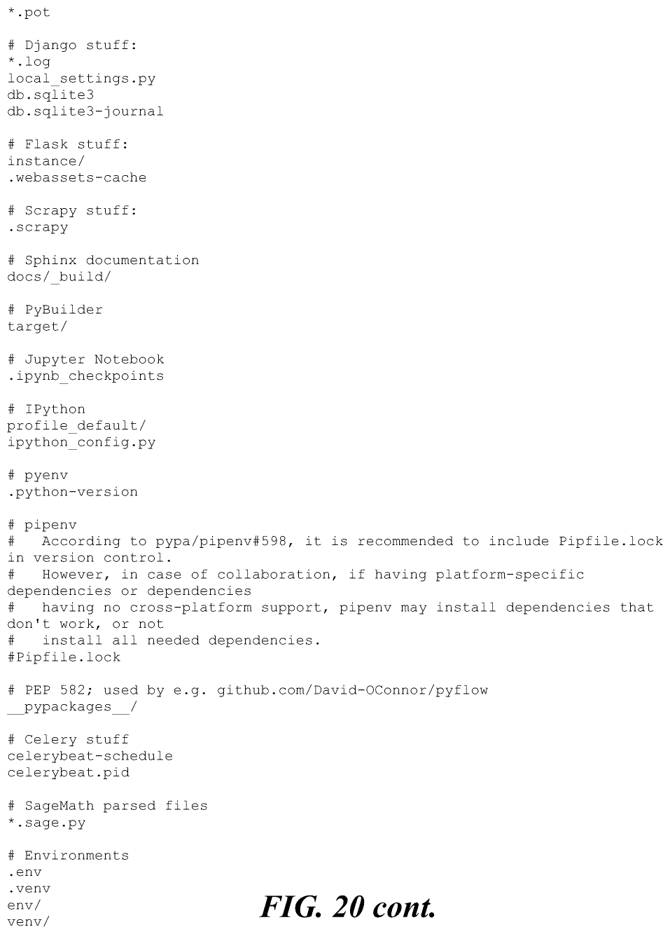

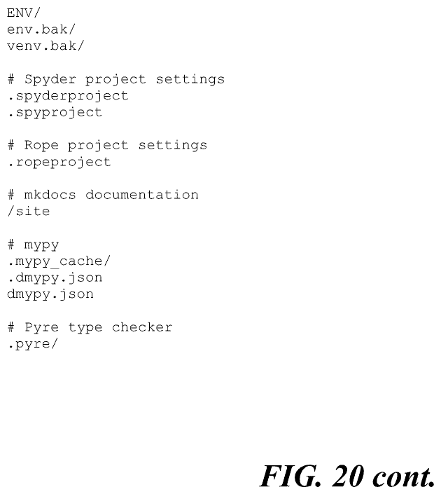

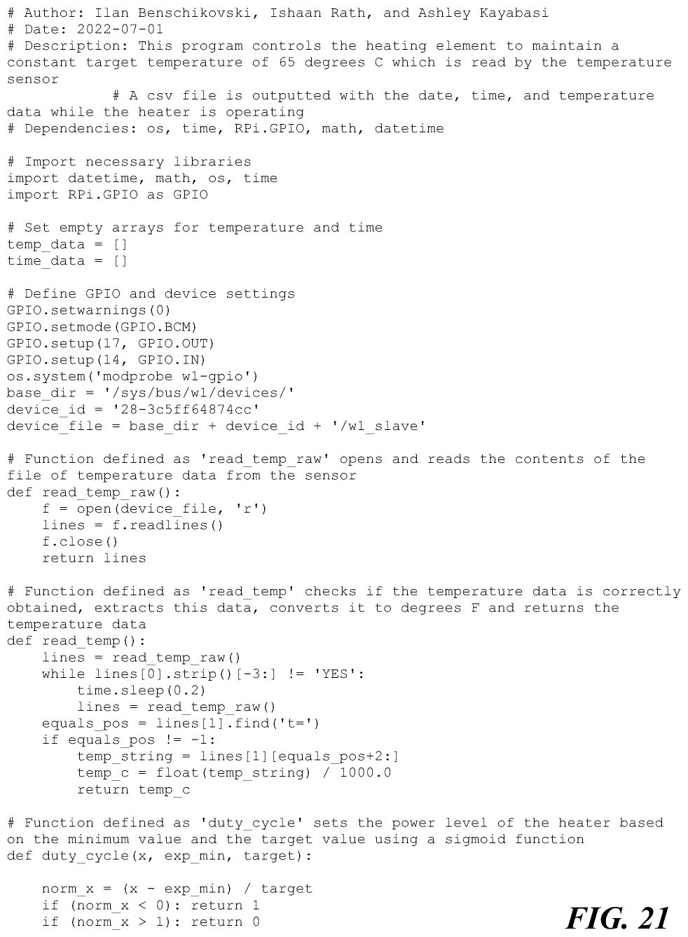

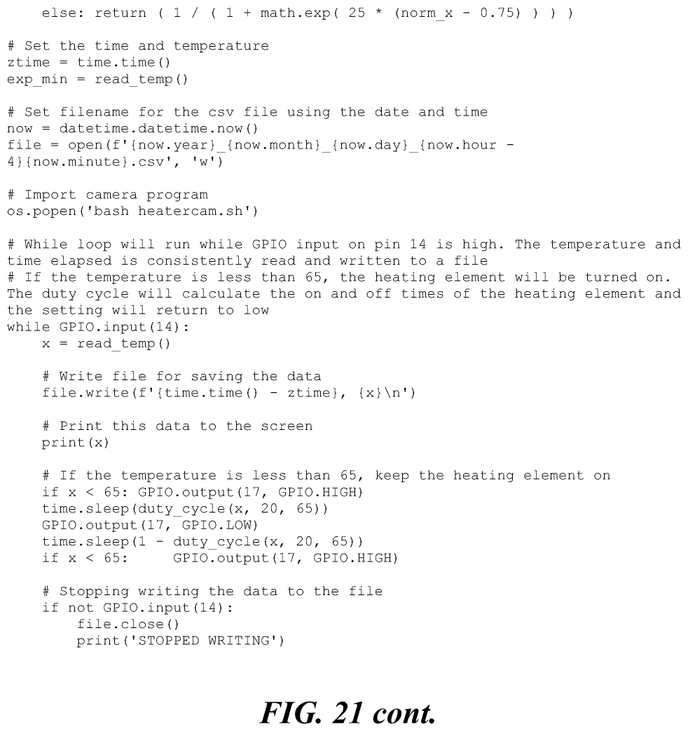

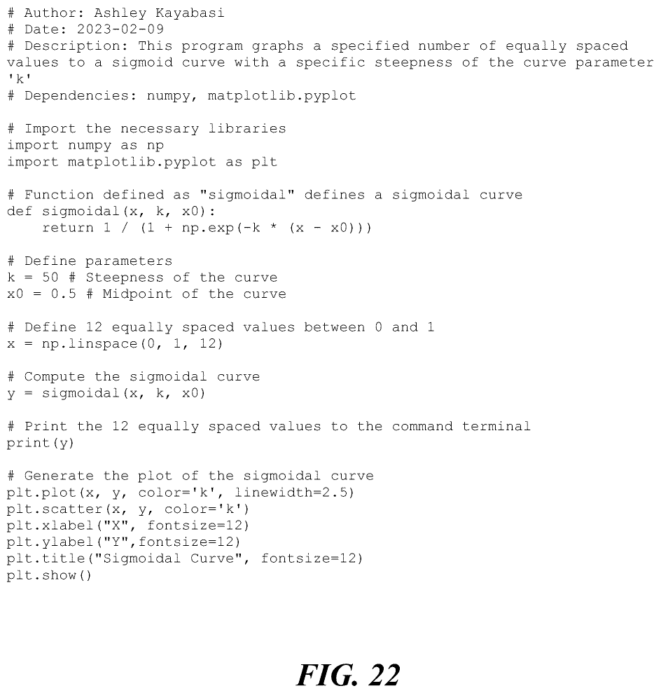

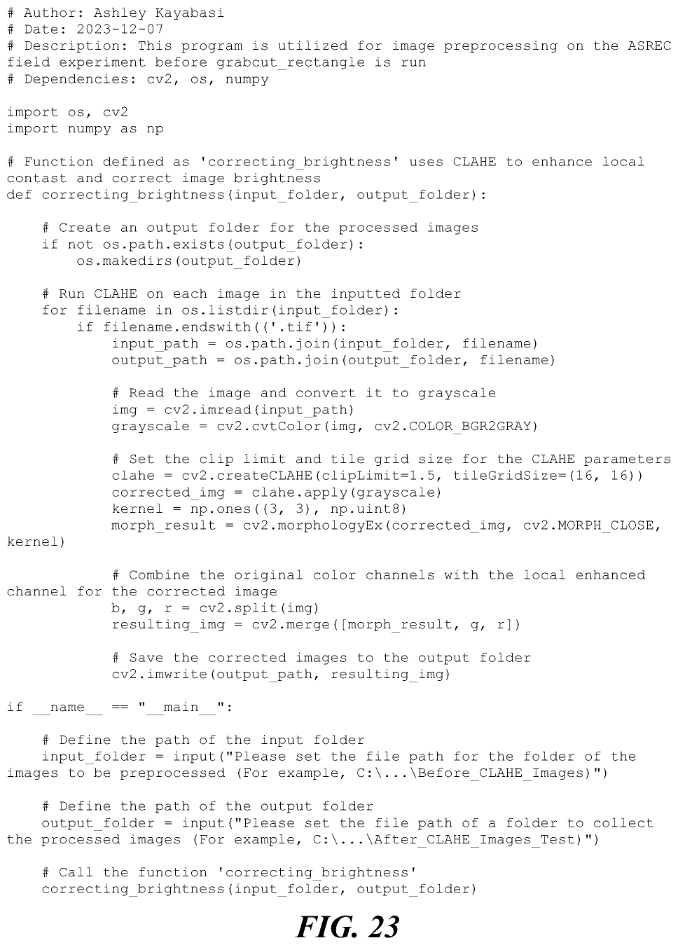

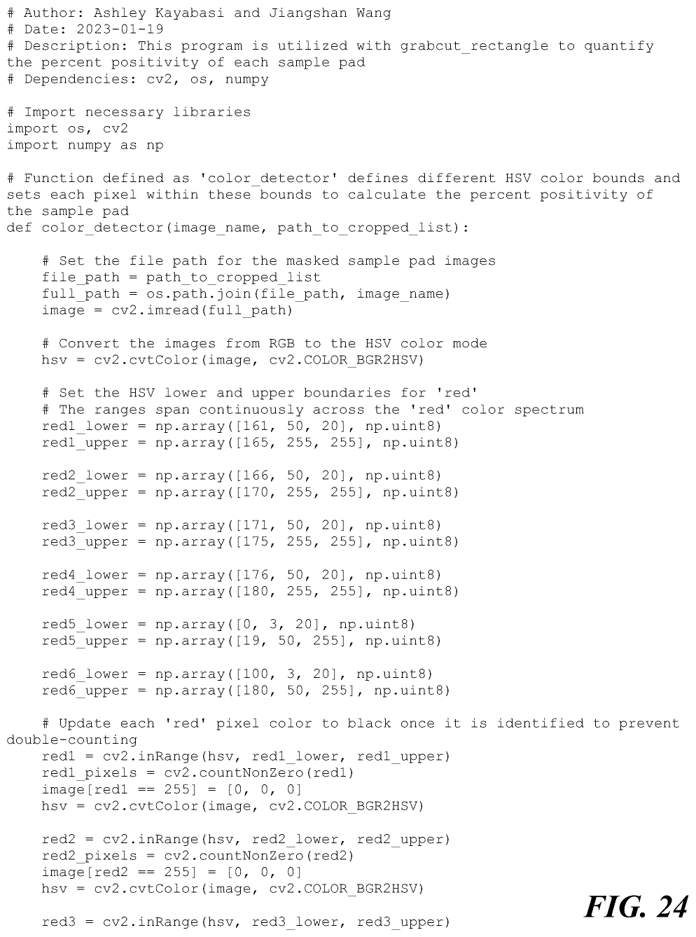

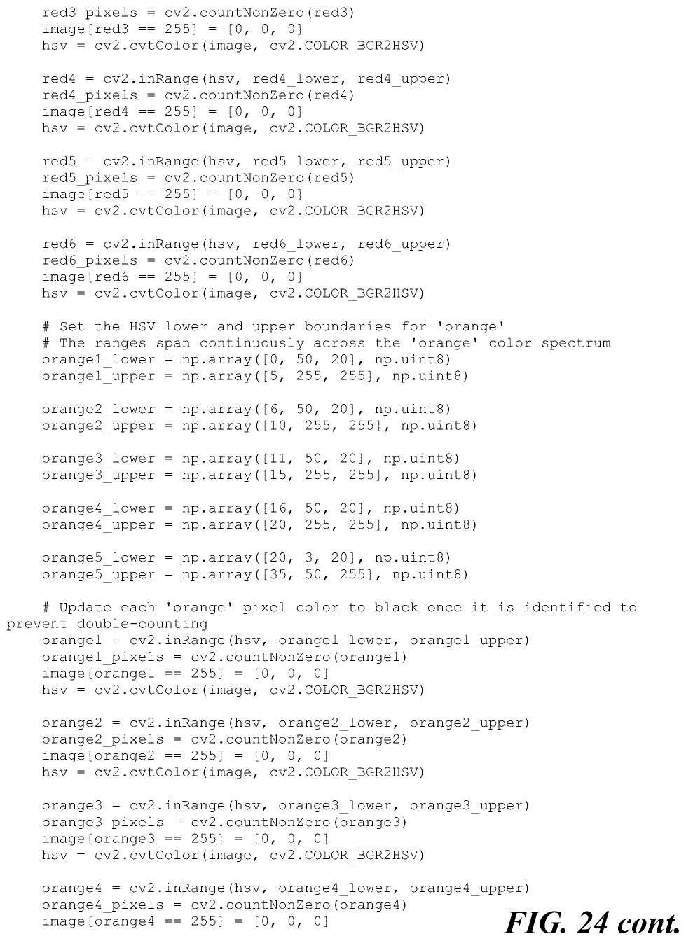

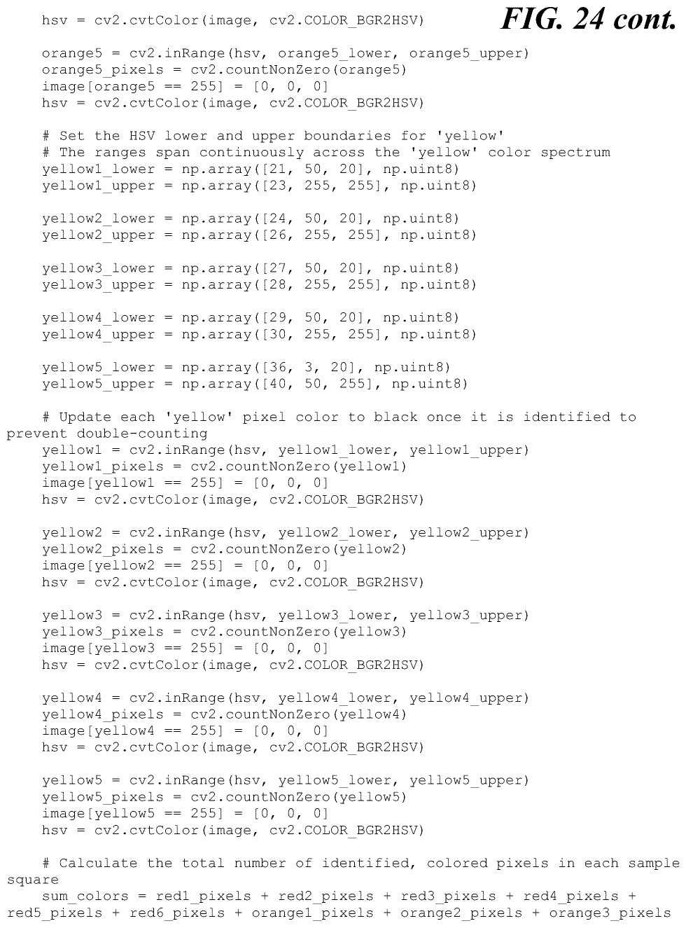

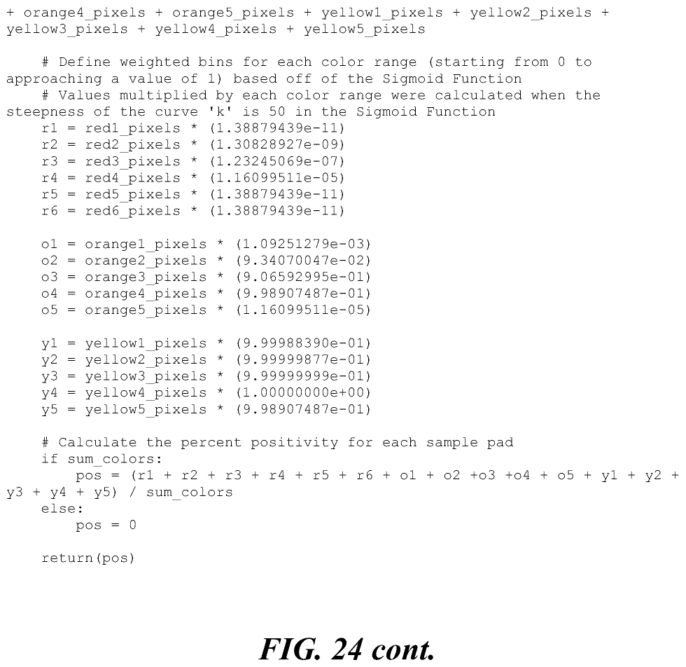

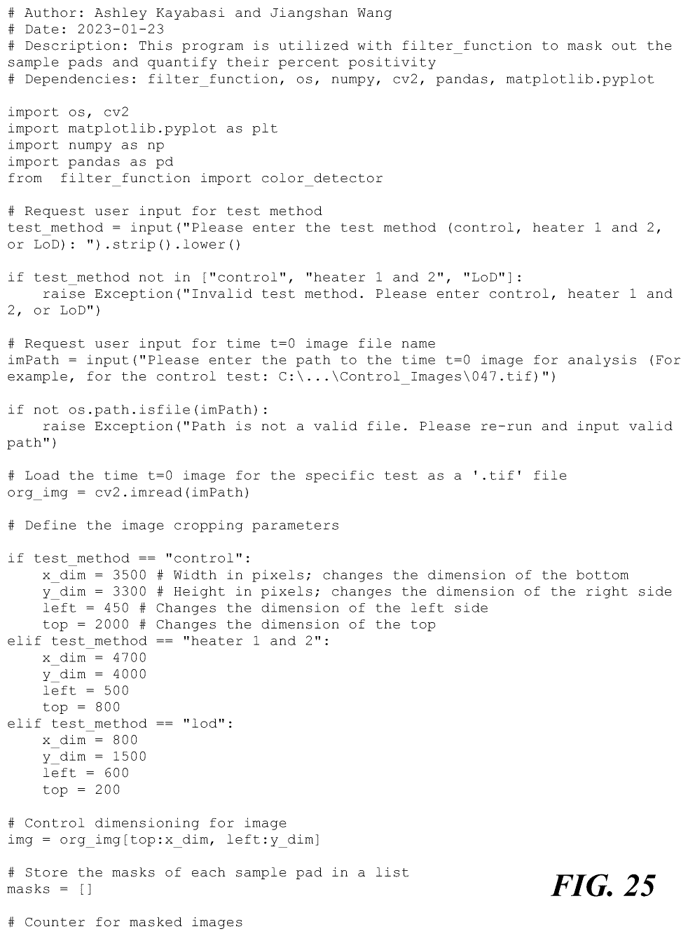

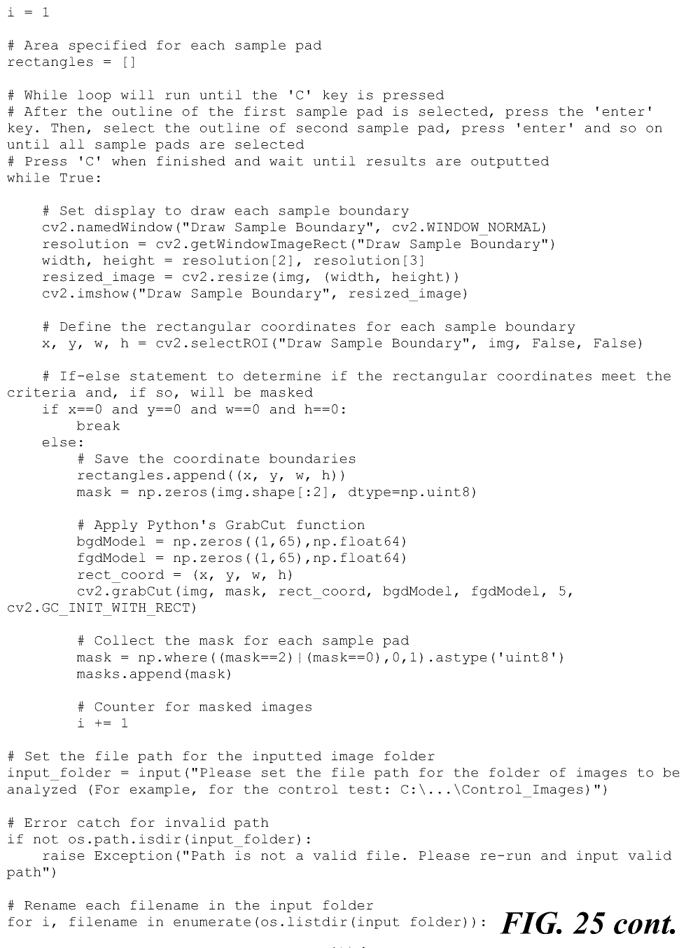

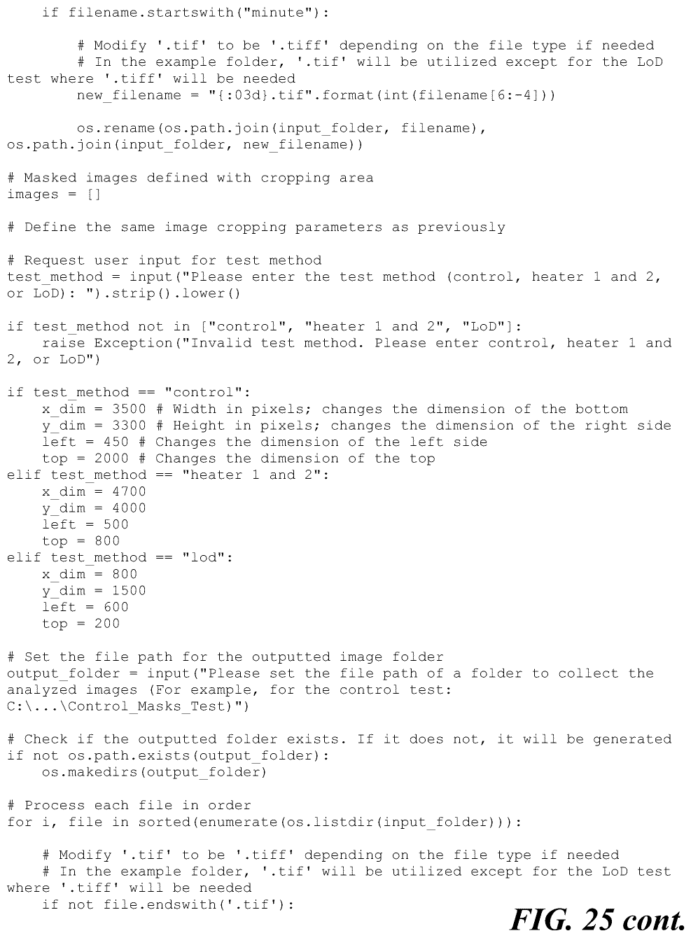

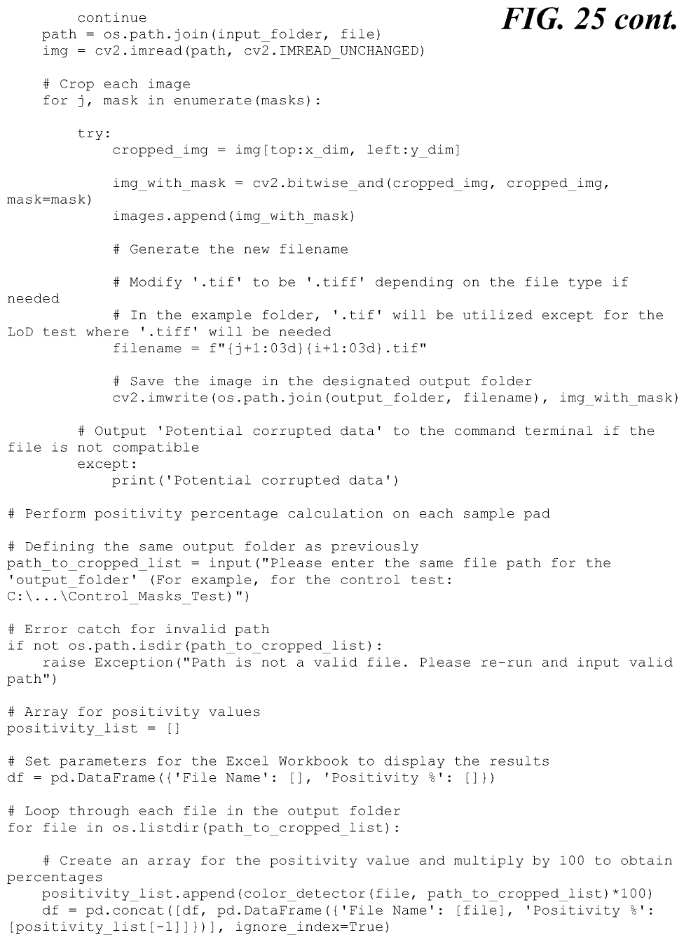

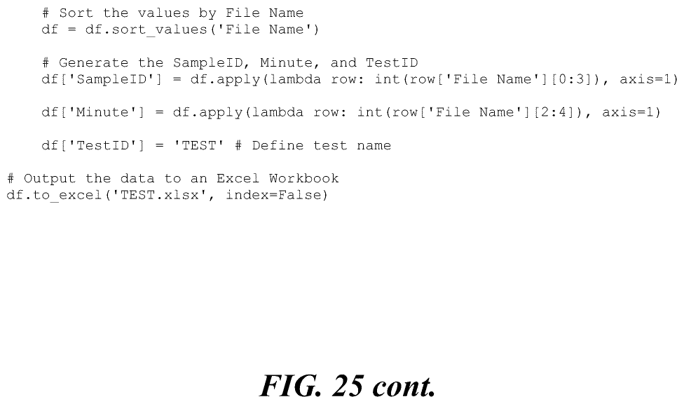

The data that support the findings of this study are publicly available in the FARM-LAMP_Image_Analysis repository at https://github.itap.purdue.edu/VermaLab/FARM-LAMP_Image_Analysis, the contents of which are incorporated herein by reference in its entirety. FIGS. 20-25 are the code related to the above-described algorithms and programs.

### Example 11

### Sampling Collection and Detection of Fecal Contamination

Collection flags (made from plastic sheets) were used to collect microbial samples in the field. These collection flags provided more consistent and reproducible LAMP results compared to lettuce leaves with grooves as noted above. Wang et al. (2023), supra. The investigation extended across two distinct field studies in Salinas, California, at a commercial lettuce farm, and in West Lafayette, Indiana, adjacent to an animal operating unit, to assess environments with varying levels of fecal contamination.

The collection flags were assembled using bamboo skewers (29.8 cm), transparent film (Apollo, VPP100CE), a stapler, and a paper cutter. The transparent film was pre-cut into 7.62 cm×21.59 cm (3 inches×8.5 inches) strips. One piece of the film was stapled together with the bamboo skewer to form a flag.

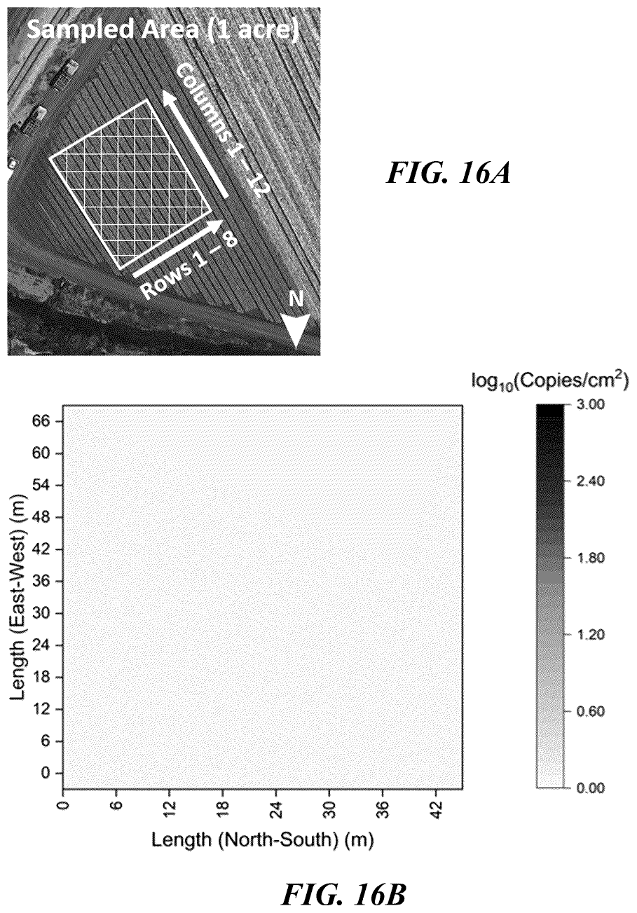

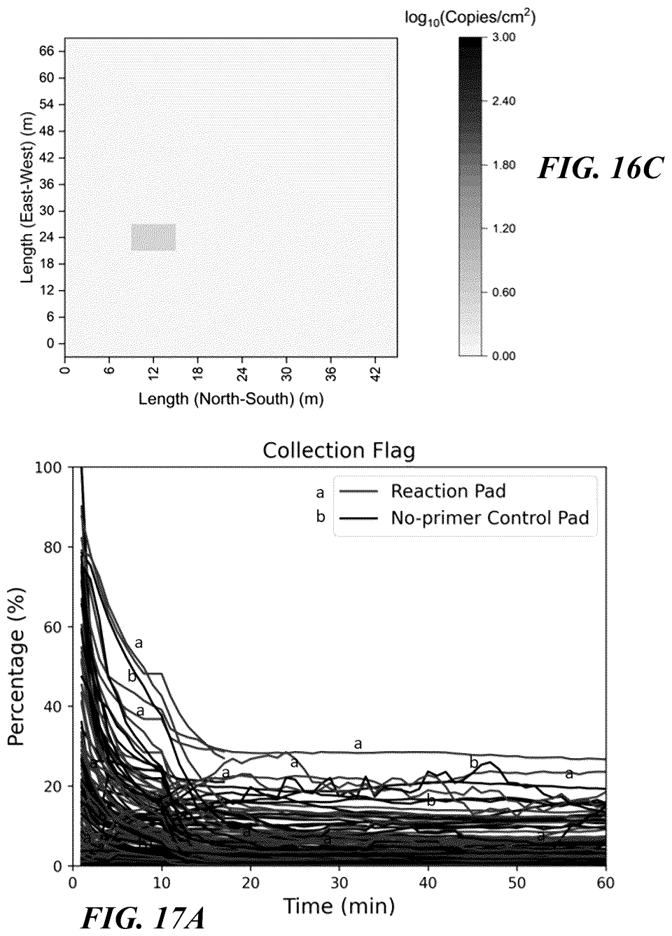

The California commercial lettuce field was labeled with row and column numbers with the distance between each row and column to be 6 meters. Samples were collected at the intersection of each row and column (approximately 100 sampling sites per acre of field) (FIG. 16A). To collect airborne microbiological samples from the field, 96 collection flags were put at each sampling location seven days before sample collection. Each collection flag was encoded with a unique identifier and the location associated with the flag's identifier was recorded.

After 7 days, all collection flags were collected and each flag was placed in an individual, pre-labeled Ziploc resealable storage bag (Amazon, B07NQVYCG3). The samples were collected from both sides of the flag surface (7.62 cm×21.59 cm×2), and all target DNA was resuspended from the swab into 200 μL nuclease-free water. See Wang et al. (2023), supra. To attain a more consistent swabbing, the swab was pre-wet with nuclease-free water before swabbing.

All 96 collection flag samples (swab resuspension) and two positive controls (1 ng/reaction swine stool DNA extract) and two NTCs (nuclease-free water), were added on-site using the drop dispensing device without any additional measures to avoid contamination. With a single press, the drop dispensing device dispensed a high precision of fixed drop volume and added it to the μPADs.

For the California lettuce field, all assays were conducted in the field (in the back of a car) using the portable LAMP testing system described above and powered by a portable power station. The unit was powered by a Jackery Portable Power Station (500 W, 110 V) (Jackery, Explorer 500). For each sample, the collected swab resuspension was directly transferred into a drop generator which was subsequently used to rehydrate each pad with a 27 μL drop. Ranjbaran et al. (2023). The rehydrated μPADs were sealed inside reclosable polypropylene bags (Uline, S-17954) and heated at 65° C. for 60 minutes inside the testing device.

The imaging system took time-lapse photos of the μPADs every one minute during the heating time. Subsequently, all 100 μPADs were manually inspected after the 60-minute reaction period.

The remaining swab resuspension samples from each location were stored in separate 1.5 mL vials, kept on ice, and shipped back to West Lafayette, Indiana in a cooler box with ice packs via FedEx Priority Overnight.

The results of the experiments were converted to copies/cm² for better clarity. For each field sample, the concentration of the reaction (copies/reaction) was determined using the sample's time-to-peak value from the regression line (FIG. 14D). Copies per cm² of the flag surface was then computed by dividing the total swabbed DNA copy number ([copies/reaction]÷sample volume×200 μL) by the total surface area (329 cm²). This method ensured a more straightforward representation of the results in terms of Bacteroidales DNA concentration in the field.

No color change was observed in the NTCs, while the reaction pads of the two positive controls turned yellow. There was no visible color change observed in either the no-primer control pads or reaction pads for the 96 collection flag samples, which indicated that all paper LAMP assays were valid and Bacteroidales were not detected in the fresh produce farms.

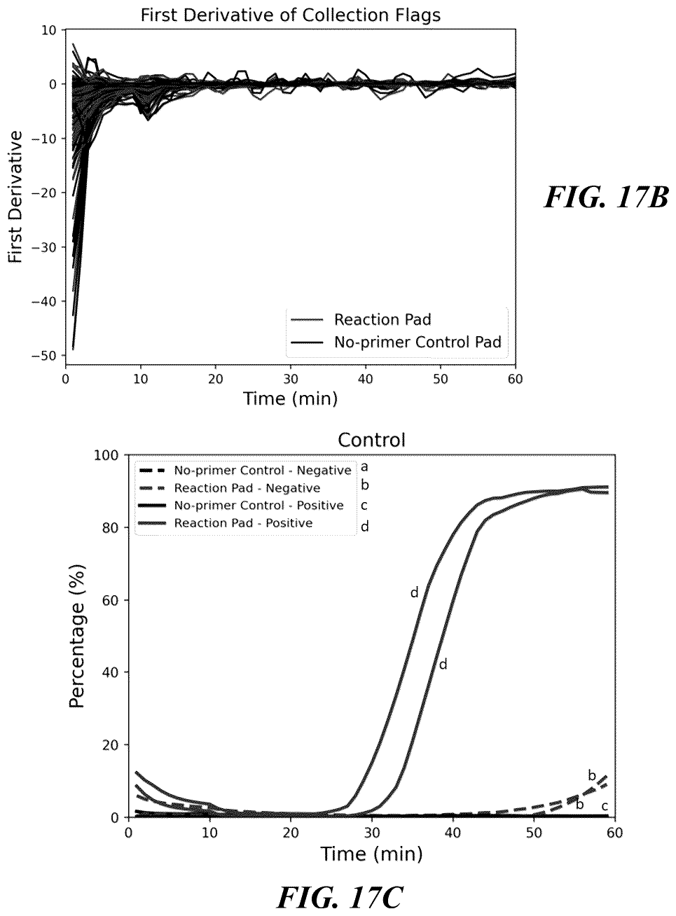

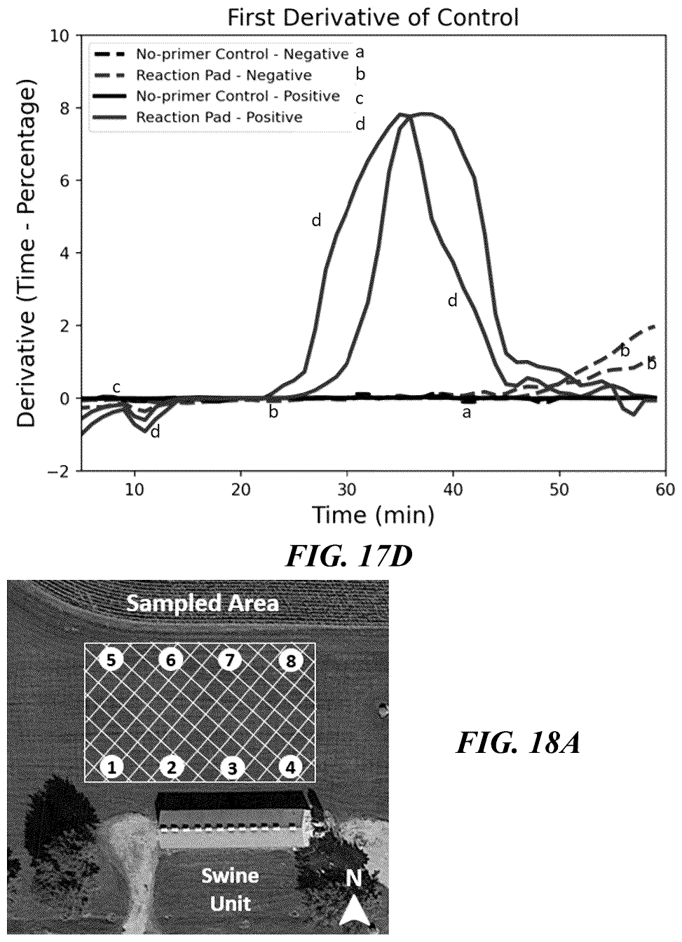

The positivity percentages were plotted to display a qualitative analysis of the color change (FIGS. 17A and 17C). To normalize the data, the first derivatives of the positive percentage over time were then calculated (FIGS. 17B and 17D). Additionally, the second derivative was computed to determine the exact timepoint that marked the beginning of reaction pad amplification. The mean y-values of the first derivative were compared to the mean y-values of the lowest analyte concentration signal (0.75% positivity/min) obtained from the lowest analyte concentration (100 copies/reaction). The mean y-values for all 96 field samples and two negative controls were within the lowest analyte concentration signal level, and the amplification curve was considered flat. Therefore, the time to peak for the second derivative was replaced with the maximum duration of the test run, which was 60 minutes. The two positive controls had a mean y-value of 1.34% positivity/minute (>0.75% positivity/minute) and were determined to be positive by the image analysis algorithm (mean positivity percentage 90.26%). These findings are consistent with the expectation that well-managed commercial fields have a low risk of fecal contamination, and therefore the concentration of Bacteroidales in the environment should be low. However, trace amounts of Bacteroidales DNA may be present in the field due to various reasons, such as the use of organic fertilizer or residuals from previous contamination.

As no positive field samples were obtained from the commercial lettuce field, an additional field experiment was conducted in an open field adjacent to a swine unit at ASREC. A high risk of fecal contamination and a notable concentration of Bacteroidales was anticipated in this area.

For the ASREC field experiment, the process was slightly different than as described with the commercial lettuce field. Similar to the paper-based LAMP assays on the commercial lettuce farm, collection flags were placed in the experimental field for seven days and the assay was conducted on the seventh day. 12 samples, including eight collection flag samples, two positive controls, and two NTCs were tested in the back of a car using the LAMP testing system described.

More specifically, μPADs were enclosed in an updated cartridge. The cartridge (95×80 mm), made of a laser-cut acrylic sheet (Amazon, B08C2JZKNG), has 16 wells separated into two columns, each with dimensions of 6.5×25.5 mm. Each well was designed to hold one individual μPAD strip. The bottom of the cartridge was sealed using a PCR film (ThermoFisher Scientific, AB-0558). After inserting the μPAD strip into the cartridge, 27 μL of the collected swab resuspension was added to rehydrate each pad with a micropipette. The cartridge was then sealed from the top with another piece of PCR film (ThermoFisher Scientific, AB-0558). A white polystyrene sheet (Tekra, Double White Opaque Polystyrene Litho Grade), laser-cut in the same pattern as the acrylic sheet, was placed on top of the upper PCR film. This feature aided in minimizing reflections in the water. The improved design was applied to increase sample throughput within the heater in a single cycle while also preventing water leakage, which may occur with poorly manufactured reclosable polypropylene bags. Bacteroidales concentrations were quantified in the range of 147 to 1042 copies/cm².

qPCR assays were run on the same samples used for the on-farm LAMP assays for both farms using the above-described qPCR protocols. The results of the qPCR assays were compared with those obtained from on-farm LAMP to provide an evaluation of the performance of the on-farm testing system and LAMP assay device.

Briefly, qPCR assays were run on the residual swab resuspension samples from each farm, following protocols for the Luna® Universal Probe qPCR Master Mix (New England Biolabs, M3004). Each 20 μL reaction mix included 10 μL master mix, 0.8 μL PCR forward primer, 0.8 μL PCR reverse primer, 0.4 μL qPCR probe, 7 μL nuclease-free water, and 1 μL template (swab resuspension samples from the farm). The qPCR primers and probe are shown in Table 5. The assays were performed using PCR 96-plates loaded in a qTOWER³ thermocycler (Analytik-Jena, Germany) with an initial denaturation of 95° C. for 60 s and a cycling profile of 95° C. for 15 s, 55° C. for 15 s, and 60° C. for 30 s. The assay has 45 cycles. The results of the qPCR assays were compared with those obtained from on-farm LAMP.

To generate a fecal contamination risk evaluation map of the California commercial lettuce field, the Ct value of each qPCR reaction was converted to log₁₀ (copies/cm²) via a linear fit to log-transformed concentrations. See Wang et al. (2024), supra. The qPCR results showed that all 96 samples had a Bacteroidales concentration of 0-2.24 copies/cm² (FIG. 16C). This result is consistent with the LAMP assay device results, which showed <3 copies/cm² (or <100 copies/reaction).

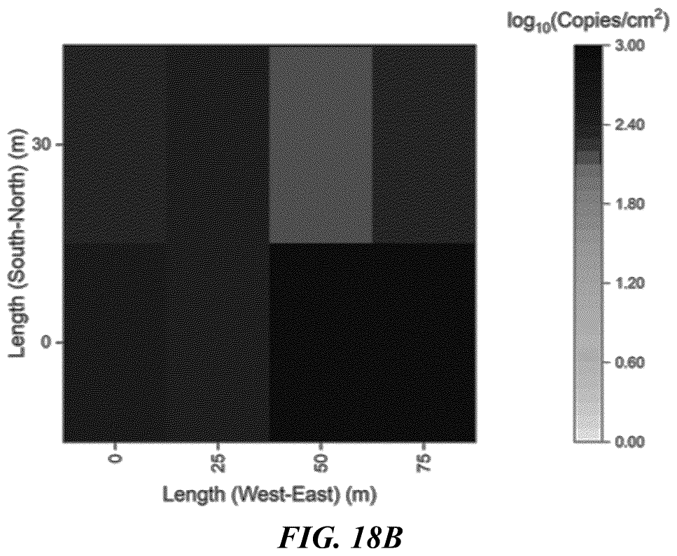

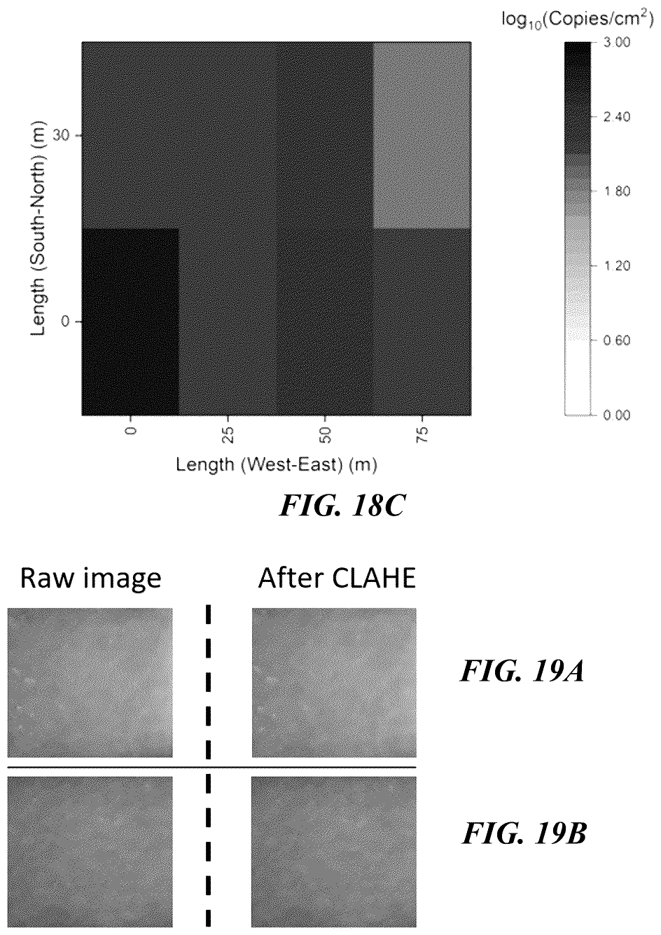

A similar fecal contamination risk evaluation map was produced for the ASREC study (FIG. 18C). The qPCR results showed that all samples were positive, with concentrations ranging from 74 to 657 copies/cm², as compared to 147 to 1042 copies/cm² from the LAMP assay. Some variation was anticipated between the two results because the two assays used different DNA polymerases and had different template inputs (1 μL for qPCR and 27 μL for LAMP). The concentrations obtained from paper LAMP and qPCR were found to be comparable.

The agreement between the two assays indicates that the on-farm LAMP system and assay has reasonable performance for Bacteroidales detection in real-world settings. Although the Bacteroidales LAMP assay did not appear to be as sensitive as qPCR, it was sufficient for identifying possible fecal contamination events in the field. Furthermore, the on-farm LAMP assay offers the advantages of being rapid, low-cost, easy to use, and providing a semi-quantitative result, making it a promising tool for microbial detection in field settings.

The time-lapse photos of all the samples were utilized in downstream image analysis for quantitative analysis and interpretation as described above. Briefly, after the images were captured with the internal camera of the testing system, they underwent a series of image processing techniques beginning with importing and resizing the first image from the test run to reduce processing time. Using Python's OpenCV library, the GrabCut algorithm was utilized to draw the sample boundary for each paper pad and store these rectangular coordinates boundaries to create a mask for each sample. Once these masks were created for the samples in the first image, the program looped through the rest of the test images and created the mask for each sample in each image using the previous rectangular coordinates. The images of the samples were converted to the hue, saturation, value (HSV) color mode and each pixel was separated into weighted bins based on color. The color-coding function set continuous HSV upper and lower boundaries for red, orange, and yellow based on the HSV colormap.

After all pixels were identified and sorted into the different color bins ranging from dark red to light yellow, these bins were weighted based on a sigmoid function with a curve midpoint of 0.5, a curve steepness of 50, and limits of 0 to 1. The program then outputted the percentage of positivity for each sample, by calculating the ratio of the number of pixels in the weighted bins to the total number of identified pixels, to display a quantitative analysis of the color change over time (FIG. 14B).

After calculating these percentages, a quantitative analysis displaying the positive percentage throughout the duration of the test run was plotted using the Matplotlib Python library. To increase the signal-to-noise ratio, a moving average with a window of 10 and a minimum period of 1 was applied. Then, the first and second derivatives of the positive percentage over time were calculated using Python's Numpy gradient function. The mean y-value of the first derivative and the time to peak (the time at which the rate of change is at its maximum) value for the second derivative for each concentration level and replicate were identified. The samples' mean y-values of the first derivative were compared to the mean y-values of the lowest analyte concentration signal (Equations 1 and 2, below). Armbruster & Pry, Limit of blank, limit of detection and limit of quantitation, Clinical Biochemist Reviews 29 (Supp 1): S49-S52 (2008). If the mean y-value of the first derivative was within the lowest analyte concentration signal level, the amplification curve was considered flat, therefore, the time to peak for the second derivative was replaced with the maximum duration of the test run, 60 minutes (FIG. 14C). A calibration curve was generated comparing the time-to-peak for the second derivative and the concentration on a logarithmic scale. The linear regression equation (FIG. 14D) was used to quantify the concentration of DNA for field samples.

$$\\ \begin{matrix}LoD = LoB + 1.645 ( {SD}_{low concentration sample} ) & ( 1 )\end{matrix}$$

$$\\ \begin{matrix}LoB = {mean}_{blank} + 1.645 ( {SD}_{blank} ) & ( 2 )\end{matrix}$$

$$\\ LoB : Limit of Blank ; SD : Standard Deviation$$

Due to variances in lighting, the images captured at the ASREC field experiment were first preprocessed using Contrast Limited Adaptive Histogram Equalization (CLAHE), a filtering technique that aids in image lighting correction. Pizer et al., Adaptive histogram equalization and its variations, Computer Vision, Graphics, & Image Processing 39 (3): 355-368 (1987). FIGS. 19A and 19B illustrate a comparison of the same sample pad coloring in two adjacent time points (22 and 23 minutes) demonstrating a significant color change attributed to environmental factors (e.g., sunset). The application of CLAHE effectively eliminated the warm yellow filter from the images, resulting in a reduction in false color variability across different time points. The subsequent procedure aligns with the image analysis process described previously. CLAHE has been utilized in numerous medical imaging applications and enhances local image contrast while limiting noise amplification by reallocating brightness values in smaller sections of the image. Sonali et al., An approach for de-noising and contrast enhancement of retinal fundus image using CLAHE, Optics & Laser Technology 110:87-98 (2019). CLAHE depends on two different parameters for image correction: the clip limit and the number of tiles. The clip limit represents the contrast factor while the number of tiles, or tile grid size, splits the image into rectangular regions wherein the contrast will be enhanced. Zuiderveld, VIII.5.-Contrast Limited Adaptive Histogram Equalization, In Graphics Gems, P. S. Heckbert (Ed.): 474-485 (1994). For this experiment, a moderate clip limit value of 1.5 was chosen since the images had significant variances in contrast. Additionally, the number of tiles was set (16,16) due to the significant variances in image brightness and to enhance the image contrast in larger regions of the overall image. After, the previous steps in this image analysis process section were followed through.

### Example 12

### Establishing Background Levels of Fecal and Pathogenic Contamination in the Field

Bacteroidales provide an indication of fecal contamination. Certain features of Bacteroidales make them superior to other FIB including, for example, their high prevalence in feces (constituting 30%-40% of total fecal bacteria, 10⁹ to 10¹¹ colony forming units (CFU)/g), obligate anaerobicity (preventing their growth and multiplication in the ambient environment), low natural abundance from non-fecal sources, and high host-specificity (various sequences of the 16S rRNA gene have been designed to detect fecal pollution from specific hosts). Mascorro et al. (2018), supra; Ordaz et al. (2019), supra. Accordingly, Bacteroidales can serve as a valuable—and potentially quantitative-marker to assess the risk of contamination in each farm. However, the levels of Bacteroidales naturally present in the environment of various fresh produce operations, as well as the concentration near animal feeding operations, were not heretofore known. qPCR, the gold standard test for nucleic acid-based assays, was used to establish background levels of fecal contamination in both fresh produce operations and fields near animal feeding operations.

Three different sampling strategies were evaluated in collecting bacterial samples including: 1) swabbing the leaves and resuspending the swab in water (method 1); 2) washing the leaves and using the wash water as a microbial sample (method 2); and 3) using collection flags (clean plastic sheets) to capture bacterial samples and swabbing the flags (e.g., pursuant to the protocols described in International Patent Application Publication No. WO 2023/225573) (method 3).

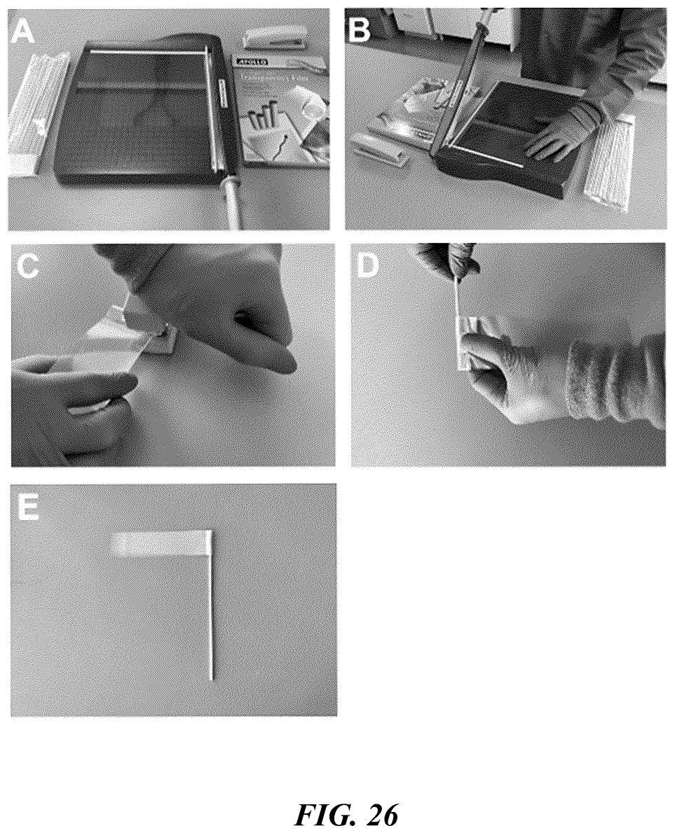

The collection flags were assembled using bamboo skewers (29.8 cm), transparent film (Apollo Plain Paper Copier Transparency Film), a stapler, and a paper-cutter (FIG. 26). The transparent film was pre-cut into 7.62×21.59 cm (3×8.5 inch) strips. Four pieces of film were stapled together at the edge to form a loop. A bamboo skewer was inserted through the loop to make a collection flag. The methods were compared with each other to assess which approach was more reliable in the field experiment.

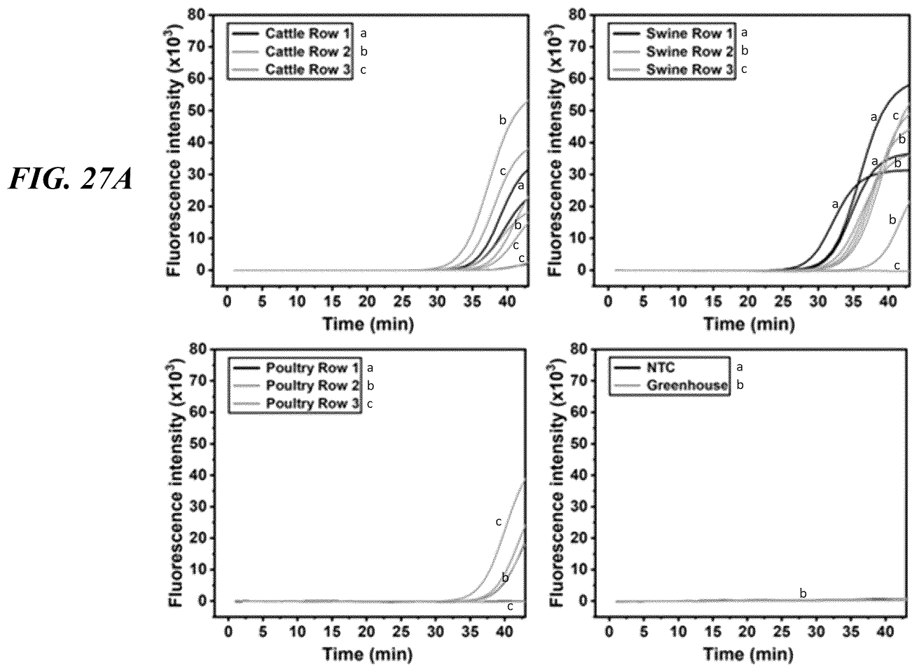

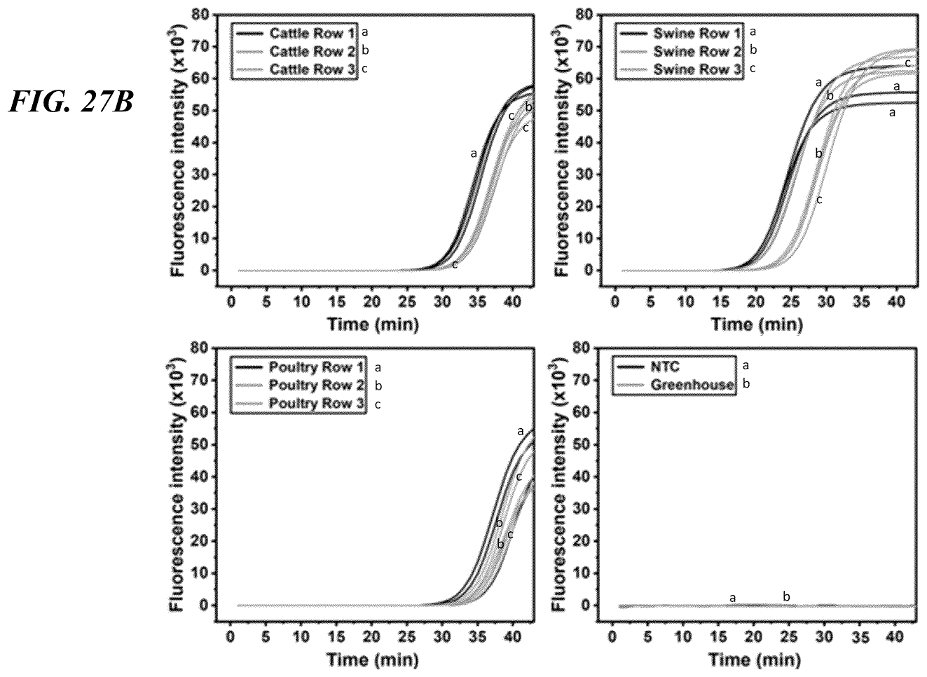

Ten lettuce plants and ten collection flags (per spot) were placed at three different distances (distance varies circumstantially due to the availability of space around animal units) away from each animal operation facility, with three replicates in each row (FIG. 27). Both the plants and collection flags were encoded with a unique identifier and the location associated with the plant/flag's identifier was recorded. A group of ten lettuce and ten collection flags were placed in the greenhouse, which served as the negative control. After 7 days, all lettuce and collection flags were collected. Following United States Food and Drug Administration (FDA) Bacteriological Analytical Manual (BAM) for isolating specific pathogens from fresh vegetable samples, 25 grams of lettuce (approximately four leaves) or four pieces of transparency films were swabbed using a wet polyester-tipped swab (263000, BD BBL, USA). Each swab was resuspended in 200 μL molecular biology grade water. The resuspension was directly used for qPCR without performing DNA extraction. The qPCR primers information is shown in Table 10.

TABLE 10
Sequences for qPCR primers and probes

| | | | SEQ | |
|---|---|---|---|---|
| | | | ID | |
| Target | Primer | Sequence (5′-3′) | NO: | Reference |
| Universal | GenBac3.FP | GGGGTTCTGAGAGGAAGGT | 13 | Siefring et |
| Bacteroidales | GenBac3.RP | CCGTCATCCTTCACGTACT | 14 | al. (2008), |
| | GenBac3. | /FAM/CAATATTCC/ZEN/TCAC | 15 | infra. |
| | Probe | TGCTGCCTCCCGTA/IABKFQ/ | | |
| Cattle | CowM2.FP | CGGCCAAATACTCCTGATCGT | 28 | Shanks et al. |
| specific | CowM2.RP | GCTTGTTGCGTTCCTTGAGATAAT | 29 | (2009), |
| Bacteroidales | CowM2. | /FAM/AGGCACCTA/ZEN/TGTCC | 30 | infra. |
| | Probe | TTTACCTCATCAACTACAGACA/ | | |
| | | IABKFQ/ | | |
| Pig specific | Pig-2- | GCATGAATTTAGCTTGCTAAATTTG | 31 | Mieszkin et |
| Bacteroidales | Bac41F | AT | | |
| | Pig-2- | ACCTCATACGGTATTAATCCGC | 32 | al. (2009), |
| | Bac163Rm | /FAM/TCCACGGGA/ZEN/TAGCC/ | 33 | |
| | Pig- | BIAkFQ/ | | infra. |
| | 2Bac113MGB | | | |
| Human | HF183F | ATCATGAGTTCACATGTCCG | 34 | Bernhard & |
| specific | BFDRev | CGTAGGAGTTTGGACCGTGT | 35 | Field |
| Bacteroidales | BFDFAM | /FAM/CTGAGAGGA/ZEN/AGGTC | 36 | (2000), |
| | | CCCCACATTGGA/IABKFQ/ | | supra; |
| | | | | Converse et |
| | | | | al. (2009), |
| | | | | infra. |
| Poultry | qCD362_F | AATATTGGTCAATGGGCGAGAG | 37 | Kobayashi |
| specific | qCD464_R | CACGTAGTGTCCGTTATTCCCTTA | 38 | et al. (2013), |
| Bacteroidales | qCD394_P | /FAM/TCCTTCACG/ZEN/CTACT | 39 | infra. |
| | | TGG/IABKFQ/ | | |

Siefring et al., Improved real-time PCR assays for the detection of fecal indicator bacteria in surface waters with different instrument and reagent systems, J Water & Health 6 (2): 225-237 (2008); Shanks et al., Quantitative PCR for detection and enumeration of genetic markers of bovine fecal pollution, Applied & Environmental Microbiology 74 (3): 745-752 (2008); Mieszkin et al., Estimation of pig fecal contamination in a river catchment by real-time PCR using two pig-specific Bacteroidales 16S rRNA genetic markers, Applied & Environmental Microbiology 75 (10): 3045-3054 (2009); Converse et al., Rapid qPCR based assay for fecal Bacteroides spp. As a tool for assessing fecal contamination in recreational waters, Water Research 43 (19): 4828-4837 (2009); Kobayashi et al., Effects of temperature and predator on the persistence of host-specific Bacteroidales-Prevotella genetic markers in water, Water Science & Technology 67 (4): 838-845 (2013).

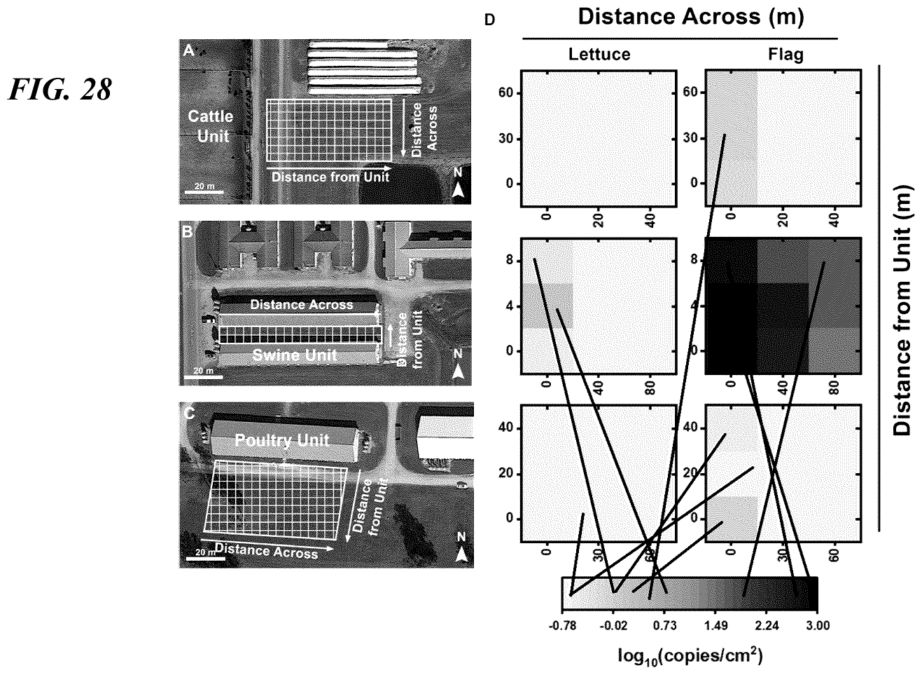

To construct a fecal contamination risk evaluation map, the cycle threshold (Ct) value of each qPCR reaction was converted to log₁₀ (copies/cm²) via a linear fit to log-transformed concentrations (FIG. 28). The heat map demonstrated there were more than 103 copies of Bacteroidales cells per cm² around animal operations (FIG. 28). It was also determined that collection flag samples have higher consistency than lettuce swab samples. Some of the swab samples from lettuce placed next to animal units did not amplify, and the amplification curves had high variability in the time-to-amplification (FIGS. 27A and 27B). This could be due to the rough foliage topography which makes consistent swabbing challenging. Thus, method I was excluded and methods 2 and 3 were used for establishing background levels of fecal contamination in the fresh produce fields.

To establish background levels of Bacteroidales in fresh produce fields during the growing seasons, samples were collected between May 2021 and August 2021 over two growing seasons in two different romaine lettuce commercial fields in Salinas, California, United States. The fields were labeled with row and column numbers with the distance between each row and column to be 6 meters. Samples were collected at the intersection of each row and column (approximately 100 sampling sites per acre of field). Two types of samples were collected at each sampling site 1) 25 grams of romaine lettuce leaf sample (approximately four leaves) 2) collection flag sample. The sample size for the romaine lettuce leaf sample was determined following FDA BAM for isolating specific pathogens from fresh vegetable samples. FDA (2021), supra.

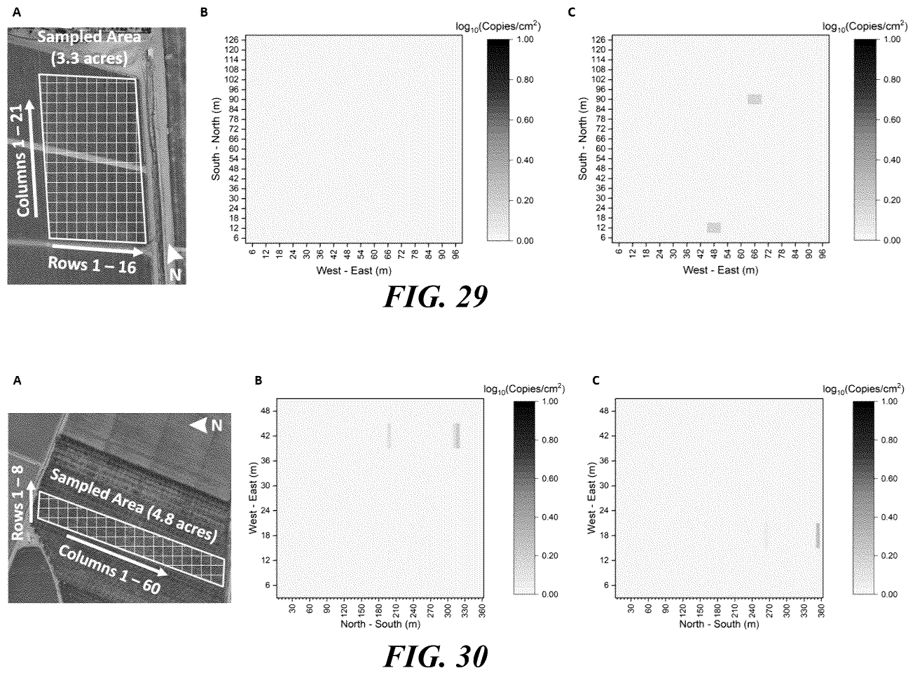

The flags were fabricated and deployed as described above. During the first growing season (May 2021), 336 romaine lettuce leaf samples and 336 collection flag samples were collected over a field size of 3.3 acres (16 rows, 21 columns) (FIG. 29). In the second growing season (August 2021), 480 romaine lettuce leaf samples and 480 collection flag samples were collected over a field size of 4.8 acres (8 rows, 60 columns) (FIG. 30). The samples were collected by individuals wearing Tyvek suits, gloves, and masks to avoid contaminating the samples. Before collecting each sample, 70% ethanol was used to sanitize gloves and sleeves to avoid cross-contamination. Each sample was placed in an individual, pre-labeled Ziploc resealable storage bag (B07NQVYCG3, Amazon, USA). The collected samples were kept on ice and shipped back to West Lafayette, Indiana via FedEx Priority Overnight in a cooler box with ice packs. Following sample collection, the remaining lettuce in the experimental fields was destroyed in the field.

The romaine lettuce leaf samples were processed by a washing and filtering method modified from the FDA BAM for isolating specific pathogens from fresh vegetable samples. FDA (2021), supra. 225 mL of ultrapure water (PURELAB flex, ELGA, USA) was added to the sealed bag with 25 grams of lettuce leaf sample. The bag was hand-shaken for 1 minute to elute any bacteria into the solution. The wash solution was then filtered using a 90 mm, 0.22 μm, cellulose acetate (CA) membrane (FBM090CA022, Filter-Bio, China). The filtered membrane was immersed into 1 mL of nuclease-free water inside a 2 mL centrifuge tube and the tube was vortexed at maximum speed for 1 minute. Finally, the tube was centrifuged at 10,000 rpm for 1 minute to recover the resuspension. The membrane was removed from the tube after centrifugation. Each collection flag was swabbed using a wet polyester-tipped swab (263000, BD BBL, USA) and was resuspended in 200 μL nuclease-free water. All samples were kept at −20° C. until the experiment.

Based on Bacteroidales qPCR assays on a total of 1632 samples (lettuce leaves and collection flags, over a total of two trips), heat map contours of distribution of fecal contamination in the fields were built (FIGS. 29 and 30). In both cases, the levels of Bacteroidales were extremely low as expected for background measurements in clean low-risk fields (i.e., fields away from animal operations in areas normally used for commercial production). This suggests that extremely low background levels of Bacteroidales are present in the soil naturally.

Microbial source tracking was also conducted on samples that returned a concentration higher than 1 copy/reaction (17 samples) for universal Bacteroidales. Since the host-specific populations represent a small group within the general Bacteroidales population, a digital PCR (dPCR) method was adopted for the microbial source tracking experiment to achieve a higher level of sensitivity by amplifying only Bacteroidales derived from a single host species. Bernhard & Field (2000), supra; Lamendella et al., Assessment of fecal pollution sources in small northner-plains watershed using PCR and phylogenetic analyses of Bacteriodetes 16S rRNA gene, FEMS Microbiology Ecology 59 (3): 651-660 (2007); Milbury et al., Determining lower limits of detection of digital PCR assays for cancer-related gene mutations, Biomolecular Detection & Quantification 1 (1): 8-22 (2014). Due to the inherent nature of dPCR, the assay has a high tolerance to biological inhibitors and has better performance on trace detection for a minority target. Milbury et al. (2014), supra; Perkins et al., Chapter Three-Droplet-based digital PCR: application in cancer research, In G.S. Makowski (Ed.) Advances in Clinical Chemistry 79:43-91 (2017).

The dPCR reactions were performed in a total volume of 12 μL, containing 3 μL 4× Probe PCR Master Mix (250102, Qiagen, USA) (final concentration 1×), 1.2 μL of 10× primerprobe mix (final concentration 1×, 0.8 μM forward primer, 0.8 μM reverse primer, 0.4 μM FAM probe), 2.8 μL nuclease-free water, and 5 μL of the template or 5 μL of nuclease-free water for no template control (NTC). 10× primer-probe mix was one of the host-specific qPCR primer probe set in Table 10 (cattle-specific Bacteroidales, swine-specific Bacteroidales, human-specific Bacteroidales, and poultry-specific Bacteroidales). The dPCR reactions were performed in an 8.5K 96-well Nanoplate (250021, Qiagen, USA) on a 5-plex QIAcuity One digital PCR instrument (911021, Qiagen, USA). The thermal cycling conditions were implemented using the following program: initial denaturation at 95° C. for 2 minutes, followed by 40 cycles of 95° C. for 15 seconds, 55° C. for 15 seconds, and 60° C. for 30 seconds.

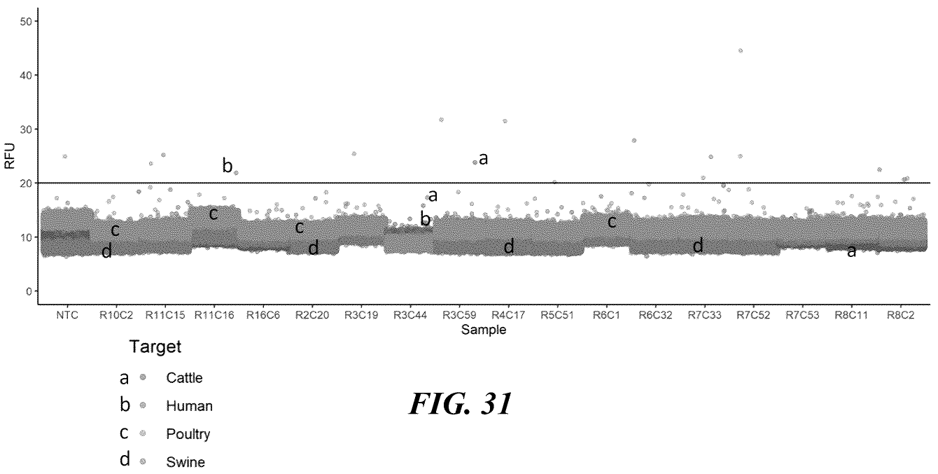

Following plate preparation, the system partitioned each sample into approximately 8500 partitions, with approximately 8300 valid counts. Each partition was individually sealed following 40 cycles of thermocycling. The plate was then imaged to count the number of positive/fluorescent partitions for each sample. The fluorescent threshold was determined to be 20 relative fluorescence units (RFU) based on the NTC. 16 partitions were counted as positive, including 2 positive partitions for cattle-specific Bacteroidales, 3 positive partitions for swine-specific Bacteroidales, 2 positive partitions for human-specific Bacteroidales, and 9 positive partitions for poultry-specific Bacteroidales (FIG. 31). Due to the low copy number of host-specific Bacteroidales, a determination regarding microbial source tracking was not possible.

To establish background levels of Bacteroidales in fresh produce fields during the harvesting seasons, samples were collected in July 2022. 246 swab samples from fresh-pack lettuce harvesters and 210 swab samples from processed lettuce harvesters were collected. The collected samples were shipped back to West Lafayette, Indiana to perform molecular tests.

For processed lettuce harvesters, the conveyer belt, conveyer belt wall, curtain, tunnel, elevator, and funnel surfaces were all swabbed. The sampling sites were selected on the basis of how lettuce comes in contact with the harvester surfaces: from the ground to the collection box.

For fresh pack harvester samples, a 10*10 cm² area of the packing table was swabbed, both before and after harvesting (FIGS. 27A-27B).

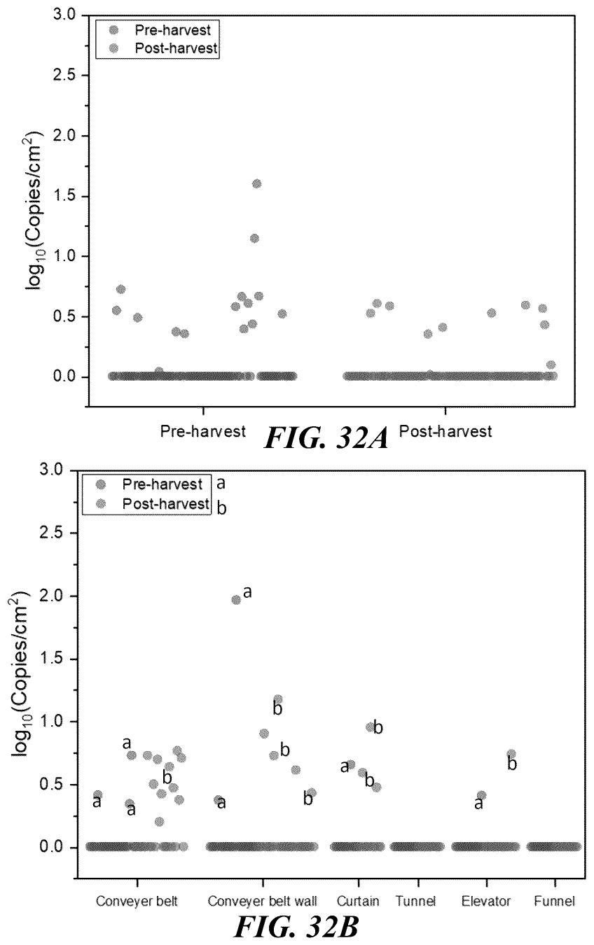

For both types of harvesters, the surfaces were swabbed using a BD CultureSwab™ Sterile Double Swab (220135, BD, US), followed by resuspension in 500 μL nuclease-free water. The team conducted qPCR assays to detect Bacteroidales as markers of fecal contamination on all 456 samples (FIGS. 32A and 32B).

For fresh pack lettuce harvester samples, the qPCR results showed a significant difference in Bacteroidales levels between preharvest (mean=10\hat{ }0.11 copies/cm², SD=10\hat{ }0.28 copies/cm²) and post-harvest (mean=10\hat{ }0.04 copies/cm², SD=10\hat{ }0.14 copies/cm²; p<0.05). The concentration level of Bacteroidales decreased after harvest. For processed lettuce harvester samples, the qPCR results did not show a significant difference in Bacteroidales levels between preharvest (mean=10\hat{ }0.05 copies/cm², SD=10\hat{ }0.23 copies/cm²) and postharvest (mean=10\hat{ }0.12 copies/cm², SD=10\hat{ }0.26 copies/cm²; p=0.06). A trend of decreasing Bacteroidales levels among the harvester sites from the conveyer belt to the Funnel was observed, which corresponded to the travel distance of lettuce from the ground and through the harvester. However, the data was not statistically significant (one-factor analysis of variance (ANOVA), p=0.06)).

### Example 13

### Preliminary Site-Specific Heat Map Evaluations and Weather Station Data

Molecular studies of contamination distribution and concentration integrated with data collected from weather stations were then evaluated, with the goal of providing a risk heat map for fresh produce operations in proximity with animal operations.

Two different weather stations (an Ambient WS-5000 and a Davis Vantage Pro2 Plus) were installed at a swine operation in Indiana (Testbed 1), with data gathered therefrom over a 7-day period and compared. In particular, the following metrics were recorded: temperature, pressure, humidity, daily rain, ultraviolet (UV) and solar radiation, wind speed, and direction, along with air quality data. All data were recorded at 15-minute time intervals and the daily data was averaged. A normality test was run using Sigma Plot 14.0. P-values higher than 0.05 supported there was no significant difference between groups.

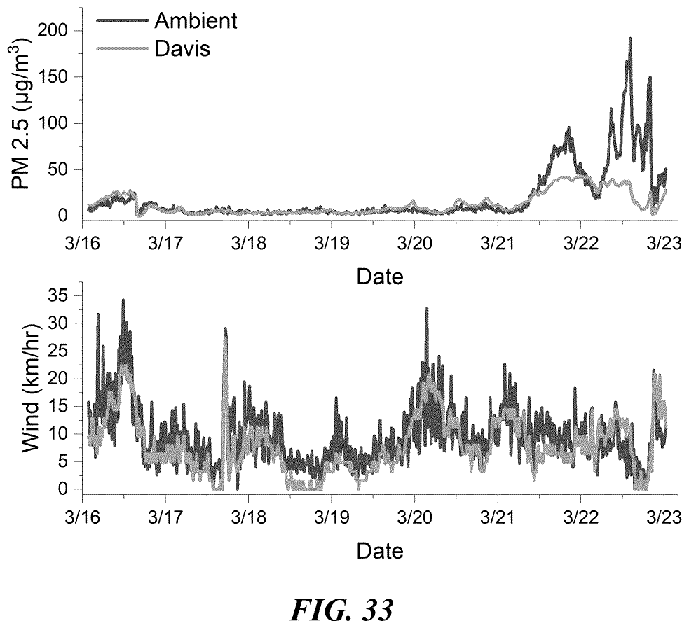

Data comparison of the two weather station models by a paired t-test showed no significant differences (Table 11) and the patterns recorded for a period of 7 days were similar (FIG. 33).

TABLE 11
Statistical difference by t-test in recorded
parameters between two groups.

| Environmental | Normality test |
|---|---|
| parameters | (p-values) |
| Temperature (° C.) | 0.209 |
| Humidity (%) | 0.086 |
| Pressure (Hg) | 0.328 |
| Wind (km/h) | 0.555 |
| High wind (km/h) | 0.819 |
| Solar radiation (W/m²) | 0.096 |
| UV Index | 0.718 |
| Dew point (° C.) | 0.674 |
| PM 2.5 (μg/m³) | 0.753 |

In addition, preliminary heat maps of qPCR data were generated for Bacteroidales at a stone fruit operation in California (Testbed 2) in close proximity to a cattle operation. Briefly, 60 collection flags were placed at 100 meter intervals from east to west and 70 meter intervals from north to south in five blocks, and collected after 7 days. Each flag was swabbed, the swabs resuspended in nuclease-free water and analyzed using a qPCR assay.

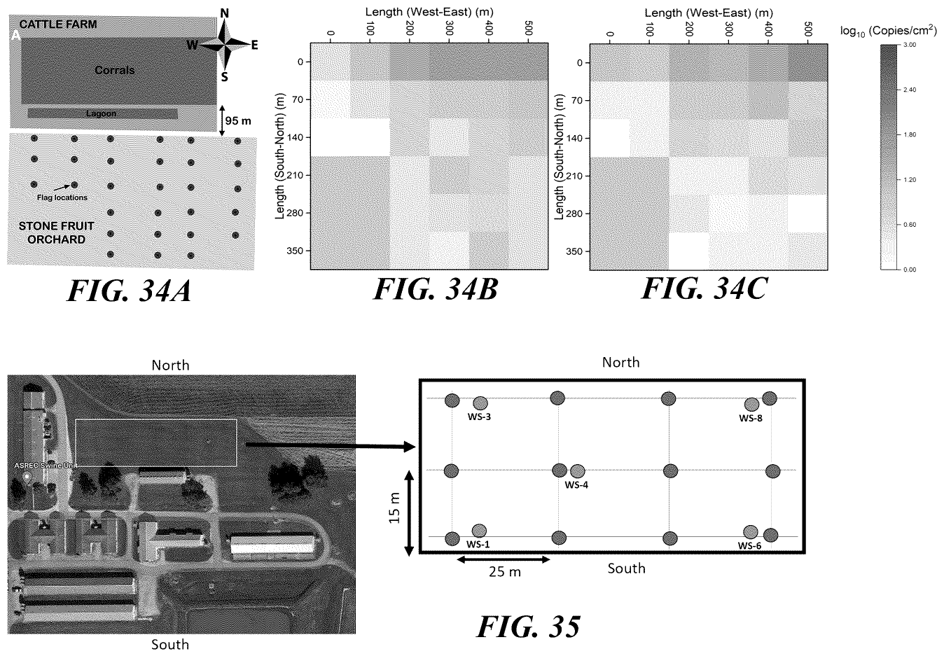

Bacteroidales were detected in all flags and qPCR heat maps were generated depending on the levels of Bacteroidales identified. The heat maps showed higher levels of Bacteroidales (Testbed 2, FIG. 34A) in the northeast direction, possibly due to wind direction (FIGS. 34B and 34C). The first row with collection spots A1, A2, B1, B2, C1, and C2 had higher copy number of Bacteroidales, likely due to their proximity to the animal farm. The results did not demonstrate a significant difference between small and large flags. The levels of Bacteroidales in these stone fruit operations (up to 103 copies/cm²) were higher than what has been seen in the past with background levels for leafy green operations (˜0-2 copies/cm²), supporting that there are differences across industries (e.g., leafy greens vs. stone fruit) such that site-specific risk evaluation would be beneficial.

### Example 14

### Characterization of Testbeds for Distribution of Aerosolized Fecal Contamination

The ability to prepare a risk heat map that incorporates bioaerosol distribution and environmental metadata was then evaluated.

Heat maps of qPCR and wind data were generated for Bacteroidales at a testbed in close proximity to the ASREC swine unit in West Lafayette, Indiana, based on data gathered in a 10-week study. Briefly, five weather stations were placed in the field as shown in FIG. 35, and 120 collection flags were placed at 25-meter intervals from east to west and 15 meter intervals from north to south as shown in FIG. 35. The field of study was 1,000 feet from the swine operation (equating to 3.04.8 meters). Collection flags were also placed in 20 rows, spaced 15 meters apart, extending from the swine unit to the field.

12 flags collected weekly over the 10-weeks. Each flag was swabbed, the swabs resuspended in nuclease-free water and analyzed using a qPCR assay.

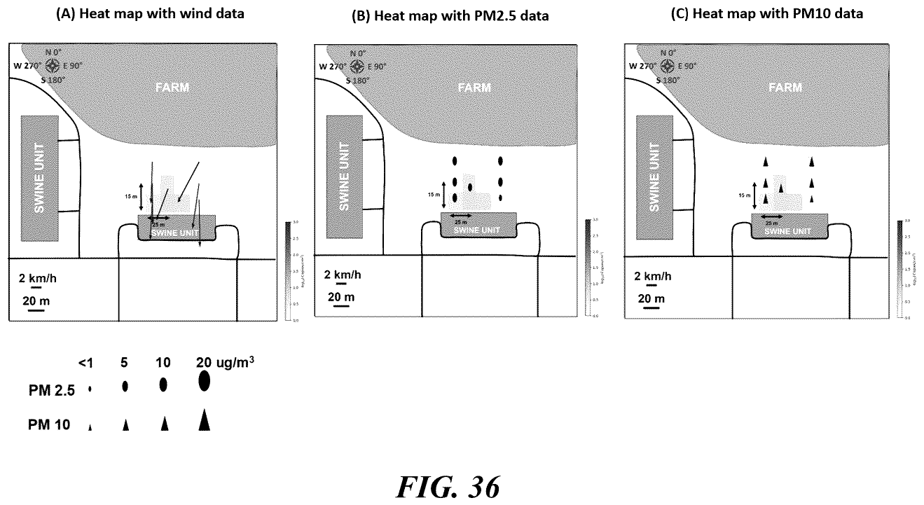

FIG. 36 are heat maps produced from data taken at week 4 of the 10-week study, that incorporates not only the qPCR results, but also wind data (panel A), and particle size data (PM2.5 data, panel B; PM10 data, panel C). The heat map of Bacteroidales concentration shows bioaerosol distribution of fecal matter.

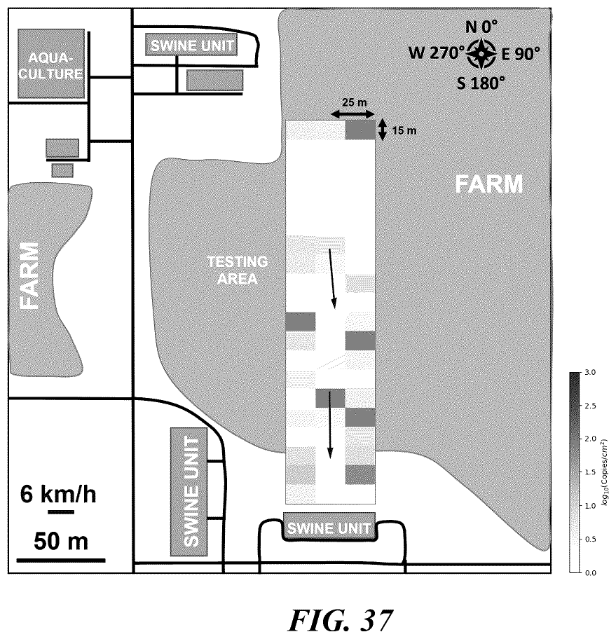

FIG. 37 shows wind data with a heat map of the distance extending between the swine operation and the field. The arrow(s) denote wind data, with the length of arrow and direction of the point denoting wind speed and direction, respectively.

### Example 15

qPCR and Weather Data Integration

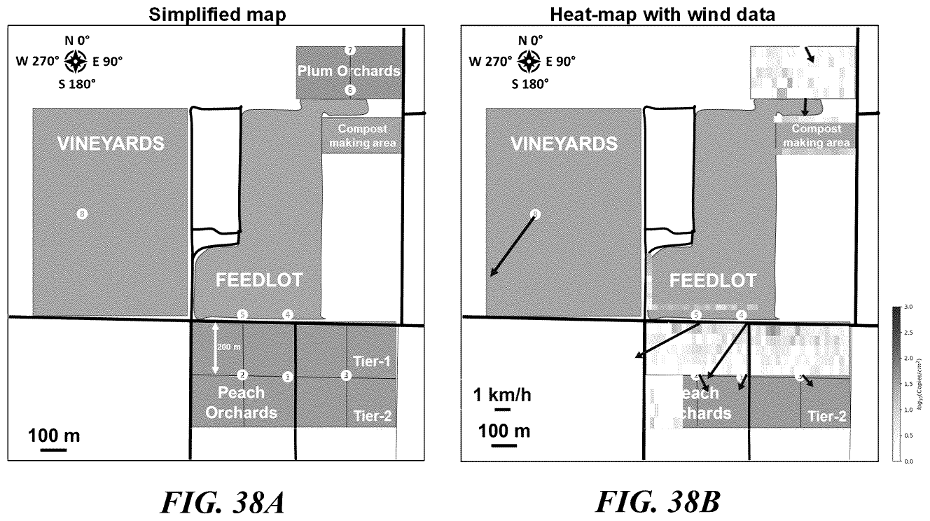

The methods hereof in generating heat maps integrated with weather data were then applied to a vineyard testbed, a plum orchard testbed, and a peach orchard testbed, each in close proximity or adjacent to a feedlot and/or compost area (FIGS. 38A and 38B).

Briefly, 8 weather stations (WS-5000 and AQIN air quality monitors) were installed at each of the locations identified as numbers in a circle in FIG. 38A. The plum orchards had 100 flags/orchards placed 50 meters apart from north (N) to south(S) and 20 meters apart from east (E) to west (W), and the peach orchards had 200 collection flags placed at 50 meters (N to S) and 20 meters (E to W) apart in the area identified as Tier-1. 40 collection flags were placed in the area identified as Tier-2, 30 collection flags were placed in the feedlot, and 20 collection flags were placed within the compost area.

Bioaerosol distribution was determined based on a heat map of Bacteroidales concentrations determined using qPCR. Wind speed and direction were determined based on data collected from the weather stations and averaged over 1 week.

FIG. 38B shows a heat map generated from the collected and integrated data, including wind data (denoted by arrows, with length of arrow and direction of point denoting wind speed and direction, respectively). The heat map of Bacteroidales concentration supports bioaerosol distribution of fecal matter in different directions.

Next, a lettuce field in Arizona was studied using protocols similar to those described above. Bioaerosol distribution was determined based on a heat map of Bacteroidales concentrations determined using qPCR. Wind speed and direction were determined based on data collected from the weather stations and averaged over 1 week. Particle size data was collected and averaged over 1 week using known protocols.

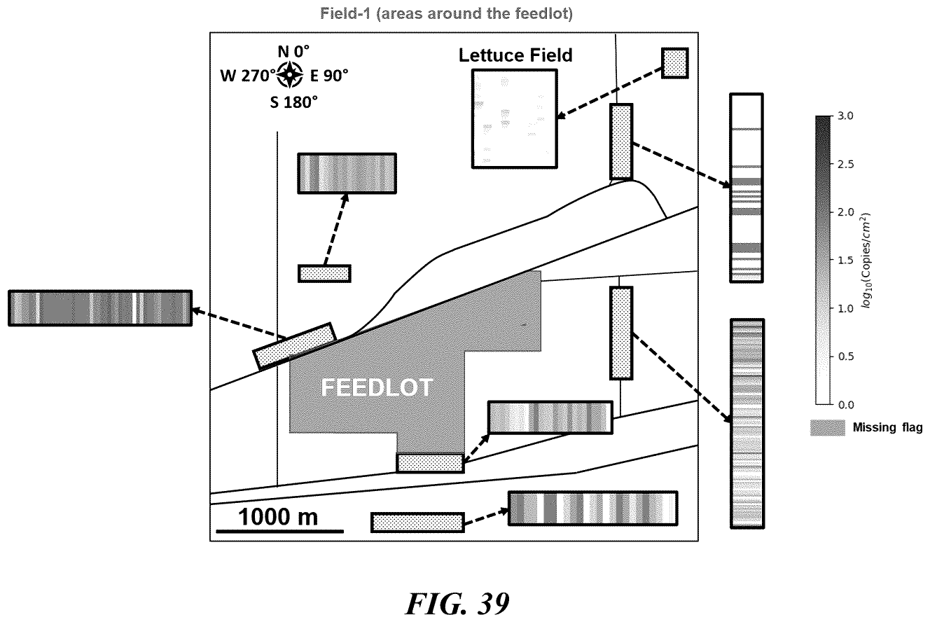

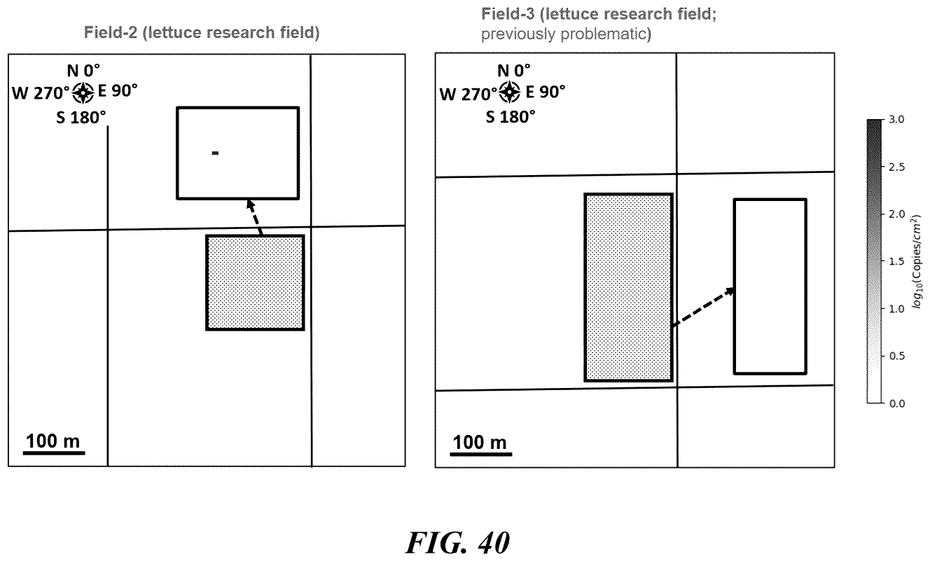

integrating Bacteroidales qPCR data, wind data, and particle size data (PM2.5 and PM10). FIG. 39 shows a simplified map of the location along with heat maps showing log₁₀ (copies/cm²) levels for the samples collected in Field-1. FIG. 40 shows simplified maps of lettuce fields along with heat maps based on the samples collected. In Field-2, Ct value of about 9 could be due to sample contamination. The data for Field-3 suggests there was negligible contamination and, thus, previous issues were unlikely from constant fecal matter.

1. A method for evaluating microbial contamination of an area of land, the method comprising:
obtaining a plurality of samples from various locations in an area of land of interest;
detecting the presence or absence of a genetic target associated with contamination in each sample and, if the genetic target is present, quantifying a concentration of the contamination present in the sample;
generating a heat map of the area of land based on the detection and quantification data;
collecting environmental data from the area of land over a period of time; and
integrating the collected environmental data with the heat map to evaluate site-specific contamination risk in the area of land.

2. The method of claim 1, wherein the contamination comprises a fecal indicator bacteria (FIB).

3. The method of claim 2, wherein the FIB is Bacteroidales, Escherichia coli, or Enterococcus faecalis.

4. The method of claim 1, wherein obtaining a plurality of samples from various locations on an area of land comprises:
positioning a plurality of collection flags for the collection of bioaerosol samples at various locations on the area of land, each collection flag comprising a film affixed to a support at a distance away from an end of the support such that, in use, the support can anchor the film a distance above a surface of an area in which the support is positioned; and
collecting a sample from the film of each of the collection flags.

5. The method of claim 4, wherein the plurality of collection flags comprises 1-200 collection flags per acre of the area of land.

6. The method of claim 1, wherein collecting environmental data comprises collecting weather data from one or more weather stations positioned on the area of land.

7. The method of claim 1, wherein the environmental data comprises temperature data, pressure data, humidity data, daily rain data, ultraviolet (UV) and solar radiation data, wind speed data, wind direction data, air quality data, or any combination of two or more of the foregoing.

8. The method of claim 1, wherein detecting the presence or absence of a genetic target associated with contamination in each sample is performed using a polymerase chain reaction (PCR) assay or an isothermal amplification assay.

9. The method of claim 1, wherein quantifying the presence or absence of a genetic target associated with contamination in each sample further comprises:
providing at least one drop dispensing device comprising:
a liquid holder that defines an interior, the interior in fluid communication with an inlet of the liquid holder and an outlet of the liquid holder, and
a plunger configured to be movable up and down within at least a portion of the interior of the liquid holder such that downward movement of the plunger within the interior causes any fluid contained in the interior to be displaced through the outlet of the liquid holder,
wherein the outlet of the liquid holder further comprises a tip comprising a capillary tube that defines an inner surface in fluid communication with the interior;
providing at least one isothermal amplification assay device comprising two or more paper-based pads positioned in a stacked configuration relative to each other, at least one of the paper-based pads comprising a reaction pad loaded with one or more reagents comprising primer sets for the amplification of a genetic target associated with contamination, and at least one of the paper-based pads comprising a control pad that does not have amplification reagents thereon;
suspending a collected sample in a solvent housed within the liquid holder of the drop dispensing device;
loading the reaction pad of the assay device with the suspended sample by pressing the plunger of the drop dispensing device down to deliver at least a drop of the combined sample and solvent mixture to the reaction pad;
heating the loaded reaction pad of the assay device to initiate amplification of the genetic target if present within the sample; and
detecting a visual result in the heated reaction pad indicative of the presence or absence of the contamination in the sample.

10. The method of claim 9, wherein the primer sets of the assay device are encoded by at least:
SEQ ID NOS: 7-27 and the genetic target is specific to Bacteroidales, and/or
SEQ ID NOS: 1-6 and the genetic target is specific to Escherichia coli.

11. The method of claim 9, wherein the heating step is performed for about 45 to about 120 minutes and the loaded reaction pad is heated to a temperature of at or between 60-70° C.

12. The method of claim 9, further comprising capturing at least one image of the heated reaction pad of the assay device and quantifying the concentration of the contamination in the sample from the at least one image.

13. The method of claim 1, wherein the area of land is in proximity to, or close proximity to, an animal operation.

14. The method of claim 1, wherein the area of land is pre-plant or pre-harvest.

15. The method of claim 1, wherein if contamination is detected, the method further comprises identifying a source of the contamination from the environmental data.

16. The method of claim 1, wherein the heat map comprises a plurality of cells, each of the plurality of cells corresponding to an area in the area of land and associated with a value indicative of a concentration of the contamination in the corresponding area.

17. A method for generating a heat map for a crop-field of interest, the method comprising:
obtaining a plurality of samples from various locations in a field of interest;
detecting the presence or absence of a genetic target associated with contamination in each sample and, if the genetic target is present, quantifying a concentration of the contamination present in the sample;
collecting environmental data from the field of interest over a period of time; and
integrating the collected environmental data and the detection and quantification data to generate a heat map showing contamination in the field of interest.

18. The method of claim 17, wherein contamination density data of different regions in the heat map are presented using color levels.

19. The method of claim 17, wherein the contamination is Bacteroidales.

20. The method of claim 17, wherein if contamination is detected, the method further comprises identifying a source of the contamination from the environmental data.
